(19) United States
(12) Patent Application Publication
(10) Pub. No.: US 2014/0142140 A1
(43) Pub. Date: May 22, 2014

(54) PHARMACEUTICAL COMPOSITION FOR NEUROLOGICAL DISORDERS

(71) Applicant: Philip Bird, Maroochydore (AU)

(72) Inventor: Philip Bird, Maroochydore (AU)

(73) Assignee: GOSFORTH CENTRE (HOLDINGS) PTY LTD, Maroochydore, Queensland (AU)

(21) Appl. No.: 14/131,609

(22) PCT Filed: Jul. 9, 2012
(86) PCT No.: PCT/EP2012/063419

§ 371 Date: Jan. 8, 2014

(30) Foreign Application Priority Data
Jul. 8, 2011 (GB) 1111712.4

(51) Int. Cl. A61K 31/445; A61K 31/4166; A61K 31/20; A61K 45/06

(52) U.S. Cl. CPC A61K 31/445; A61K 45/06; A61K 31/4166; A61K 31/20
USPC 514/319; 514/391; 514/557; 548/321.1

(57) ABSTRACT

An anti-epileptic agent for use in the treatment of a neurological disorder other than epilepsy characterised in that the anti-epileptic agent is the sole active agent and that the daily dose of the anti-epileptic is less than 20% of the minimum daily dose which is effective for mood stabilisation or treatment of epileptic symptoms.

The present invention relates to pharmaceutical compositions for the treatment of neurological disorders, particularly those associated with cognitive processing, such as learning disorders (LD), reading disorders (RD), attention deficit hyperactivity disorder (ADHD), acquired brain injury (ABI), autism, tardive dyskinesia, neurodegenerative disorders (e.g. dementia and Parkinson's disease), spina bifida (SB), chronic pain, post traumatic stress disorder (PTSD), schizophrenia (SCZ) and visual acuity/fatigue.

Cognitive processing enables humans to selectively attend, filter, reflect and prioritise incoming information and integrate this with thoughts and ideas. These processes are particularly important for higher executive function. Executive functions are necessary for the planning and sequencing of goal-directed behaviour. They include the ability to initiate and stop actions, to monitor and change behaviour as needed, and to plan future behaviour when faced with novel tasks and situations. Executive functions include a set of cognitive abilities that control and regulate other abilities and behaviours, to allow humans to anticipate outcomes and adapt to changing situations. Further, the ability to form new concepts and think abstractly is often considered a component of executive function. In particular, this includes the cognitive functions of sequencing, organising and integrating social information and appears to be used during the complex interpersonal interaction which forms the basis of human social communication and interaction. Defective or abnormal cognitive processing can therefore become apparent in behaviours that are controlled by higher executive functioning. Defects in cognitive processing may result in hyper-focusing on a specific topic during conversation and/or an inability to process simultaneously the multiple lines of thought that usually and automatically take place in normal social interaction. Instead the individual may select a preferred, more comfortable, and probably more familiar topic. As a consequence, resistance to or difficulty in following the natural flow of conversation is apparent.

Learning disorders are categorised by difficulties in learning in a typical manner. Such difficulties are thought to arise from an inability of the brain to receive and process information in what is considered to be a normal way.

Developing an ability to read fluently and comprehend standard texts is an acquired process familiar to individuals who have access to the teaching or learning of literacy skills. A diagnosis of a reading disorder is usually made when a patient has an impaired ability when it comes to reading, typically resulting from neurological factors. For reasons related to neurobiological factors, approximately 5-17% of children in the U.S. (Shaywitz & Shaywitz, 2005; Duff & Clarke, 2011) develop a specific learning disability associated with reading and this condition is referred to as developmental dyslexia. Reading disorders include developmental dyslexia, alexia (acquired dyslexia) and hyperlexia. The latter term referring to individuals, who while displaying cognitive and linguistic deficits function with an advanced ability at word recognition skills (Nation, Clarke, Wright & Williams, 2006).

The difficulties presented with dyslexia are usually characterized by deficits in the phonological components of language thus making the recognition of written words and spelling and decoding ability quite difficult (Shaywitz & Shaywitz ibid; Benitez-Burraco, A. (2010).

When Autism Spectrum Disorder (ASD) co-occurs with a reading disorder or dyslexia, considerable variation in reading ability can be expected and this relates to the heterogeneous natures of ASD and the complex genetic and environmental base of dyslexia (Benitez-Burraco, 2010).

Whatever the association is between specific language impairment and autism it is likely that the more effortful either process the more vulnerable they are to impairment. It would seem appropriate to hypothesise that both processes rely heavily on efficient automatic cognitive functioning. According to Solomon et al (2009), cognitive control and the ability to process task-relevant behaviour over competing information is necessary to guide thoughts and actions that are reflective of internal goals.

Attention Deficit Hyperactivity Disorder (ADHD) is the most commonly diagnosed neuro-behavioural disorder of childhood (Willcutt & Pennington, 2000). It is currently defined in the Diagnostic and Statistical Manual of Mental Disorders—Fourth Edition (DSM-IV) (2004) as a disorder of executive functioning, characterised by short attention span, impulsivity and excessive activity. In up to 65% of individuals diagnosed with ADHD there is persistence of some symptoms into adulthood which are associated with significant clinical impairments (Faraone et al., 2006). Results from the National Comorbidity Survey Replication estimate the prevalence of adult ADHD in the United States to be 4.4% (Ronald C Kessler et al., 2006).

Many researchers have reported that the symptoms of inattention, hyperactivity and impulsivity are less pronounced in adults (Biederman, Faraone, Monuteaux, & Biederman, J., Faraone, S. V., & Monuteaux, 2002), and that the phenotype in adults is different to that seen in children (Adler & Cohen, 2004). Further to these behavioural symptoms, adults often complain of impairment across a broader spectrum of daily functions including; difficulty in sustaining attention for reading, poor motivation, over-reacting to frustration, easily bored, procrastination, and an inability to work independently (Murphy & Barkley, 1996). Such symptoms have been reported to impact significantly on academic and work performance and social functioning (Davids, K is, Specka, & Gastpar, 2006).

The inventor has noted that many adults diagnosed with ADHD and receiving adequate, traditional stimulant therapy still remained cognitively and socially impaired. This may also be related to the increasing executive function demands of adulthood including; having to drive, manage money, take care of others, juggle work and home demands and self-regulate nutrition, exercise, and sleep. Adults plan and prioritize constantly on both a micro- and macro level (Weiss et al., 2008).

Unexpectedly and serendipitously improvements were noted in individuals with symptoms of ADHD on ultra low doses of phenyloin which would have not sought to be therapeutically effective. Not only were the higher doses ineffective, they were also associated with significant cognitive side effects. It was only on a dose reduction that the clinical improvements were sustained.

Acquired brain injury (ABI) often results in significant cognitive defects and/or personality changes. In particular, patients may become socially withdrawn and/or their communication deteriorates. They may also develop repetitive motor tics and vocalisations, such as repetitive chanting.

Acquired brain injury is brain damage caused by events after birth. ABI can result in cognitive, physical, emotional, or behavioural impairments that lead to permanent or temporary changes in functioning. These impairments result from either traumatic brain injury (e.g. physical trauma due to accidents, assaults, neurosurgery, head injury etc.) or nontraumatic injury derived from either an internal or external source (e.g. stroke, brain tumours, infection, poisoning, hypoxia, ischemia, encephalopathy or substance abuse). ABI does not include damage to the brain resulting from neurodegenerative disorders.

While research has demonstrated that thinking and behaviour may be altered in virtually all forms of ABI, brain injury is itself a very complex phenomenon having dramatically varied effects where no two persons can expect the same outcome or resulting difficulties. The brain controls every part of human life: physical, intellectual, behavioural, social and emotional. Thus when the brain is damaged, it is likely that some part of a person's life will be adversely affected.

Consequences of ABI often require a major life adjustment around the person's new circumstances, and making that adjustment is a critical factor in recovery and rehabilitation. While the outcome of a given injury depends largely upon the nature and severity of the injury itself, appropriate treatment plays a vital role in determining the level of recovery.

Traumatic brain injury (TBI) is defined as damage to the brain resulting from external mechanical force, such as rapid acceleration or deceleration, impact, blast waves, or penetration by a projectile (Maas, Stocchetti, & Bullock, 2008). Brain function is temporarily or permanently impaired and structural damage may or may not be detectable with current technology.

TBI is one of two subsets of acquired brain injury: the first is brain damage that occurs after birth; the second is non-traumatic brain injury, which does not involve external mechanical force and examples of this include stroke and infection. All traumatic brain injuries are head injuries, but the latter term may also refer to injury to other parts of the head (Blissitt, 2006a) However, the terms head injury and brain injury are often used interchangeably. (The Practice of Forensic Neuropsychology: Meeting Challenges in the Courtroom (Critical Issues in Neuropsychology), 1996). Similarly, brain injuries fall under the classification of central nervous system injuries (Povlishock, 2008) and neurotrauma (Neurotrauma: New Insights Into Pathology and Treatment (Google eBook), 2007). In neuropsychology research literature, in general the term “traumatic brain injury” is used to refer to non-penetrating traumatic brain injuries.

TBI is usually classified based on severity, anatomical features of the injury, and the mechanism (the causative forces) (Povlishock, 2008). Mechanism-related classification divides TBI into closed and penetrating head injury (Maas et al., 2008). A closed (also called nonpenetrating, or blunt) (Blissitt, 2006b) injury occurs when the brain is not exposed (Noggle, 2011). A penetrating, or open, head injury occurs when an object pierces the skull and breaches the dura mater, the outermost membrane surrounding the brain (Noggle, 2011).

Systems also exist to classify TBI by its pathological features (Povlishock, 2008). Lesions can be extra-axial, (occurring within the skull but outside of the brain) or intra-axial (occurring within the brain tissue). Damage from TBI can be focal or diffuse, confined to specific areas or distributed in a more general manner, respectively (Smith, Meaney, & Shull, 1989). However, it is common for both types of injury to exist in a given case (Smith et al., 1989).

Diffuse injury manifests with little apparent damage in neuroimaging studies, but lesions can be seen with microscopy techniques post-mortem (D. H. Smith et al., 1989) (Granacher, 2007), and in the early 2000s, researchers discovered that diffusion tensor imaging (DTI), a way of processing MRI images that shows white matter tracts, was an effective tool for displaying the extent of diffuse axonal injury (Kraus et al., 2007) (Kumar et al., 2009). Types of injuries considered diffuse include oedema (swelling) and diffuse axonal injury, which is widespread damage to axons including white matter tracts and projections to the cortex (Nahum & Melvin, 2001) (McCrea, 2007). Types of injuries considered diffuse include concussion and diffuse axonal injury, widespread damage to axons in areas including white matter and the cerebral hemispheres (Nahum & Melvin, 2001).

Focal injuries often produce symptoms related to the functions of the damaged area (Povlishock, 2008) (Povlishock, 2008). Research shows that the most common areas to have focal lesions in non-penetrating traumatic brain injury are the orbitofrontal cortex (the lower surface of the frontal lobes) and the anterior temporal lobes, areas that are involved in social behaviour, emotion regulation, olfaction, and decision-making, hence the common social/emotional and judgment deficits following moderate-severe TBI (Mattson & Levin, 1990a) (Bayly et al., 2005) (Cummings, 1993) (McDonald, Flanagan, Rollins, & Kinch, 2003). Symptoms such as hemiparesis or aphasia can also occur when less commonly affected areas such as motor or language areas are, respectively, damaged (Basso & Scarpa, 1990) (Mohr et al., 1980).

One type of focal injury, cerebral laceration, occurs when the tissue is cut or torn (Hardman & Manoukian, 2002). Such tearing is common in the orbitofrontal cortex in particular, because of bony protrusions on the interior skull ridge above the eyes (Mattson & Levin, 1990b). In a similar injury, cerebral contusion (bruising of brain tissue), blood is mixed among tissue.

Symptoms are dependent on the type of TBI (diffuse or focal) and the part of the brain that is affected. Unconsciousness tends to last longer for people with injuries on the left side of the brain than for those with injuries on the right (Noggle, 2011). Symptoms are also dependent on the injury's severity. With mild TBI, the patient may remain conscious or may lose consciousness for a few seconds or minutes. Other symptoms of mild TBI include headache, vomiting, nausea, lack of motor coordination, dizziness, difficulty balancing, lightheadedness, blurred vision or tired eyes, ringing in the ears, bad taste in the mouth, fatigue or lethargy, and changes in sleep patterns (NINDA Traumatic Brain Injury Information Page, 2008). Cognitive and emotional symptoms include behavioural or mood changes, confusion, and trouble with memory, concentration, attention, or thinking.

A person with a moderate or severe TBI may have a headache that does not go away, repeated vomiting or nausea, convulsions, an inability to awaken, dilation of one or both pupils, slurred speech, aphasia (word-finding difficulties), dysarthria (muscle weakness that causes disordered speech), weakness or numbness in the limbs, loss of coordination, confusion, restlessness, or agitation (NINDA Traumatic Brain Injury Information Page, 2008). Common long-term symptoms of moderate to severe TBI are changes in appropriate social behaviour, deficits in social judgment, and cognitive changes, especially problems with sustained attention, processing speed, and executive functioning (McDonald et al., 2003) (Textbook of Traumatic Brain Injury, 2004) (Kim, 2002) (Busch, McBride, Curtiss, & Vanderploeg, 2005) (Ponsford, Draper, & Schonberger, 2008). Alexithymia, a deficiency in identifying, understanding, processing, and describing emotions occurs in 60.9% of individuals with TBI (Williams & Wood, 2010). Cognitive and social deficits have long-term consequences for the daily lives of people with moderate to severe TBI, but can be improved with appropriate rehabilitation (Ponsford et al., 2008).

The type, direction, intensity, and duration of forces all contribute to the characteristics and severity of TBI (Maas et al., 2008). Forces that may contribute to TBI include angular, rotational, shear, and translational forces (Hardman & Manoukian, 2002).

Even in the absence of an impact, significant acceleration or deceleration of the head can cause TBI; however in most cases a combination of impact and acceleration is probably to blame (Hardman & Manoukian, 2002). Forces involving the head striking or being struck by something, termed contact orimpact loading, are the cause of most focal injuries, and movement of the brain within the skull, termed noncontact or inertial loading, usually causes diffuse injuries (Povlishock, 2008). The violent shaking of an infant that causes shaken baby syndrome commonly manifests as diffuse injury (Committee on Child Abuse and Neglect, 2001). In impact loading, the force sends shock waves through the skull and brain, resulting in tissue damage (Hardman & Manoukian, 2002). Shock waves caused by penetrating injuries can also destroy tissue along the path of a projectile, compounding the damage caused by the missile itself.

Damage may occur directly under the site of impact, or it may occur on the side opposite the impact (coup and contrecoup injury, respectively) When a moving object impacts the stationary head, coup injuries are typical (Morrison, King, Korell, Smialek, & Troncoso, 1998), while contrecoup injuries are usually produced when the moving head strikes a stationary object (Poirier, 2003).

The trauma occurring from these injuries can occur when the head is accelerated and decelerated abruptly in space, particularly when accompanied by a torsional head movement, and when strain forces are applied to nerve fibers (axons) throughout the brain (Lewis, Volk, & Hashimoto, 2004) (Costa & Guidotti, 1996). The resulting axonal strain injuries are collectively referred to as diffuse axonal injury (DAI), and can contribute to the severity of injury. Axons that project up from the brain stem are particularly vulnerable, and although referred to as diffuse axonal injury, the supra-tentorial injury usually include functional disturbances marked by difficulties with cognitive processing speed, multitasking, and cognitive endurance (Lux, 2007).

The cognitive and behavioural manifestations of TBI are thought to include a number of pathologies including altered neuronal homeostasis due to disruption of the blood-brain barrier (BBB), excessive release of excitatory neurotransmitters, axonal and dendritic disruptions, neuroinflammation, posttraumatic seizures (PTS), and cell death (Bramlett & Dietrich, 2007; Faden, Demediuk, Panter, & Vink, 1989; Kadhim, Duchateau, & Sébire, 2008; Ommaya & Gennarelli, 1974). The somatosensory cortex (neocortex) seems to be particularly susceptible to the development of unrestrained excitation.

Normal activity in the central nervous system is regulated by the critical balance between synaptic excitation and inhibition. The latter cortical inhibitory circuits are comprised of interneurons that release GABA onto pyramidal neurons (the principal cells of the neocortex) and cause a membrane hyperpolarisation that counterbalances excitatory inputs.

One factor contributing to this vulnerability may be an intrinsic limit on the recruitment of GABA inhibition, such that rising excitation can build to levels that exceed the capacity of inhibitory mechanisms to contain. Thus, inhibitory interneurons may play a key role in maintaining the stability of cortical network activity. Well-known modulators of inhibition include agents, which prolong or enhance the actions of GABA on pyramidal cells; these have found applications as effective anticonvulsants, mood stabilisers, sedatives and tranquilizers, and examples of which are Valproate, Phenobarbital and Clonazepam. (Vicini et al., 1986) (Hashimoto et al., 2003)

However, GABA-enhancing drugs used at usual therapeutic doses also produce cognitive impairments, such as amnesia, that presumably result from amplified GABAergic inhibition impeding normal cortical function (Costa and Guidotti, 1996).

Serendipitously we have observed that the GABA enhancing drugs Valproate and Phenyloin used at ultra low doses appear to act as potential nootropic (cognition-enhancing) drugs. These antiepileptic drugs have also been investigated as potential neuroprotective agents. If in addition to this they can successfully modulate these damaged GABA interneurons this may provide a safe therapeutic treatment providing both an improvement in cognitive function and an additional neuroprotective intervention.

It is therefore possible that the Ultra low dose Phenyloin and Valproate may act as nootropic agents through their actions on inhibitory interneurons and thus, they might serve as a new pharmacological approach to boost inhibitory cell output and counter states of hyperexcitation. The therapeutic action of these inhibitory agents appears to be dose dependent, higher doses associated with impaired neuroplasticity; this factor has previously limited their potential as nootropic agents. (Bales, Wagner, & Kline, 2009) Often early treatment in the course of the recovery can do little to disentangle the drug effects from the natural recovery in this period (Whyte, 2010)

Autism is a disorder or neural development characterised by impaired social interaction and communication, and by restricted and repetitive behaviour.

The preceding two decades has witnessed a rapid increase in the worldwide prevalence of Autism or Autistic Disorder (AD). Autism is derived from the Greek word “auto” meaning “self” and this can be ascribed to the active detachment behaviour noted in many individuals with Autism (Lombardo & Baron-Cohen, 2011). Autism is a severe and complex neurodevelopmental disorder characterized by a triad of core symptoms including impairments in communication, ability to interact with others and a preoccupation with routines or repetitive behaviours (Matson et al., 2011; (Gomot & Wicker, 2012). The effects of Autism commence within the first three postnatal years, are pervasive and remain with some degrees of remission throughout life.

Autism is assigned to a spectrum of disorders that are referred to as Autism Spectrum Disorders (ASD) and are distinguishable in the severity of symptoms (Erdmann, 2011). Autism represents the most severe manifestations of this group that includes Asperger Syndrome and Pervasive developmental disorder not otherwise specified (PDD-NOS) and ASD is diagnosed on the basis of behavioural symptoms (Betancur, 2011; (Tager-Flusberg, 2010)). Typically identified in the preschool years, these diagnostic symptoms include; unusual eye contact, limitations in facial expression directed to other people, atypical social engagement and responsiveness, difficulty with peer relationships, lack of awareness of other peoples thoughts and feelings, poor communication skills, difficulty initiating social contacts through verbal or non-verbal means, rigid or unusual behaviours and restricted interests (Tager-Flusberg, 2010).

### Primacy of Communication

It is well established in the ASD literature of the limiting effects that ensue from a non-normative developing repertoire of language and communication function. In a confirmed diagnosis of ASD, the individual may have to contend with numerous language related deficits that relate to expressive language, receptive vocabulary, comprehension of extensive directions, the initiation of communication and engaging in reciprocal conversations (Forde, Holloway, Healy & Brosnan, 2011).

Furthermore this can have a profound effect on the life trajectory of a person in terms of their ability to socially interact with others and their overall quality of life. While symptom severity and comorbidity will be a determining factor in the level of language and speech attainment, functional communication skills fail to develop for many younger individuals with ASD or a related disorder and this in turn can progress into adulthood (Forde et al). However adaptive strategies for these adults may have developed in an attempt to lessen the degree of the communication deficit.

### Autism and Aetiology

Autism is a condition of multiple aetiologies distributed across genetic, neuroanatomical, and behavioural domains that ultimately results in alterations in brain connectivity (Müller, 2007). The cerebellum is an ideal structure to investigate connectivity due to its simple cytoarchitecture, highly ordered topographic circuitry and its multifocal intrinsic GABAergic neurotransmission.

Multiple lines of evidence, including genetic and imaging studies, suggest that the anterior cingulate cortex (ACC) and GABA system may be affected in autism. The benzodiazepine binding site on the GABA_{A} receptor complex is an important target for pharmacotherapy and has important clinical implications. These findings suggest that in the autistic group this down regulation of both benzodiazepine sites and GABA_{A} receptors in the ACC may be the result of increased GABA innervations and/or release disturbing the delicate excitation/inhibition balance of principal neurons as well as their output to key limbic cortical targets. Such disturbances likely underlie the core alterations in socio-emotional behaviours in autism (Oblak A, Gibbs T. T, & Blatt G. J., 2009).

A model for autism has been posited by Rubenstein & Merzenich (2003) that suggests an increasing in the ratio of excitation/inhibition in key neural systems, either genetically or epigenetically and is a common pathway for causing autism. An imbalance of excitation and inhibition could be due to increased glutamatergic (excitatory) signalling, or to a reduction in inhibition due to a reduction in GABAergic signalling.

Hussman (2001) has earlier suggested that suppressed GABAergic inhibition is a common feature of the autistic brain. Such a problem could also be exacerbated by abnormal modulatory control of the learning and memory processes that enable and regulate the normal progressive differentiation and elaboration of information processing in the developing brain, because progressive functional differentiation increases processing reliability and representational salience, and thereby reduces process noise (Zhang, Bao, & Merzenich, 2001) (Tallal, Merzenich, Miller, & Jenkins, 1998), (Rubenstein & Merzenich, 2003)

### Possible Evolutionary Perspective

A perspective on the evolution of autism is provided by the Polyvagal theory (Porges, 2003a). Polyvagal theory postulates that through three stages of phylogeny, mammals, especially primates, including humans, have evolved a functional neural organization that regulates emotions and social behaviour. The vagus, i.e., the 10th cranial nerve is a major component of the autonomic nervous system that plays an important role in regulating emotions and social behaviour.

The Polyvagal Theory emphasizes that physiological states support different classes of behaviour. For example, a physiological state, characterized by a vagal withdrawal, would support the mobilization behaviours of fight and flight. In contrast, a physiological state, characterized by increased vagal influence (via pathways originating in the nucleus ambiguous) on the heart, would support spontaneous social engagement behaviours. Further, the theory emphasizes the functional and structural links between neural control of the striated muscles of the face and the smooth muscles of the viscera (Porges, 2007). This would provide an explanation for the autonomic and involuntary control of the facial muscles involved in non-verbal communication.

The most phylo genetically primitive component, the immobilization system, is dependent on the unmyelinated vagus, which is shared with most vertebrates. With increased neural complexity resulting from phylogenetic development, the organism's behavioural and affective repertoire is enriched. The three circuits can be conceptualized as dynamic, providing adaptive responses to safe, dangerous, and life-threatening events and contexts.

The human nervous system has evolved in line with other mammals to enable survival in dangerous and life threatening contexts. To accomplish this adaptive flexibility, the human nervous system retained two more primitive neural circuits to regulate defensive strategies (i.e., fight/flight and freeze behaviours) (Porges, 2007).

It is important to note that social behaviour, social communication, and visceral homeostasis are incompatible with the neurophysiological states and behaviours promoted by the two neural circuits that support defence strategies. Thus, via evolution the human nervous system retains three neural circuits, which are in a phylogenetically organized hierarchy. Porges 2007, suggests that these three circuits are organized and respond to challenges in a phylogenetically-determined hierarchy consistent with the Jacksonian principle of dissolution (Jackson, 1873).

Jackson proposed that in the brain, higher (i.e., phylogenetically newer) neural circuits inhibited lower (i.e., phylogenetically older) neural circuits and “when the higher are suddenly rendered functionless, the lower rise in activity” (Taylor, 1958). In this hierarchy of adaptive responses, the newest circuit is used first, and if that circuit fails to provide safety the older circuits are recruited sequentially. Porges has proposed (Porges, 1998) (Porges, 2001) (Porges, 2003b) the neural pathways originating in several cranial nerves that regulate the striated muscles of the face and head (i.e., special visceral efferent) and the myelinated vagal fibers from the neural substrate of the Social Engagement System.

The social communication system (i.e., Social Engagement System, see below) is dependent upon the functions of the myelinated vagus, which serves to foster calm behavioural states by inhibiting the sympathetic influences to the heart and dampening the HPA axis (Porges, 2009). The Social Engagement System controls the cortical upper motor neurons that regulates brainstem nuclei (lower motor neurons) to control eyelid opening (e.g., looking), facial muscles (e.g., emotional expression), middle ear muscles (e.g., extracting human voice from background noise), muscles of mastication (e.g., ingestion), laryngeal and pharyngeal muscles (e.g., prosody and intonation), and head turning muscles (e.g., social gesture and orientation). Collectively, these muscles function both to enable social engagement and to filter and thus enhance the information processed (Porges, 2007).

The neural pathways that raise the eyelids also tense the stapedius muscle in the middle ear, which facilitates hearing human voice (Borg & Counter, 1989). Thus, the neural mechanisms for making eye contact are shared with those needed to listen to human voice. As a cluster, difficulties in gaze, extraction of human voice, facial expression, head gesture and prosody are common features of individuals with autism and other psychiatric disorders (Porges, 2007).

We have treated many individuals with both autistic spectrum disorders and social communication disorders. A primary impairment associated with these disorders is the inability to communicate, which can lead to frustration and behavioural disturbance. Many children are diagnosed in childhood with these conditions and experience significant handicap and impairment both socially and educationally. Because of the frequent behavioural disturbance they are commenced on psychotropic medications. Two medications which have been approved by the food and drug administration in the United States of America as well as other countries for the treatment of behavioural disturbance in autism are Risperidone and Aripiprazole. Although effective treatments, they are associated with side effects including sedation, cognitive impairment, weight gain and metabolic disturbance. Many individuals have experienced these side effects with only moderate improvement in behaviour with little or no improvement in their communication. Many also have a comorbid diagnosis of ADHD and have been treated with stimulants and non-stimulants. This has resulted in improvements in attention, concentration and impulsivity, although again little benefit in their abilities to communicate.

On the addition of Ultra low dose phenyloin we have noted a frequent instant improvement in both their non-verbal and verbal communication. These benefits occurred at Ultra low doses of phenyloin and in the case of autistic spectrum disorders are frequently below 5 mg.

These have included;
Improved eye contact both when listening and speaking
Enhanced prosody and complexity of speech
Improved organisation and sequencing of conversation
Enhanced timing and appropriateness of non-verbal communication in contrast to the frequent delayed and exaggerated responses characteristic of these conditions, examples of these have included excessive head nodding, smiling, facial grimacing, use of hands and arms. These are placed by subtle but more immediate and responsive movements of the small muscles of the face particularly around the eyes and forehead. These are involuntary and reflexive, providing more empathic and intuitive interaction.
Subsequent descriptions of improvements in ability to listen and respond verbally with a reduction in the internal dialogue, this previously had been disabling resulting in extreme difficulty in communicating thoughts and ideas.

Post-traumatic stress disorder (PTSD) affects 8% of Americans at some time in their lives and is associated with considerable morbidity (R C Kessler, Sonnega, Bromet, Hughes, & Nelson, 1995). Developing effective treatments for PTSD is of critical importance. Large placebo-controlled trials revealed efficacy for the selective serotonin reuptake inhibitors (SSRI), sertraline (Brady et al., 2000) and paroxetine (Tucker et al., 2001) in PTSD, but not all patients respond optimally to SSRI treatment.

Although in a recent open study Phenyloin treatment resulted in a significant 6% increase in right brain volume (p<0.05). Increased hippocampal volume was correlated with reductions in symptom severity as measured by the Clinician Administered PTSD Scale and improvements in executive function as measured by the Trails Making test. However, treatment associated with improvements in memory and cognition did not achieve statistical significance. These findings suggest that phenyloin treatment may be associated with changes in brain structure in patients with PTSD. Treatment was begun at 300 mg per day divided into three doses and increased to 400 mg/day if plasma levels were sub-therapeutic. (Bremner et al., 2005).

We have noted that patients with post traumatic stress disorder have difficulty controlling the intrusive and distressing memories. This frequently results in poor concentration, irritability and frustration. It would appear that they have a reduced ability to automatically/effortlessly control their re-experiencing phenomena relying on effortful control. This leads to cognitive fatigue and being overwhelmed by their memories. This pattern of functioning is not dissimilar to the failure to habituate noxious stimuli. Although it has been previously reported that antiepileptic medications are useful for the treatment of post-traumatic stress disorder the doses used were within the therapeutic range. Unexpectedly and contrary to usual clinical practice we have noted that the use of Ultra low dose phenyloin has been associated with significant improvements in functional capacity as well as a reduction in the symptoms of PTSD.

The presence of schizophrenia as a major mental disorder now affects 1% of the world's population and renewed emphasis on cause and treatment modalities have prompted many researchers in the area to realign their former psychogenic models of investigation to a neurobiological structure of inquiry. More specifically, the importance of neurocognitive assessment in determining greater clinician understanding of cognitive domain deficits has occurred thus permitting greater accuracy for pharmacological treatment provision. According to (Reichenberg, 2005) (Arnott, Sali, & Copland, 2011), cognitive deficits remain the key feature of schizophrenia and a primary cause of long-term disability.

Cognitive deficit is a stable, traits like condition, independent of psychotic symptoms and mostly unaffected by antipsychotic treatment. Cognitive deficits are associated with social deficits. In a meta-analysis of 37 studies (M F Green, Kern, Braff, & Mintz, 2000), it was found that cognitive impairment accounted for 20%-60% of the variance in functional outcome for individuals with schizophrenia. Attention, verbal learning and fluency are related to successful performance of social skills (Silverstein, Schenkel, Valone & Nuernberger, 1998).

Another prominent factor which may affect functional outcome in schizophrenia is impaired facial affect recognition. This has been linked to negative symptom severity and poor functional outcome (Brekke, Kay, Lee, & Green, 2005; Heimberg, Gur, Erwin, Shtasel, & Gur, 1992; Heimberg et al., 1992; Silver, Goodman, Knoll, & Isakov, 2004; Manuscript et al., 2006; Morris, Weickert, & Loughland, 2009). There is increasing evidence from treatment studies that emotional face recognition deficits may be remediated (Morris et al., 2009) both by behavioural (Russell, Green, Simpson, & Coltheart, 2008) and pharmacological means (Guastella, Mitchell, & Dadds, 2008) reported that oxytocin increased the gaze of healthy individuals to the eye region of neutral face. Recent evidence suggests that reduced oxytocin levels may be related to negative symptoms, social withdrawal and isolation in schizophrenia (Keri, Kiss, & Kelemen, 2009).

The wide-ranging cognitive deficits observed in individuals with schizophrenia include communication and oral language problems. Evidence of schizophrenia-related reading difficulties has also emerged (e.g. (Manuscript et al., 2006)). It has been advanced by Condray and others Crow et al 1995, (McNab & Klingberg, 2008), that language disorder is increasingly understood as an important characteristic of schizophrenia. Patients with schizophrenia, like those with dyslexia, show deficits in early auditory processing including, for example, deficits in tone matching (Daniel C. Javitt, Shelley, Silipo, & Lieberman, 2000), mismatch negativity generation (D C Javitt, Doneshka, Grochowski, & Ritter, 1995) and the ability to detect phonetic boundaries (Cienfuegos, March, Shelley, & Javitt, 1999).

Reading deficits are predicted strongly by recent research demonstrating impaired functioning of the magnocellular visual pathway in schizophrenia. The magnocellular (M) pathway is one of two primary low-level visual pathways in the human brain, and is primarily responsible for processing low spatial frequency and motion information, and for organising visual space. Magnocellular processing deficits have been extensively linked to dyslexia (Demb, Boynton, Best, & Heeger, 1998); (Talcott J B, Hansen P C, Willis-Owen C, McKinnell I W, Richardson A J, 1998); (Romani et al., 2001) (Romani et al., 2001); (Ridder, Borsting, Cooper, McNeel, & Huang, 1997).

These disturbances of the cognitive processes, such as working memory, are now regarded as core features of schizophrenia, but available pharmacological treatments produce little or no improvement in these cognitive deficits. These cognitive deficits appear to reflect a disturbance in executive control, the processes that facilitate complex information processing and behaviour, and that include context representation and maintenance, functions dependent on the dorsolateral prefrontal cortex (DLPFC). Studies in non-human primates indicate that normal working memory function depends upon appropriate GABA neurotransmission in the DLPFC, and alterations in markers of GABA neurotransmission are well documented in the DLPFC of subjects with schizophrenia (Lewis et al., 2004).

Despite the advances in our understanding of the neurocognitive deficits but as yet, not widely applicable, evidence-based treatments are available to the clinician. The site of action of phenyloin in its antiepileptic activity appears to hinge on the inhibition of voltage-sensitive Na+ channels in the plasma membrane of neurons undergoing seizure activity including the GABA, receptors, which are found on the GABA ergic interneurons (Tunnicliff, 1996).

It is known that sodium valproate and phenyloin appear to exert a negative effect on cognitive functioning particularly when used at high dosage (Mula & Trimble, 2009). Therefore one would expect that these medications used in schizophrenia would further increase cognitive impairment.

Schizophrenia is a mental disorder characterized by a breakdown of thought processes and by poor emotional responsiveness. It most commonly manifests itself as auditory hallucinations, paranoid or bizarre delusions, or disorganized speech and thinking, and it is accompanied by significant social or occupational dysfunction. The onset of symptoms typically occur in young adulthood, with a global lifetime prevalence of about 0.3-0.7% (van Os & Kapur, 2009). Diagnosis is based on observed behaviour and the patient's reported experiences.

Genetics, early environment, neurobiology, and psychological and social processes appear to be important contributory factors; some recreational and prescription drugs appear to cause or worsen symptoms. Current research is focused on the role of neurobiology, although no single isolated organic cause has been found. The many possible combinations of symptoms have triggered debate about whether the diagnosis represents a single disorder or a number of discrete syndromes. Despite the etymology of the term from the Greek roots skhizein “to split” and phren, “mind”, schizophrenia does not imply a “split personality,” or “multiple personality disorder” (which is known these days as dissociative identity disorder)—a condition with which it is often confused in public perception (Picchioni & Murray, 2007). Rather, the term means a “splitting of mental functions”, because of the symptomatic presentation of the illness.

The mainstay of treatment is antipsychotic medication, which primarily suppresses dopamine (and sometimes serotonin) receptor activity. Psychotherapy and vocational and social rehabilitation are also important in treatment. In more serious cases—where there is risk to self and others—involuntary hospitalization may be necessary, although hospital stays are now shorter and less frequent than they once were (Becker & Kilian, 2006).

The disorder is thought mainly to affect cognition, but it also usually contributes to chronic problems with behaviour and emotion. People with schizophrenia are likely to have additional (comorbid) conditions, including major depression and anxiety disorders; the lifetime occurrence of substance abuse is almost 50% (Buckley, Miller, Lehrer, & Castle, 2009). Social problems, such as long-term unemployment, poverty and homelessness, are common. The average life expectancy of people with the disorder is 12 to 15 years less than those without, the result of increased physical health problems and a higher suicide rate (about 5%) (van Os & Kapur, 2009).

Many psychological mechanisms have been implicated in the development and maintenance of schizophrenia. Cognitive biases have been identified in those with the diagnosis or those at risk, especially when under stress or in confusing situations (Bentall, Fernyhough, Morrison, Lewis, & Corcoran, 2007). Some cognitive features may reflect global neurocognitive deficits such as memory loss, while others may be related to particular issues and experiences (Bentall et al., 2007) (Kurtz, 2005).

Despite a demonstrated appearance of blunted affect, recent findings indicate that many individuals diagnosed with schizophrenia are emotionally responsive, particularly to stressful or negative stimuli, and that such sensitivity may cause vulnerability to symptoms or to the disorder (Cohen & Docherty, 2004) (Horan & Blanchard, 2003). Some evidence suggests that the content of delusional beliefs and psychotic experiences can reflect emotional causes of the disorder, and that how a person interprets such experiences can influence symptomatology (B. Smith et al., 2006) (Bell, Halligan, & Ellis, 2006). The use of “safety behaviours” to avoid imagined threats may contribute to the chronicity of delusions (Freeman et al., 2007). Further evidence for the role of psychological mechanisms comes from the effects of psychotherapies on symptoms of schizophrenia (Kuipers et al., 2006).

Schizophrenia is often described in terms of positive and negative (or deficit) symptoms (Professor, 2002). Positive symptoms are those that most individuals do not normally experience but are present in people with schizophrenia. They can include delusions, disordered thoughts and speech, and tactile, auditory, visual, olfactory and gustatory hallucinations, typically regarded as manifestations of psychosis (Kneisl & Trigoboff, 2008). Hallucinations are also typically related to the content of the delusional theme (Association, 2000). Positive symptoms generally respond well to medication (Association, 2000). Negative symptoms are deficits of normal emotional responses or of other thought processes, and respond less well to medication (Kneisl & Trigoboff). They commonly include flat or blunted affect and emotion, poverty of speech (alogia), inability to experience pleasure (anhedonia), lack of desire to form relationships (asociality), and lack of motivation (avolition). Research suggests that negative symptoms contribute more to poor quality of life, functional disability, and the burden on others than do positive symptoms. People with prominent negative symptoms often have a history of poor adjustment before the onset of illness, and response to medication is often limited (T. Smith, Weston, & Lieberman, 2010) Schizophrenia is associated with subtle differences in brain structures, found in 40 to 50% of cases, and in brain chemistry during acute psychotic states. Studies using neuropsychological tests and brain imaging technologies such as fMRI and PET to examine functional differences in brain activity have shown that differences seem to most commonly occur in the frontal lobes, hippocampus and temporal lobes (The Boundaries of Consciousness: Neurobiology And Neuropathology (Google eBook), 2006). Reductions in brain volume, smaller than those found in Alzheimer's disease, have been reported in areas of the frontal cortex and temporal lobes. It is uncertain whether these volumetric changes are progressive or pre-exist prior to the onset of the disease (Konradi & Heckers, 2003). These differences have been linked to the neurocognitive deficits often associated with schizophrenia (Michael F Green, 2006). Because neural circuits are altered, it has alternatively been suggested that schizophrenia should be thought of as a collection of neurodevelopmental disorders (Insel, 2010).

Particular attention has been paid to the function of dopamine in the mesolimbic pathway of the brain. This focus largely resulted from the accidental finding that phenothiazine drugs, which block dopamine function, could reduce psychotic symptoms. It is also supported by the fact that amphetamines, which trigger the release of dopamine, may exacerbate the psychotic symptoms in schizophrenia (Laruelle et al., 1996). The influential dopamine hypothesis of schizophrenia proposed that excessive activation of D2 receptors was the cause of (the positive symptoms of) schizophrenia. Although postulated for about 20 years based on the D₂ blockade effect common to all antipsychotics, it was not until the mid-1990s that PET and SPET imaging studies provided supporting evidence. The dopamine hypothesis is now thought to be simplistic, partly because newer antipsychotic medication (atypical antipsychotic medication) can be just as effective as older medication (typical antipsychotic medication), but also affects serotonin function and may have slightly less of a dopamine blocking effect (Jones & Pilowsky, 2002).

Interest has also focused on the neurotransmitter glutamate and the reduced function of the NMDA glutamate receptor in schizophrenia, largely because of the abnormally low levels of glutamate receptors found in the post-mortem brains of those diagnosed with schizophrenia (Konradi & Heckers, 2003), and the discovery that glutamate-blocking drugs such as phencyclidine and ketamine can mimic the symptoms and cognitive problems associated with the condition (Lahti, Weiler, Tamara Michaelidis, Parwani, & Tamminga, 2001). Reduced glutamate function is linked to poor performance on tests requiring frontal lobe and hippocampal function, and glutamate can affect dopamine function, both of which have been implicated in schizophrenia, have suggested an important mediating (and possibly causal) role of glutamate pathways in the condition (Coyle, Tsai, & Goff, 2003). But positive symptoms fail to respond to glutamatergic medication (Tuominen, Tiihonen, & Wahlbeck, 2005).

Deficits in social cognition form one of the most significant areas of impairment associated with schizophrenia and are often characterised as the negative symptoms of schizophrenia. There is also evidence suggesting that there is an association between childhood autism and the subsequent diagnosis schizophrenia. This evidence together with the biological function of self-awareness and conscious experience, and how its disturbance in pathology may account for major symptoms in self-regulatory disorders like autism, ADHD and schizophrenia. Therefore it would not be an unreasonable hypothesis that the deficits in social cognition are associated with conditions such as schizophrenia may well benefit from mood stabilising medication. However, the finding that of the low dose phenyloin improved social cognition was unexpected at a dose which would not normally be considered effective.

In individuals with bipolar mood disorder there is evidence of stable and lasting cognitive impairment in all phases of the disorder, including the remission phase, particularly in the following domains: sustained attention, memory and executive functions. (Latalova, Prasko, Diveky, & Velartova, 2011) This is not infrequently results in difficulties in social cognition and maintenance of social relationships.

A recent review reported significant relationships between cognitive impairments and functional outcomes were reported in 12 of the 13 studies (Wingo, Harvey, & Baldessarini, 2009), The quality of which are important to maintain support particularly during periods of illness. Therefore treatment which enhances psychosocial functioning may be associated with a significant positive outcome. Antiepileptic Mood stabilisers have been a mainstay of treatment for bipolar mood disorder, the typical therapeutic dose for sodium valproate in the treatment of bipolar disorder is considered between 1000 mg and 2000 mg a day, although other antiepileptics have been found to be helpful the typical dose is usually considered in a similar range to that required for the treatment of epilepsy. We have noted in some patients with bipolar mood disorder a significant improvement in their executive functioning on the addition of a very low dose of phenyloin. Improvement appears to be consistent that observed in other conditions with enhanced organisation, and sequencing of thought together with enhanced social cognition.

Tardive Dyskinesia (TD) is a disorder resulting in involuntary, repetitive body movements that have a slow or belated onset. The movements often have no purpose and can include grimacing, tongue protrusion, lip smacking, puckering and pursing of the lips, and rapid eye blinking. Rapid movements of the extremities may also occur.

Neurodegeneration is the progressive loss of structure or function of neurons, including the death or functional disablement of neurons in the brain and/or central nervous system.

Neurodegenerative diseases, including Parkinson's, dementia, Alzheimer's disease and Huntington's disease, are discussed in more detail below.

Parkinson's disease is characterised by tremor, rigidity, akinesia or bradykinesia, and loss of postural reflexes, associated with reduced dopamine activity in the brain. It may be classified as follows:
primary (idiopathic) parkinsonism, usually referred to as Parkinson's disease (formerly paralysis agitans)
secondary (acquired) parkinsonism, including postencephalitic parkinsonism, drug-induced parkinsonism, and symptoms associated with manganese poisoning
   ‘parkinsonism-plus’ syndromes where parkinsonism is a feature of other degenerative diseases of the CNS, such as progressive supranuclear palsy and multiple system atrophy.

“Arteriosclerotic parkinsonism” has been used to describe parkinsonism associated with cerebrovascular disease, although this may be confusing since vascular brain damage is not a cause of Parkinson's disease.

The term parkinsonism is often used for the idiopathic form, that is, Parkinson's disease. Parkinson's disease and postencephalitic parkinsonism have been attributed primarily to depletion of striatal dopamine in the basal ganglia as a result of the loss of neurones in the substantia nigra. Striatal dopamine deficiency results in loss of the normal functional balance between dopaminergic and cholinergic activity and the aim of treatment is to increase the former and/or decrease the latter.

The cause of Parkinson's disease is not established, although environmental and genetic factors are probably superimposed on a background of neuronal loss related to ageing. MPTP (1-methyl-4-phenyl-1,2,3,6-tetrahydropyridine), a contaminant of an illicitly produced pethidine analogue MPPP (1-methyl-4-phenyl-4-propionoxypiperidine), causes irreversible parkinsonism similar to Parkinson's disease. This effect appears to follow conversion by monoamine oxidase B to the neurotoxic methylphenylpyridinium ion which is selectively concentrated in dopaminergic neurones in the substantia nigra. It has been proposed that free radicals produced during normal metabolism of dopamine in the brain by monoamine oxidase B might be similarly neurotoxic to dopaminergic neurones in the substantia nigra (the oxidant stress hypothesis). This has led to concern that use of levodopa, by increasing the supply of dopamine, might therefore exacerbate neurodegeneration and hasten the progression of Parkinson's disease but compelling evidence of such an effect is lacking.

Drug-induced parkinsonism can arise from depletion of presynaptic dopamine, as with reserpine and tetrabenazine, or from blockade of postsynaptic dopamine receptors in the striatum, as by antipsychotics and some antiemetics such as metoclopramide. It is generally reversible on drug withdrawal or dose reduction and may sometimes disappear gradually despite continuous drug therapy. Although the use of levodopa to overcome antipsychotic-induced blockade of dopamine receptors might appear rational, it has generally been reported to be ineffective or to increase psychiatric symptoms. Antimuscarinics may provide relief from the extrapyramidal symptoms that occur as adverse effects of antipsychotic therapy; however, they do not relieve the symptoms of tardive dyskinesia and should be withdrawn if it develops.

There is no cure for Parkinson's disease. Although the possibility of using drug therapy to slow neurodegeneration is being investigated, no drug so far has a proven neuroprotective effect. Treatment is palliative and symptomatic and consists mainly of drug therapy supplemented when necessary with physical treatment such as physiotherapy and speech therapy. Surgery is occasionally used and there is growing interest in the use of transplantation and in electrical devices for the control of tremor.

The most widely used form of treatment is L-dopa in various forms. L-dopa is transformed into dopamine in the dopaminergic neurons by L-aromatic amino acid decarboxylase (often known by its former name dopa-decarboxylase). However, only 1-5% of L-DOPA enters the dopaminergic neurons. The remaining L-dopa is often metabolised to dopamine elsewhere, causing a wide variety of side effects. Due to feedback inhibition, L-dopa results in a reduction in the endogenous formation of L-dopa, and so eventually becomes counterproductive. The majority of patients respond initially to levodopa and its use has improved the quality and duration of life. However, after 2 years or more, benefit is reduced as the disease progresses and late complications emerge. Apart from dyskinesias and psychiatric effects, a major problem with long-term levodopa treatment is the appearance of fluctuations in mobility, the two predominant forms being ‘end-of-dose’ deterioration (‘wearing-off’ effect) and the ‘on-off’ phenomenon. Thus, views differ as to the best time to start treatment and the dosage to use in order to limit the long-term complications.

Carbidopa and benserazide are dopa decarboxylase inhibitors. They help to prevent the metabolism of L-dopa before it reaches the dopaminergic neurons and are generally given as combination preparations of carbidopa/levodopa (co-careldopa) (e.g. Sinemet, Parcopa) and benserazide/levodopa (co-beneldopa) (e.g. Madopar). There are also controlled release versions of Sinemet and Madopar that spread out the effect of the L-dopa. Duodopa is a combination of levodopa and carbidopa, dispersed as a viscous gel. Using a patient-operated portable pump, the drug is continuously delivered via a tube directly into the upper small intestine, where it is rapidly absorbed. Another drug, Stalevo (carbidopa, levodopa and entacapone), is also available for treatment.

Catechol-O-methyltransferase (COMT) inhibitors, such as entacapone and tolcapone, are selective and reversible inhibitors of COMT, with mainly peripheral actions. They are given as adjunctive therapy to patients experiencing fluctuations in disability related to levodopa and dopa-decarboxylase inhibitor combinations; because of the risk of serious hepatotoxicity, tolcapone should be restricted to when other adjunctive therapy is ineffective or contra-indicated. When levodopa is used with a peripheral dopa-decarboxylase inhibitor, O-methylation becomes the predominant form of metabolism of levodopa; adding a peripheral COMT inhibitor can thus extend the duration and effect of levodopa in the brain, and allow lower and less frequent doses of levodopa. They therefore can help to stabilise patients, especially those experiencing ‘end-of-dose’ deterioration.

Dopamine agonists such as bromocriptine, cabergoline, lisuride, pergolide, pramipexole, and ropinirole act by direct stimulation of remaining postsynaptic dopamine receptors. Dopamine agonists are increasingly used in the early treatment of younger patients with parkinsonism in an attempt to delay therapy with levodopa (younger patients are at an increased risk of motor complications with levodopa). However, their efficacy often decreases after a few years. In older patients they may be reserved for adjunctive use when levodopa is no longer effective alone or cannot be tolerated. They are sometimes useful in reducing ‘off’ periods with levodopa and in ameliorating fluctuations in mobility in the later stages of the disease.

Apomorphine is a potent dopamine agonist, but must be given parenterally and with an antiemetic. Although this restricts its use, it has a role in stabilising patients who suffer unpredictable ‘on-off’ effects. It is also used in the differential diagnosis of parkinsonism. Transdermal patches containing rotigotine, another dopamine agonist, are available for use as monotherapy in the treatment of early-stage Parkinson's disease in some countries.

Antimuscarinics are considered to have a weak antiparkinsonian effect compared with levodopa. They may reduce tremor but have little effect on bradykinesia. They may be of use alone or with other drugs in the initial treatment of patients with mild symptoms, especially when tremor is pronounced, or later as an adjunct to levodopa, such as in patients with refractory tremor or dystonias. Antimuscarinic adverse effects, particularly cognitive impairment, occur frequently and can limit their use. However, some antimuscarinic effects can ameliorate complications associated with Parkinson's disease; dry mouth may be an advantage in patients with sialorrhoea. There appear to be no important differences in the efficacy of antimuscarinics for Parkinson's disease but some patients may tolerate one drug better than another. Those commonly used for Parkinson's disease include benzatropine, orphenadrine, procyclidine, and trihexyphenidyl.

Amantadine is a weak dopamine agonist with some antimuscarinic activity although its activity as an antagonist of N-methyl-D-aspartate may also have a beneficial effect in Parkinson's disease. It has mild antiparkinsonian effects compared with levodopa but is relatively free from adverse effects. It can improve bradykinesia as well as tremor and rigidity although only a small proportion of patients derive much benefit. It is used similarly to antimuscarinics in early disease when symptoms are mild, but tolerance to its effects can occur rapidly.

If symptoms are mild, drug therapy may not be required in the early stages of the disease. When symptoms become troublesome but are still relatively mild amantadine or an antimuscarinic may be started; antimuscarinics are useful when tremor predominates but are generally more suitable for younger patients and in drug-induced rather than idiopathic parkinsonism. Some have begun treatment with selegiline immediately, but there have been doubts over whether it has a neuroprotective effect, as postulated, and also over long-term safety. There is no consensus on when to start dopaminergic treatment or whether to begin with levodopa or a dopamine agonist. For most patients treatment with levodopa eventually becomes necessary, but many neurologists delay initial treatment with levodopa because of the increased risk of motor complications. New patients, especially younger patients, therefore often begin treatment with a dopamine agonist, with levodopa reserved for the elderly, the frail, or those with intercurrent illness or more severe symptoms.

When levodopa does become necessary, the usual practice is to start with small doses, together with a peripheral dopa-decarboxylase inhibitor, and increase slowly to a dose which reduces disability to an acceptable level. Variations in response and diminishing effectiveness over the years necessitate careful adjustment of the size and form of the dose and the dosage schedule.

Pramipexole was proposed in late 2009 as an early-stage treatment alternative to Levodopa.

Recently there has been a consensus that younger Parkinson's patients first be treated with dopamine agonists while older patients should be given levodopa.

Selegiline and rasagiline reduce the symptoms of Parkinson's disease by inhibiting monoamine oxidase-B (MAO-B). MAO-B breaks down dopamine secreted by the dopaminergic neurons, so inhibiting it will result in inhibition of the breakdown of dopamine. Metabolites of selegiline include L-amphetamine and L-methamphetamine (not to be confused with the more notorious and potent dextrorotary isomers). This might result in side effects such as insomnia. Use of L-dopa in conjunction with selegiline has increased mortality rates that have not been effectively explained. Another side effect of the combination can be stomatitis. One report raised concern about increased mortality when MAO-B inhibitors were combined with L-dopa; however subsequent studies have not confirmed this finding. Unlike other non selective monoamine oxidase inhibitors, tyramine-containing foods do not cause a hypertensive crisis.

Dementia is another neurodegenerative condition that is characterized by a progressive decline in cognitive function which may be due to damage or disease in the brain beyond what might be expected from normal aging. Areas particularly affected include memory, attention, judgement, language and problem solving. Dementia typically begins gradually and worsens progressively over several years due to neuronal degeneration of the brain and causing gradual but irreversible loss of function. The causes of dementia depend on the age at which symptoms begin. In the elderly population (usually defined in this context as over 65 years of age), a large majority of cases of dementia are caused by Alzheimer's disease, vascular dementia or both. Dementia with Lewy bodies is another fairly common cause, which again may occur alongside either or both of the other causes

Several agents are currently used for the treatment of dementia.

Acetylcholinesteraseinhibitors: Tacrine (Cognex), donepezil (Aricept), galantamine (Razadyne), and rivastigmine (Exelon) are approved by the United States Food and Drug Administration (FDA) for treatment of dementia induced by Alzheimer's disease. They may be useful for other similar diseases causing dementia such as Parkinsons or vascular dementia.

The medications introduced for the treatment of dementia were the cholinesterase inhibitors (ChEI) in 1997. Since this time most clinicians and probably most patients would consider the cholinergic drugs, donepezil, galantamine and rivastigmine, to be the first line pharmacotherapy for mild to moderate Alzheimer's disease. The individual drugs have slightly different pharmacological profiles, but they all work by inhibiting the breakdown of acetylcholine, an important neurotransmitter associated with memory, by blocking the enzyme acetylcholinesterase. The most that these drugs could achieve is to modify the manifestations of Alzheimer's disease. N-methyl-D-aspartate Blockers. Memantine (Namenda) is a drug representative of this class. It can be used in combination with acetylcholinesterase inhibitors. Amyloid deposit inhibitors: Minocycline and Clioquinoline, antibiotics, may help reduce amyloid deposits in the brains of persons with Alzheimer's disease.

Antidepressant drugs: Depression is frequently associated with dementia and generally worsens the degree of cognitive and behavioral impairment. Antidepressants effectively treat the cognitive and behavioral symptoms of depression in patients with Alzheimer's disease, but evidence for their use in other forms of dementia is weak.

Anxiolytic drugs: Many patients with dementia experience anxiety symptoms. Although benzodiazepines like diazepam (e.g Valium) have been used for treating anxiety in other situations, they are often avoided because they may increase agitation in persons with dementia and are likely to worsen cognitive problems or are too sedating. Buspirone (Buspar) is often initially tried for mild-to-moderate anxiety. There is little evidence for the effectiveness of benzodiazepines in dementia, whereas there is evidence for the effectivess of antipsychotics (at low doses).

Selegiline, a drug used primarily in the treatment of Parkinson's disease, appears to slow the development of dementia. Selegiline is thought to act as an antioxidant, preventing free radical damage. However, it also acts as a stimulant, making it difficult to determine whether the delay in onset of dementia symptoms is due to protection from free radicals or to the general elevation of brain activity from the stimulant effect.

Three areas of cognitive impairment associated with dementia have been targeted for research in recent years, this is in part due to the enormous cost to society of caring for a growing aging and increasingly dependent population thus the improvement in function or even the slowing of the illness process will result in a considerable reduction in the burden of this care. Subjective Cognitive Impairment (SCI) is a mild and variable condition with an identified nonspecific cognitive impairment. Mild Cognitive Impairment (MCI) is a diagnosis given to individuals who have cognitive impairments beyond that expected for their age and education, but which do not interfere significantly with their daily activities. It is considered to be the boundary or transitional stage between normal aging and dementia and is seen as a risk factor for Alzheimer's disease. The third is Alzheimer's Type Dementia (ATD) and associated dementias which represent the most severe and end-stage presentation of cognitive impairment in the elderly. MCI can present with a variety of symptoms, but when memory loss is the predominant symptom it is termed “amnesic MCI” and is frequently seen as a risk factor for Alzheimer's disease. Studies suggest that these individuals tend to progress towards probable Alzheimer's disease at a rate of approximately 10% to 15% per year. Studies suggest that individuals with MCI tend to progress towards probable Alzheimer's disease with an 80% conversion rate within five years of onset. SCI is considered a prodromal MCI condition, and may last up to approximately 15 years. Deterioration in social cognition and symptoms of higher executive dysfunction are commonly cited as potential sensitive markers of later progression to more significant cognitive impairment. Therefore both SCI and MCI may provide the best opportunities for clinical intervention, aiming for the possible re-direction or least delay of the eventual loss of function.

There is no proven treatment or therapy for mild cognitive impairment. As MCI may represent a prodromal state to clinical Alzheimer's disease, treatments proposed for Alzheimer's disease, such as antioxidants and cholinesterase inhibitors, may be useful. In fact, several potential treatments are currently under investigation. Two drugs used to treat Alzheimer's disease have been explored in particular, for their ability to effectively treat MCI or prevent/slow down the progress towards full Alzheimer's disease. Rivastigmine failed to stop or slow progression to Alzheimer's disease or improve cognitive function for individuals with MCI, and Donepezil showed only minor, short-term benefits and was associated with significant side effects. Recently, there have been favourable reports regarding Colostrinin, which confirm the drug offers a viable treatment for MCI.

Furthermore, compliance with pharmacotherapy is a long standing and difficult problem with individuals both with and without attention and concentration impairment. Reduced compliance affects and potentially limits the efficacy of all interventions, frequently being the most limiting factor in providing sustained psychotherapeutic benefit. For example, the 12 month compliance rate for use of psychostimulants in adults is approximately 33%. The core areas of impairment appear to be in the sequence and organisation of thoughts. This is seen clinically with adults with a diagnosis of attention deficit hyperactivity disorder [(ADHD); a DSM-IV-TR disorder as described in the Fourth Edition of the Diagnostic and Statistical Manual of Mental Disorders (American Psychiatric Association, 2000), and Snyder, Nussbaum, & Robins (Eds.), 2006, ibid (especially Box 2) and Weiss & Murray, 2003] and most commonly treated with psychostimulants. The initial and at times dramatic improvement frequently gives way to a returning disorganisation, and non-adherence with medication and an eventual cessation of treatment. Unless there is a concurrent improvement in the automatic and effortless ability to process social information, the gains in motivation provided by the stimulant will inevitably wane resulting in the associated return of symptoms.

Spina Bifida (SB) is the most common and severely disabling neurogenetic disorder with prevalence studies indicating rates of; 18 out of every 100 000 births in the U.S. (Holmbeck, Essner, Kelly, Friedman, DeLucia, Zebracki & Jandasek, 2010), 1 per 1000 in Europe and around 6 per 1000 live births in parts of India and China (Oakeshott, Hunt, Poulton & Reid, 2009). The aetiology of SB remains complex and produces considerable phenotypic variability (Fletcher & Brei, 2010) that involves cognition, behaviour, adaptation and neurologic dysfunction on multiple organ systems. Major organ affects are related to paralysed or weakened lower extremities that often misguide placement of this condition into an orthopaedic category with the associated ambulatory effects (Fletcher & Brei). Urinary and bowel incontinence problems are an unfortunate and common feature in addition to hydrocephalus and learning disorders (Holmbeck et al).

Recent decades have seen prevalence rates and the survival prognosis of SB alter substantially although this has not necessarily translated into universal behaviour modification. Dietary fortification including the taking of supplements containing folic acid prenatally has assisted the decline of neural tube deficits although much support and education is required internationally for this to produce the desired prevention rates (Fletcher & Brei). Prior to the 1960's the predicted life outcome was poor for individuals with SB and the welcomed developments in neurosurgical intervention witnessed the prognosis to survival at 1 year rise significantly from 20% to 80% (Oakeshott et al). Longitudinal cohort studies have provided researchers, clinicians and families affected by SB with important information relating to mean age of survival that in the Oakeshott study was 40 years of age, in addition to greater understanding of health and disability concerns, impact on learning, living independently and connecting socially. An extension of this has been to more closely examine the influences or impediments to academic learning or social engagement for an individual coping with this severe spinal dysraphism or split spine and determine if other conditions co-exist with SB.

One study of children affected by spina bifida meningomyelocele with hydrocephalus (SBH) which accounts for 95% of SB and 80-90% having CSF shunting, has confirmed the incidence of ADHD at 31%, significantly higher than the prevalence figures of ADHD in the general population of approximately 17% and depicted in this study, the ADHD prevalence rate was 5% in the comparison group and 8% in the normative sample (Burmeister, Hannay, Copeland, Fletcher, Boudousquie & Dennis, 2005). Furthermore, the authors in this study posited that behaviours associated with distractibility, lack of focus and disorganisation are more associated with SBH than hyperactive, impulsive behaviours.

Furthermore, a growing body of literature has recognised the important and interacting components of social skills (Fletcher & Brei; Holmbeck et al, 2003; Rose & Holmbeck, 2007) in individuals with SB. In a preadolescent age group (8-9 years of age) SB sample, it was revealed that these particular children demonstrated social immaturity and passisivity, were less inclined to have social contacts outside of school, indicated an increased dependence on adults for guidance, reduced likely hood for scholastic success, were less physically active, had a reduced ability to make independent decisions and with an increased tendency to illustrate attention and concentration difficulties (Holmbeck et al, 2003). In a separate study (Rose & Holmbeck, 2007), it was revealed that attention and executive deficits were predictive of social adjustment difficulties with a meditational analysis inferring that neurocognitive deficits mediated the association between spina bifida status and social adjustment difficulty.

The higher executive and social functioning deficits identified in SB may also reflect similar cognitive difficulties in other conditions which have been successfully treated with Ultra low dose phenyloin. We have noted in the following example of a similar pattern of improvements SB to these other conditions.

Chronic neck pain affecting the cervical vertebrae region can impose episodes of distress and disability for an individual and severely limit a multitude of lifestyle factors. The burden of continuous non-malignant pain is one of the main precursors for seeking medical care and pain literature has indicated the important association of poor quality of life outcomes and depression symptomatology (Townsend, Sletten, Bruce, Rome, Leutze, & Hodgson, 2005; Demyltennaere et al, 2007). Furthermore, research into chronic pain and comorbidity has urged clinicians to be aware of a wider spectrum of mental disorders that may co-occur in greater frequency with chronic pain such as anxiety and mood disorders in addition to alcohol dependency (Demyltennaere et al). Inferring both the history of pain and prognostic value, the term chronic pain is defined as pain that persists beyond the normal time of healing and in the category of non-malignant pain, three months is a common medically agreed upon time frame to distinguish acute phases of pain to chronic phases (Von Korff & Dunn, 2008; Young Casey, Greenberg, Nicassio, Harpin & Hubbard, 2008). Although clinical debate continues regarding the suitability of chronic pain classification systems, the duration of pain model for determining chonicity indicates tissue damage is associated with acute pain signalling and chronic pain stems from central and peripheral sensitization of pain that has sustained beyond the period when nociceptive inputs have diminished (Von Korff & Dunn).

The prevalence of chronic neck pain is often implicated by the presence of chronic back pain. Regions of the back and neck invoking debilitating pain responses are among the most frequently described pain conditions in general populations of the developed world (Demyltennaere et al). The ramifications of such commonly occurring pain symptoms from individuals can be extensive in terms of health facility utilization, increasing expenditure and reduced employment participation as indicated by a large population based survey revealing that 29% of adults had experienced back or neck pain in the previous month with 50% of this group reporting chronic pain (Webb in Von Korff, 2005). Several spinal pain studies (Makela et al, 1991; Bovin et al, 1994; Rajalaetal et al, 1995) from Europe and the U.S. have indicated that a within a prior 12 month time period, the prevalence rates for neck pain were between 12% and 34% (as cited in Demyttenaere et al).

Determining a direct patho-aetiology of neck pain can be assisted by the reference to broad categories such as non-degenerative and degenerative neck pain with the former category including a suspected differential diagnosis of fracture, subluxation or dislocation (trauma), infection, neoplastic and vascular and the latter degenerative diagnosis including axial neck pain, cervical radiculopathy and cervical myelopathy (Rogers, 2010).

According to a study by Young Casey et al involving an aetiological model of chronic pain and disability in patients, the existence of baseline depressive symptoms and pain permanence beliefs were strong predictors of chronic disability often leading to passive coping and avoidance and hence exacerbating the disability.

The ability to control noxious stimuli, either from pain or sound can be seen as the ability to successfully and automatically without effort habituate, or ignore the unwanted stimuli. This can be done temporarily with effort but is very exhausting and cannot be sustained for any length of time. Similarly we have noted that many patients are more able to ignore sound which was previously intrusive and distracting on commencement of the ultra low dose phenyloin. The invasive and distressing nature of the cervical pain was reduced with unexpected efficacy and rapidity.

Antiepileptic medications are commonly used for the control of chronic and neuropathic pain. Furthermore, it has been has been described that sodium channel blockers such as phenyloin exhibit analgesic effects (Lai, Porreca, Hunter, & Gold, 2004). Certain antiepileptic medication can be used effectively to control pain and some of these medications, such as pregabalin, have approved indications for use in treatment in neuropathic pain. However, the rapidity of the response at less than 2.5% of the usual therapeutic doses would not be anticipated based on the current knowledge of phenyloin.

Despite the fact that there are many agents that can be used alone or in combination to treat neurological disorders, there is a need for improving treatment of these diseases. These improvements may relate to the efficacy of the treatment (e.g. in terms of patient outcomes such as quality of life and amelioration of symptoms), reduction or elimination of side-effects and/or the cost of treatment, although without limitation thereto.

According to a first aspect of the invention, there is provided an anti-epileptic agent for use in the treatment of a neurological disorder other than epilepsy, characterised in that the anti-epileptic agent is the sole active agent and that the daily dose of the anti-epileptic is less than 20% of the minimum daily dose which is effective for mood stabilisation or treatment of epileptic symptoms.

In the context of the present invention, neurological disorders include disorders associated with impaired, abnormal or reduced cognitive processing, particularly that which enables higher executive functioning. Neurodegenerative conditions, such as dementia, Parkinson's disease, Alzheimer's disease and Huntington's disease are included within the neurological disorders of the present invention. In addition to the neurodegenerative conditions mentioned above, the neurological disorders of the present invention include learning disorders, reading disorders, acquired brain injury, autism, tardive dyskinesia (TD), attention deficit hyperactivity disorder (ADHD), spina bifida (SB), chronic pain, post traumatic stress disorder (PTSD), schizophrenia and visual acuity/fatigue.

Thus, the neurological disorders of the invention are suitably selected from the group consisting of neurodegenerative conditions, such as dementia, Parkinson's disease, Alzheimer's disease and Huntington's disease; learning disorders; reading disorders; acquired brain injury; autism (including autistic spectrum disorders or ASD); tardive dyskinesia; attention deficit hyperactivity disorder (ADHD); spina bifida; chronic pain; post traumatic stress disorder (PTSD); schizophrenia; and visual acuity/fatigue.

In an embodiment of the invention, the neurological disorder excludes bipolar disorder and/or ADHD. Additionally or alternatively, it may exclude neurodegenerative disorders.

Phenyloin (5,5-diphenylhydantoin), which has been in use for 60 years, is still an important antiepileptic drug. Its primary mechanism of action is modulation of the sustained repetitive firing of neurones by direct inhibition and blockage of voltage-gated sodium channels in the neuronal cell membrane, and by delay of cellular reactivation. The plasma protein binding of phenyloin is normally between 90% and 95%. The drug is rapidly distributed from the blood to the tissues and is almost completely metabolized in the liver. The plasma phenyloin concentration normally reaches the steady-state level within 1-2 weeks. The half-life of phenyloin is less than 20 h in low doses, but is prolonged in high doses.

Based on this description and mechanism of action one would not consider it likely that a dose of less than 2.5% of the therapeutic dose for epilepsy/bipolar mood disorder would be effective. Particularly in the context of the high plasma protein binding the rapid action and response noted in the oral and sublingual routes would not be expected to be inconsistent with the above pharmacokinetics.

The dose of the anti-epileptic agent is less than 20% of the minimum daily dose which is effective for mood stabilisation or treatment of epileptic symptoms, for example, less than 10%. In certain embodiments, the dose of the anti-epileptic agents is a low dose, such as for example, less than 7.5% or less than 5% of the minimum daily dose which is effective for mood stabilisation of epilepsy or epileptic symptoms. In further embodiments, the dose of the anti-epileptic is an ultra low dose, such as for example, less than 2.5%, less than 2%, less than 1.5% or less than 1% of the minimum daily dose which is effective for mood stabilisation of epilepsy or epileptic symptoms. Suitably, the amount of the anti epileptic equates to an ultra low dose.

Suitably, the daily dose of the anti-epileptic agent is greater than 0.001% of the minimum daily dose which is effective for mood stabilisation or treatment of epileptic symptoms.

In low dose embodiments, the dose may be administered daily, at multiple time each day or at pre-determined times during the week. Thus, the anti-epileptic agent may be administered one, two, three, four, five or six times per week, rather than daily or more than once per day. Where the medicament is administered in accordance with a dosage regimen of less than one dose per day (e.g. where the medicament is administered one, two, three, four, five or six times per week), the medicament may be formulated as a controlled release or a sustained release pharmaceutical composition.

According to a second aspect of the invention, there is provided a pharmaceutical composition comprising a sub-therapeutic dose of an anti-epileptic agent as the sole active agent within the composition, together with a pharmaceutically acceptable carrier, diluent and/or excipient, wherein the sub-therapeutic dose is less than 20% of the minimum daily dose of the anti-epileptic agent which is effective for mood stabilisation or treatment of epileptic symptoms.

The amount of the anti-epileptic agent present in the composition may be such that the composition is able to deliver the desired daily dose as discussed above. Thus, for compositions adapted to be delivered as a single daily dose, the amount of the anti-epileptic agent present may be as discussed above. However, for compositions adapted to be administered more than once per day, the amount of the anti-epileptic present in the composition would be correspondingly lower.

It has been found that transdermal administration, particularly via the oral mucosa, is an efficient mode of administration for the compositions according to the second aspect of the invention. An example of transdermal delivery via the oral mucosa is a sub-lingual composition. Thus, transdermal compositions, including in particular compositions adapted to be delivered across the oral mucosa, such as powders, capsules, tablets, lozenges or pastilles are suitable forms for delivering the anti-epileptic agent. Alternative transdermal compositions include patches or dressings which are adapted to be secured (e.g. temporarily adhered) to the skin of a patient. Thus, an adhesive patch containing a composition accordingly to the invention forms an embodiment of the invention.

In embodiments where the intended route of administration is oral, the pharmaceutical composition may be formulated as an immediate release formulation or it may be formulated as a controlled release formulation, sustained release formulation or a delayed release formulation.

Furthermore, the composition may be a combination of immediate release and controlled release, sustained release and/or delayed release. For example, the composition may comprise an immediate release layer or compartment and a controlled release, sustained release and/or delayed release layer or compartment.

According to a third aspect of the invention, there is provided a method of treating a neurological disorder other than epilepsy in a subject in need thereof, including the step of administering to the subject an anti-epileptic agent as the sole active agent, wherein the daily dose of the anti-epileptic agent is less than 20% of the minimum daily dose which is effective for mood stabilisation or treatment of epileptic symptoms.

The skilled person will appreciate that the amount of the anti-epileptic agent used in the method according to the third aspect of the invention will be as discussed in connection with the first aspect of the invention. Additionally, the skilled person will appreciate that the neurological disorder may be a neurological disorder as defined or mentioned herein.

In addition to use of the anti-epileptic as a sole active, it may be used in combination with a second active selected from a stimulant, an anti-Parkinson's agent, an analgesic and an acetylcholinesterase inhibitor. Thus, according to a fourth aspect of the invention, there is provided a combination of:
(a) an anti-epileptic agent; and
(b) an active selected from a stimulant, an anti-Parkinson's agent, an analgesic and an acetylcholinesterase inhibitor
   for use in the treatment of a neurological disorder other than epilepsy, characterised in that the daily dose of the anti-epileptic agent is less than 2.5% of the minimum daily dose which is effective for mood stabilisation or treatment of epileptic symptoms.

The skilled person will appreciate that the fourth aspect of the invention uses an ultra low dose of the anti-epileptic agent, which is less than 2.5%, such as less than 2%, less than 1.5% or less than 1% of the minimum daily dose which is effective for mood stabilisation or treatment of epileptic symptoms. As with the first aspect of the invention, the amount of the anti-epileptic agent present in the combination is suitably more than 0.001% of the minimum daily dose which is effective for mood stabilisation or treatment of epileptic symptoms.

Similar to the second aspect of the invention, there is also provided a fifth aspect of the invention which provides a pharmaceutical composition comprising:
(a) a sub-therapeutic dose of an anti-epileptic agent;
(b) an active selected from a stimulant, an anti Parkinson's agent, an analgesic and an acetylcholinesterase inhibitor; and
(c) a pharmaceutically acceptable carrier, diluent and/or excipient,
   wherein the sub-therapeutic dose is less than 2.5% of the minimum daily dose which is effective for mood stabilisation or treatment of epileptic symptoms.

As with the second aspect of the invention, the amount of the anti-epileptic agent present in the composition may be such that the composition is able to deliver the desired daily dose as discussed above. Thus, for compositions adapted to be delivered as a single daily dose, the amount of the anti-epileptic agent present may be as discussed above. However, for compositions adapted to be administered more than once per day, the amount of the anti-epileptic present in the composition would be correspondingly lower. Similarly, the composition may in a transdermal form (e.g. formulated for sub-lingual administration or as a patch) as discussed above.

The pharmaceutical composition may be formulated as an immediate release formulation or it may be formulated as a controlled release formulation, sustained release formulation or a delayed release formulation. Furthermore, the composition may be a combination of immediate release and controlled release, sustained release and/or delayed release. For example, the composition may comprise an immediate release layer or compartment and a controlled release, sustained release and/or delayed release layer or compartment.

According to a sixth aspect of the invention, there is provided a method of treating a neurological disorder other than epilepsy in a subject in need thereof, including the step of administering to the subject a combination of (a) an anti-epileptic agent, and (b) an active selected from a stimulant, an anti-Parkinson's agent, an analgesic and an acetylcholinesterase inhibitor, wherein the daily dose of the anti-epileptic agent is less than 2.5% of the minimum daily dose which is effective for mood stabilisation or treatment of epileptic symptoms.

In an embodiment of the invention as defined in any of the aspects detailed above, the anti-epileptic agent may be selected from brivaracetam, carbamazepine, clobazam, clonazepam, dantrolene, eslicarbazepine acetate, ethosuximide, ezogabine, felbamate, gabapentin, ghrelin, lacosamide, lamotrigine, levetiracetam, oxcarbazepine, phenobarbital, phenyloin, pregabalin, primidone, retigabine, rufinamide, safinamide, seletracetam, talampanel, tiagabine, tizanidine, topiramate, valproate, vigabatrin, zonisamide, 2-(1H-Benzotriazol-1-yl)-N′-[substituted]acetohydrazides, 4-aminopyridine, benzodiazepines, barbiturates and sedative hypnotics.

In embodiments of the invention where a stimulant is included, the stimulant may be selected from Adrafinil, Amantadine, Armodafinil, Carphedon, Modafinil, 4-Fluoroamphetamine, 4-Fluoromethamphetamine, 4-Methylmethcathinone, 4-MTA, α-PPP, Amphechloral, Amphetamine, Dextroamphetamine, Adderall, Amphetaminil, Benzphetamine, Bupropion, Cathinone, Chlorphentermine, Clobenzorex, Clortermine, Cypenamine, Diethylpropion, Dimethoxyamphetamine, Dimethylamphetamine, Dimethylcathinone, Diphenyl prolinol, Ephedrine, Epinephrine, Ethcathinone, Ethylamphetamine, Fencamfamine, Fenethylline, Fenfluramine, Fenproporex, Feprosidnine, Furfenorex, Levomethamphetamine, Lisdexamfetamine, L-lysine-d-amphetamine, MDMA, Mefenorex, Methamphetamine, Methcathinone, Methoxyphedrine, Methylone, Octopamine, Parahydroxyamphetamine, PMA, PMEA, PMMA, PPAP, Phendimetrazine, Phenmetrazine, Phentermine, Phenylephrine, Phenylpropanolamine, Prolintane, Propylamphetamine, Pseudoephedrine, Selegiline, Synephrine, Tenamphetamine, Xylopropamine; piperazines, BZP, MeOPP, MBZP, mCPP, 2C-B-BZP, Tropanes, Brasofensine, CFT, Cocaethylene, Cocaine, Dimethocaine, Lometopane, PIT, PTT, RTI-121, Tesofensine, Troparil, WF-23, WF-33, Cholinergics, Arecoline, Cotinine, Convulsants, Bicuculline, Gabazine, Pentetrazol, Picrotoxin, Strychnine, Thujone; Phenylaminooxazoles, 4-Methyl-aminorex, Aminorex, Clominorex, Fenozolone, Fluminorex, Pemoline, Thozalinone, Amineptine, Bemegride, BPAP, Clenbuterol, Clofenciclan, Cyclopentamine, Cyprodenate, Desoxypipradrol, Ethylphenidate, Ethamivan, Gilutensin, GYKI-52895, Hexacyclonate, Indanorex, Indatraline, Isometheptene, Mazindol, MDPV, Mesocarb, methylphenidate, Dexmethylphenidate, Naphthylisopropylamine, Nikethamide, Nocaine, Nomifensine, Phacetoperane, Phthalimidopropiophenone, Pipradrol, Prolintane, Propylhexedrine, Pyrovalerone, Tuamine, Vanoxerine, Yohimbine, Zylofuramine, Deanol, Diethylaminoethanol, Dimefline Hydrochloride, Etilamfetamine Hydrochloride, Fencamfamin Hydrochloride, Fenetylline Hydrochloride, Fenfluramine Hydrochloride, Fenproporex Hydrochloride, Lobeline Hydrochloride, Pentetrazol, and Propylhexedrine.

In further embodiments of the invention where a second active is present (i.e. according to the fourth, fifth or sixth aspects), the anti-Parkinson's agent may be selected from apomorphine, benserazide, benzatropine, bromocriptine, cabergoline, carbidopa, clozapine, domperidone, entacapone, levodopa, lisuride, orphenadrine, pergolide, piribedil, pramipexole, procyclidine, quetiapine, rasagiline, rivastigmine, ropinirole, rotigotine, selegiline, tolcapone, trihexyphenidyl, a dopamine agonist, a dopamine decarboxylase inhibitor, a catechol O methyl transferase (COMT) enzyme inhibitor, a monoamine oxidase-B inhibitor and an N-methyl-D-aspartate blocker.

In still further embodiments of the invention according to the fourth, fifth or sixth aspects, the acetylcholinesterase inhibitor is selected from tacrine, donepezil, galantamine and rivastigmine.

The skilled person will appreciate that the term “anti-epileptic agent” can include two or more different components or compounds which are effective in the treatment of epilepsy or epilepsy-related symptoms, or it can comprise a single active component or compound. In the case where the agent comprises two or more different active components, the daily dose for each of the components is less than the specified amount of that component which is effective for mood stabilisation of epilepsy or epileptic symptoms.

Unless defined otherwise, all technical and scientific terms used herein have the same meaning as commonly understood by one of ordinary skill in the art (e.g. in pharmaceutical chemistry and medicine, including psychiatry).

Unless contraindicated or noted otherwise, in these descriptions and throughout this specification, the terms “a” and “an” mean one or more, the term “or” means and/or.

Unless defined otherwise, all technical and scientific terms used herein have the same meaning as commonly understood by one of ordinary skill in the art (e.g. in pharmaceutical chemistry).

By “comprising” is meant including, but not limited to, whatever follows the word “comprising”. Thus, use of the term “comprising” indicates that the listed elements are required or mandatory, but that other elements are optional and may or may not be present.

By “consisting of” is meant including, and limited to, whatever follows the phrase “consisting of:” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, “subject” or “individual” or “patient” refers to any subject for whom or which therapy is desired, and generally refers to the recipient of the therapy to be practiced according to the invention. The subject can be any vertebrate, but will suitably be a mammal. If a mammal, the subject will suitably be a human, but may also be a domestic livestock, laboratory subject or pet animal. The subject is most suitably a human adult, child or infant, who is or has been the subject of treatment, observation or experiment.

As used herein, unless the context demands otherwise, the term “treat,” “treating,” or “treatment” means to counteract a medical condition (e.g., a neurological disorder) to the extent that the medical condition is improved according to clinically acceptable standard(s). For example, “to treat a neurological disorder” means to improve the disorder or relieve symptoms of the particular disorder in a patient, wherein the improvement and relief are evaluated with a clinically acceptable standardised test (e.g., a patient self-assessment scale) and/or an empirical test. “Treat,” “treating,” or “treatment” as used herein also includes prophylactic treatment unless the context requires otherwise.

As used herein, the term “active agent”, “active” or “agent” means any substance which can affect any physical or biochemical properties of a biological system, pathway, molecule, or interaction relating to an organism, including but not limited to animals and humans. In particular, as used herein, agents include but are not limited to any substance intended for diagnosis, cure, mitigation, treatment, or prevention of disease in humans or other animals, or to otherwise enhance physical or mental well-being of humans or animals. Examples of biologically active molecules include, but are not limited to, peptides, proteins, enzymes and small molecule drugs. Classes of active agents that are suitable for use with the methods and compositions described herein include, but are not limited to, drugs, prodrugs, radionuclides, imaging agents, polymers and the like.

Certain agents, biologically-active molecules and other active compounds according to this invention may exist as enantiomers. Where they possess two or more chiral centers, they may additionally exist as diastereomers. It is to be understood that all such isomers and mixtures thereof are encompassed within the scope of the present invention. Furthermore, some of the crystalline forms for the agents or compounds may exist as polymorphs and as such are intended to be included in the present invention. In addition, some of the agents or compounds may form solvates with water (i.e., hydrates) or common organic solvents, and such solvates are also intended to be encompassed within the scope of this invention.

It will also be appreciated that the term “agent”, whether in the context of an anti-epileptic agent or an agent for treating a neurological disease, disorder or condition, may be in the form of a pharmaceutically effective or acceptable salt.

As used herein, the terms “co-therapy” and “combination therapy” shall mean treatment of a subject in need thereof by administering one or more anti-epileptic agent(s) and one or more agents for treating a neurological, disease, disorder or condition by any suitable means, simultaneously, sequentially, separately or in a single pharmaceutical formulation or combination. When administered in separate dosage forms, the number of dosages administered per day for each component may be the same or different. The anti-epileptic agent(s) and one or more agents for treating a neurological, disease, disorder or condition may be administered via the same or different routes of administration.

As hereinbefore described, the invention provides sole or combination therapy of a neurological disease, disorder or condition, wherein an anti-epileptic agent alone or a combination of one or more anti-epileptic drugs and one or more further active therapeutically effective in the treatment of a neurological disease disorder or condition is administered to a subject to thereby treat the neurological disease, disorder or condition, Non-limiting examples of neurological diseases, disorders or conditions include learning disorders, reading disorders, acquired brain injury, tardive dyskinesia, subjective cognitive impairment (SCI), mild cognitive impairment (MCI), dementia (including Alzheimer's Type Dementia (ATD)), Parkinson's disease, Huntington's disease, pervasive development and communication disorders, autism (including ASD), attention deficit hyperactivity disorder (ADHD), spina bifida (SB), chronic pain, post traumatic stress disorder (PTSD), schizophrenia and visual acuity/fatigue.

As used herein, unless otherwise noted, the term “anti-epileptic agent” and the abbreviation “AED” will be used interchangeably with the terms “anti-convulsant agent”, “anticonvulsant” “anti-epileptic mood stabilizer”, “mood stabilizer”, and “anti-epileptic” and refer to an agent capable of treating, inhibiting or preventing seizure activity or ictogenesis and/or achieving mood stabilisation when the agent is administered to a subject or patient.

While not wishing to be bound by any particular theory, it is believed that the exact chemical class of AED is not determinative of the utility of any specific AED in the compositions and methods of the invention. Rather, it is the efficacy of AEDs in treatment of epileptic, pre-epileptic, or ictogenic events, convulsions, mood stabilization that identifies the relevant compounds or agents useful within the invention. Thus, AEDs of diverse chemical classes are useful and relevant (with suitable adjustments of dose) according to the invention.

In embodiments of the invention, the amount of anti-epileptic agent(s), when used as the sole active, is less than 20% of the daily dose of anti-epileptic agent typically effective in mood stabilization or in treating epileptic symptoms. In particular embodiments, the amount of anti-epileptic agent is less than 10%, 5%, 2.5%, 2%, 1.5% or 1% of the daily dose of anti-epileptic agent typically effective in mood stabilization or in treating epileptic symptoms. Suitably, the daily dose of the AED is at least 0.001% of the daily dose of anti-epileptic agent typically effective in mood stabilization or in treating epileptic symptoms.

Particular examples of AEDs include sodium valproate (sodium di-n-propylacetic acid) and derivatives thereof (valproic acid, valproate pivoxil, semi-sodium valproate, divalproex, valproylamides such as valpromide, Depakene, Depakote, Depakote ER), tiagabine, ethosuximide, zonisamide, carbamazepine, oxcarbazepine, lamotrigine, tiagabine, gabapentin, pregabalin, phenyloin, primidone, phenobarbitone, phenobarital, topiramate, diazepam and related compounds, and levetiracetam.

In particular embodiments the AED is selected from the group consisting of brivaracetam, carbamazepine, clobazam, clonazepam, ethosuximide, felbamate, gabapentin, lacosamide, lamotrigine, levetiracetam, oxcarbazepine, phenobarbital, phenyloin, pregabalin, primidone, retigabine, rufinamide, safinamide, seletracetam, talampanel, tiagabine, topiramate, valproate, vigabatrin, zonisamide, benzodiazepines, barbiturates and sedative hypnotics.

Particularly suitable AEDs are sodium valproate and derivatives thereof, tiagabine, topiramate, carbamazepine, oxcarbazepine, ethotoin, phenyloin, gabapentin, pregabalin, and rufinamide. In another embodiment, the anti-convulsant or anti-epileptic agent(s) is selected from the group consisting of carbamazepine, clobazam, clonazepam, ethosuximide, felbamate, gabapentin, lamotrigine, levetiracetam, oxcarbazepine, phenobarbital, phenyloin, pregabalin, primidone, retigabine, rufinamide, talampanel, tiagabine, topiramate, valproate, vigabatrin and zonisamide.

In another embodiment, the AED, anti-convulsant or anti-epileptic agent(s) is selected from the group consisting of carbamazepine, lamotrigine, phenobarbital, phenyloin, topiramate, valproate and zonisamide. Suitably, the anti-convulsant or anti-epileptic agent(s) is selected from the group consisting of carbamazepine, gabapentin, lamotrigine, levetiracetam, oxcarbazepine, phenyloin, pregabalin, rufinamide, valproate and topiramate. More suitably, the anti-convulsant or anti-epileptic is selected from the group consisting of gabapentin, lamotrigine, levetiracetam, pregabalin, rufinamide, valproate and topiramate. In a further embodiment, the anti-epileptic is selected from the group consisting of valproate, rufinamide, topiramate, and phenyloin.

In particular embodiments, examples of anti-convulsant or anti-epileptic agents include, but are not limited to, the following, described non-exclusively by either mode of action or chemical class:
(a) AMPA antagonists such as AMP-397, E-2007, NS-1209, talampanel, perampanel, and the like;
(b) Benzodiazepines such as diazepam, lorazepam, clonazepam, clobazam, clorazepate, midazolam, nimetazepam, nitrazepam, temasepam, and the like;
(c) Barbiturates such as phenobarbital, amobarbital, methylphenobarbital, primidone, Barbexaclone sodium, metharbital, pentobarbital, and the like;
(d) Valproates (including fatty acid derivatives) such as valproic acid, valproate semisodium, valpromide, divalproex, valnoctamide, and the like;
(e) GABA related agents such as gabapentin (2-[1-(aminomethyl)cyclohexyl]acetic acid), pregabalin ((S)-3-(aminomethyl)-5-methylhexanoic acid), vigabatrin, and the like;
(f) AEDs such as losigamone, retigabine, rufinamide (1-[(2,6-difluorophenyl)methyl]triazole-4-carboxamide), SPD-421 (DP-VPA), T-2000, XP-13512, and the like;
(g) Iminostilbenes such as carbamazepine, oxcarbazepine, eslicarbazepine acetate and the like;
(h) Hydantoins such as phenyloin sodium, Phenyloin, mephenyloin, fosphenyloin sodium, ethotoin, and the like;
(h) NMDA antagonists such as harkoseride, and the like;
(i) Sodium channel blockers such as BIA-2093, CO-102862, lamotrigine, and the like;
(j) Succinimides such as methsuximide, ethosuximide, and the like;
(k) Carboxylic acids such as tiagabine, and the like;
(l) AEDS such as acetazolamide, clomthiazole edisilate, zonisamide, felbamate, topiramate, tiagabine, levetiracetam, briveracetam, GSK-362115, GSK-406725, ICA-69673, CBD cannabis derivative, isovaleramide (NPS-1776), RWJ-333369 (carisbamate), safinamide, seletracetam, soretolide, stiripentol, valrocemide, and the like;
(m) oxazolidinediones such as trimethadione, paramethadione, ethadione and the like;
(n) succinimides such as ethosuximide, phensuximide, mesuximide, and the like;
(o) pyrrolidines such as levetiracetam, and the like;
(p) sulphonamides, such as acetazolamide, methazolamide, zonisamide, sultiame, and the like;
(q) aminobutyric acids and the like;
(r) sulfamate-substituted monosaccharides such as topiramate (2,3:4,5-Bis-O-(1-methylethylidene)-beta-D-fructopyranose sulfamate)), and the like;
(s) carboxamides such as carbamazepine, oxcarbazepine, rufinamide, and the like;
(t) aromatic allylic alcohols such as stiripentol, and the like;
(u) ureas such as phenacemide, pheneturide, and the like;
(v) phenyltriazines such as lamotrigine, and the like;
(w) carbamates such as emylcamate, felbamate, meprobamate, and the like;
(x) pyrrolidines such as brivaracetam, levetriacetame, nefiracetam, selectracetam, and the like;
(y) eugenols such as (4-Allyl-2-Methoxyphenol), phenyleugenol, benzyleugenol, and phenylethyleugenol;
(z) epalons such as ganaxolone and the like; and
(za) neuroleptics such as ghrelin and the like.

In one embodiment, the mood stabiliser is a gamma-aminobutyric acid (GABA) enhancer, i.e. a GABAergic agent.

In further examples, a variety of AEDs have been described in the art and useful as anti-epileptics and mood stabilizers. For example, those mentioned in the following published patents or patent applications describe, in relation to the agent they disclose, both suitable methods for their preparation and doses for their administration. These publications are herein incorporated by reference.

EP-0021121-A discloses a group of 3,5-diamino-6-(substituted phenyl)-1,2,4-triazines which are active in the treatment of central nervous system (CNS) disorders, for example in the treatment of epilepsy. One such triazine is 3,5-diamino-6-(2,3-dichlorophenyl)-1,2,4-triazine which is alternatively called lamotrigine. EP-0372934-A discloses pyrimidine compounds useful in the treatment of CNS disorders. Example 18 of EP-0372934-A discloses 2,4-diannino-5-(2,3-dichlorophenyl)-6-fluoromethyl pyrimidine.

WO 97/09317 discloses the R(−) enantiomer of this compound, R(+2,4-diamino-5-(2,3-dichlorophenyl)-6-fluoromethylpyrimidine, substantially free of the corresponding S(+) enantiomer. WO98/38174 discloses pyrazine derivatives, including rufinamide, useful in the treatment of CNS disorders such as epilepsy. WO99/32462 relates to a triazine compound which is useful in the treatment of central nervous system (CNS) diseases and disorders, i.e. the compound 5-amino-6-[2,3,5-trichlorophenyl]-1,2,4-triazine and pharmaceutically acceptable derivatives thereof. WO00/12488 relates to pyrazine compounds useful in the treatment of CNS diseases and resulting disorders.

As used herein “one or more agents effective in the treatment of a neurological condition or disorder” includes any agent useful in the treatment of neurodegenerative disorders including Parkinson's disease and dementia.

Non-limiting examples of agents useful in treating Parkinson's disease include apomorphine, benserazide, benzatropine, bromocriptine, cabergoline, carbidopa, clozapine, domperidone, entacapone, levodopa, lisuride, orphenadrine, pergolide, piribedil, pramipexole, procyclidine, quetiapine, rasagiline, rivastigmine, ropinirole, rotigotine, selegiline, tolcapone and trihexyphenidyl,

In one embodiment for treating Parkinson's disease, the agent is L-dopa or levodopa.

In another embodiment for treating Parkinson's disease, the agent is a dopamine agonist. Non-limiting examples include bromocriptine, pergolide, pramipexole, ropinirole, piribedil, apomorphine, cabergoline, lisuride and pramipexole.

In an additional or alternative embodiment for treating Parkinson's disease the agent is a dopamine decarboxylase inhibitor. Non-limiting examples include carbidopa and benserazide

In further additional or alternative embodiment for treating Parkinson's disease, the agent inhibits the catechol O methyl transferase (COMT) enzyme. Non-limiting examples include tolcapone and entacapone.

In a yet further additional or alternative embodiment for treating Parkinson's disease, the agent is a monoamine oxidase-B inhibitor, Non-limiting examples include selegiline and rasagiline.

In a yet still further additional or alternative embodiment for treating Parkinson's disease, the agent is a N-methyl-D-aspartate blocker. Non-limiting examples include Memantine (Namenda).

In embodiments relating to treatment of dementia, the agent is preferably an acetylcholinesterase inhibitor. Non-limiting examples include tacrine, donepezil, galantamine and rivastigmine.

As used herein, unless otherwise noted, the term “stimulant”, “psychostimulant” or “psychostimulant agent” and the terms “central nervous system stimulant” and “CNS stimulant” will be used interchangeably and refer to an agent capable of producing an increase or enhancement in psychomotor activity. However, and as known to those of skill in the art and as herein defined, the terms “psychostimulant” and “CNS stimulant” as used herein do not refer to agents such as caffeine and nicotine, which are not considered to be psychostimulants, at least because they do not enhance locomotor behavior in rodents (Sulzer, D., et al. Prog. Neurobio. 75(6): 406-433).

A large number of pyschostimulants are known in the art and suitable for use in the invention. While not wishing to be bound by any particular theory, it is believed that the exact chemical class of psychostimulant is not determinative of the utility of any specific psychostimulant in the compositions and methods of the invention. Rather, it is the efficacy of psychostimulants in increasing or enhancing psychomotor activity that is encompassed by the invention. Thus, psychostimulants of diverse chemical classes are equally useful and relevant (with suitable adjustments of dose) in combination with similarly diverse classes of AEDs within the scope of the invention. Indeed, clinical examples are provided that demonstrate effectiveness and relevance of diverse classes of psychostimulants in combination with diverse classes of AEDs.

Psychostimulants useful for the compositions on the invention include, but are not limited to: methylphenidate (Ritalin) administered at about 0.01 to about 2.5 mg/kg/day; dextroamphetamine (Dexedrine) administered at about 0.07 to about 1.5 mg/kg/day; amphetamine (Adderall) administered at about 0.05 to about 1.5 mg/kg/day; and pemoline (Cylert) administered at about 0.1 to about 2.0 mg/kg/day.

Examples of psychostimulants with use in the invention include the class of compounds identifiable as amphetamines. The term “amphetamine” as understood by those of skill in the art, typically contains an alpha-methyl-phenethyl-amine motif. Exemplary amphetamines are amphetamine, methamphetamine, and dextroamphetamine or “dexamphetamine”. Dextroamphetamine or “D-amphetamine” or “dexamphetamine” is the dextrorotary (D) stereoisomer of amphetamine. Amphetamines in pharmaceutical form include, for example, dextroamphetamine sulphate (Dexamin™, Dextrostat™, Dexadrine™), dexamphetamine or mixed amphetamine salts (Adderall XR™)) and pemoline (Cylert™)).

Methylphenidate is typically formulated for pharmaceutical use as the hydrochloride (e.g. Ritalin™ Ritaline LA™, Focalin™, Concerta™, Methylin, Attenta™, Lorentin™, Daytrana™, Tranquilyn™, Equasym™, Riphenidate™, Rubifen™, Metadate CD™ Biphentin™). Methylphenidate is described in U.S. Pat. No. 2,957,880 and Biphentin™ in Canadian Patents 2355854 and 2355644. Though not technically an amphetamine, methylphenidate functions in a similar way in the CNS or brain. Methylphenidate typically has a relatively short duration of action (2 to 4 hours). Hence, slow release or continual release formulations or methods of delivery have been developed, e.g. Concerta™ and the transdermal patch, marketed as Daytrana™. Further examples of slow or controlled release formulations are known in the art, for example as described in published US patent application no. 2007/0059349.

Typical doses for these medications are described in Wilens and Dodson, 2004, Clin. Psychiatry 65: 1301-1313 (methylphenidate—juveniles: 0.6 to 1.0 mg/kg/day; adults 20 to 100 mg per day, amphetamine—juveniles: 0.3 to 1.5 mg/kg/day; adults 10 to 70 mg/day, pemoline—juveniles: 1.0 to 3.0 mg/kg/day; adults 75 to 150 mg/day).

Additional examples useful in the invention include: Eugeroics such as Adrafinil, Armodafinil, Carphedon, Modafinil; Phenethylamines such as 4-Fluoroamphetamine, 4-Fluoromethamphetamine, 4-Methylmethcathinone, 4-MTA, α-PPP, Amphechloral, Amphetamine (Dextroamphetamine, Adderall), Amphetaminil, Benzphetamine, Bupropion, Cathinone, Chlorphentermine, Clobenzorex, Clortermine, Cypenamine, Diethylpropion, Dimethoxyamphetamine, Dimethylamphetamine, Dimethylcathinone, Diphenyl prolinol, Ephedrine, Epinephrine, Ethcathinone, Ethylamphetamine, Fencamfamine, Fenethylline, Fenfluramine, Fenproporex, Feprosidnine, Furfenorex, Levomethamphetamine, Lisdexamfetamine (Vyvance™) (L-lysine-d-amphetamine), MDMA, Mefenorex, Methamphetamine, Methcathinone, Methoxyphedrine, Methylone, Octopamine, Parahydroxyamphetamine, PMA, PMEA, PMMA, PPAP, Phendimetrazine, Phenmetrazine, Phentermine, Phenylephrine, Phenylpropanolamine, Prolintane, Propylamphetamine, Pseudoephedrine, Selegiline, Synephrine, Tenamphetamine, Xylopropamine; piperazines such as BZP, MeOPP, MBZP, mCPP, 2C-B-BZP; Xanthines such as Aminophylline, Paraxanthine, Theobromine, Theophylline; Tropanes such as Brasofensine, CFT, Cocaethylene, Cocaine, Dimethocaine, Lometopane, PIT, PTT, RTI-121, Tesofensine, Troparil, WF-23, WF-33; Cholinergics such as Arecoline, Cotinine; Convulsants such as Bicuculline, Gabazine, Pentetrazol, Picrotoxin, Strychnine, Thujone; Phenylaminooxazoles such as 4-Methyl-aminorex, Aminorex, Clominorex, Fenozolone, Fluminorex, Pemoline, Thozalinone; Others such as Amantadine, Amineptine, Bemegride, BPAP, Clenbuterol, Clofenciclan, Cyclopentamine, Cyprodenate, Desoxypipradrol, Ethylphenidate, Ethamivan, Gilutensin, GYKI-52895, Hexacyclonate, Indanorex, Indatraline, Isometheptene, Mazindol, MDPV, Mesocarb, methylphenidate, Dexmethylphenidate, Naphthylisopropylamine, Nikethamide, Nocaine, Nomifensine, Phacetoperane, Phthalimidopropiophenone, Pipradrol, Prolintane, Propylhexedrine, Pyrovalerone, Tuamine, Vanoxerine, Yohimbine, Zylofuramine, Deanol, Diethylaminoethanol, Dimefline Hydrochloride, Etilamfetamine Hydrochloride, Fencamfamin Hydrochloride, Fenetylline Hydrochloride, Fenfluramine Hydrochloride, Fenproporex Hydrochloride, Lobeline Hydrochloride, Pentetrazol, Propylhexedrine.

Combinations of two or more pyschostimulants may be used. References to all pyschostimulant described herein include pharmaceutically acceptable salts thereof, as appropriate, and slow release and extended release formulations, as well as prodrugs of the listed active agents. An example of such a prodrug is lisdexamfetamine (L-lysine-d-amphetamine).

Therapeutic combinations of the invention comprise, in addition to an anti-epileptic agent, one or more of a stimulant, an anti-Parkinson's agent, an analgesic or a cholinesterase inhibitor (hereinafter referred to as “the further active”), effective in combination to provide enhanced treatment of one or more neurological diseases, conditions or disorders, or symptoms or another underlying cause of the symptom(s), in comparison with either agent alone. The therapeutically effective amount of co-therapy comprising administration of one or more of the further active and an anti-epileptic agent would include an amount of the further active and the anti-epileptic agent that, when taken together or sequentially, have a combined effect that is therapeutically effective.

In certain embodiments of therapeutic combinations and combination formulations or dosage regimes, particularly those utilized in particular methods of the invention described herein, the dose administered of the anti-epileptic is less than 2.5% of the minimum daily dose which is effective for mood stabilization, controlling seizures or mania. This means that the dose administered is below the dose range that would be administered to epileptics and individuals with bipolar disorders to achieve mood stabilization, control of seizures or control of mania, as appropriate. As mentioned above, the use of such sub-therapeutic dosages is advantageous for the treatments described herein.

Therapeutically effective dosage levels and dosage regimens for the anti-epileptic agents disclosed herein may be readily determined by one of ordinary skill in the art. For example, therapeutic dosage amounts and regimens for pharmaceutical agents approved for sale are publicly available, for example as listed on packaging labels, in standard dosage guidelines, in standard dosage references such as the Physician's Desk Reference (Medical Economics Company or online at http://www.pdrel.com) and other sources.

In the case of sodium valproate, the product information for Epilim (Sanofi-Aventis) states that, for the treatment of mania (e.g. bipolar disorder) in adults, control of symptoms typically occurs within the range of 1,000 to 2,000 mg/day, (i.e. approximately 14 to 29 mg/kg/day based on a 70 kg adult). In the case of carbamazepine, a typical dose for treating epileptic seizures is in the range of from 400 to 800 mg/day. In the case of topiramate, the target dose for controlling epileptic seizures is between 100 to 500 mg/day.

By contrast, in relation to sodium valproate (and derivatives thereof), a sub-therapeutic dose with respect to mood stabilization is considered in this context to be less than 200 mg/day or 2.86 mg/kg/day (based on an adult weighing 70 Kg), a suitable dose being less than 150 mg/day, less than 100 mg/day, less than 50 mg/day, or less than 25 mg/day. The minimum dose is typically at least 1 mg/day, such as at least 2.5, 5 or 10 mg/day. The doses are expressed both independently of patient weight and based on patient weight since minimum and maximum doses can apply.

Typically, the mg/kg/day is more commonly applied in relation to children whereas the total mg/day may be more appropriate for adults. The skilled person is able to calculate an appropriate amount for a child based on the suggested adult dose. A number of different techniques are available for such conversions and some of these are discussed in Calculation of Drug Dosage and Body Surface Area of Children; British Journal of Anaesthesia; 1997; 78: 601-605, for example.

These dosages in relation to sodium valproate and derivatives thereof represent, at the upper end, less than 20% of the lower end of the normal therapeutic dose range for mood stabilisation or treating epileptic symptoms, and at the lower end, about 0.001% of the normal therapeutic dose range for treating epilepsy or bipolar disorder. In certain embodiments, the upper range of the dose is less than 2.5%, namely, less than 25 mg/day. These dosages can be used as a guide for calculating the relative dosages of other mood stabilizers that would constitute a sub-therapeutic dose.

For example, in the case of carbamazepine, a suitable sub-therapeutic dose is in the range of from 1 to less than 80 mg/day, such as more than 2, 5 or 7.5 mg/day but less than 80 or 50 mg/day. In certain embodiments, the upper level of carbamazepine is less than 2.5%, namely less than 10 mg/day.

In the case of topiramate, a suitable sub-therapeutic dose is in the range of from 0.5 to less than 20 mg/day, such as at least 1 or 1.5 mg/day but less than 15 or 10 mg/day. In certain embodiments, the upper level of topiramate is less than 2.5% of the normal dose to treat epilepsy, namely less than 2.5 mg/day

In the case of phenyloin, a suitable sub-therapeutic dose is in the range of from 1 mg to less than 40 mg/day, such as at least 1.5 or 2 mg/day but less than 40 or 30 mg/day. In certain embodiments, the upper level of phenyloin is less than 2.5% of the normal dose to treat epilepsy, namely less than 5 mg/day.

In the case of pregabalin, a suitable sub-therapeutic dose is in the range of from 1 to less than 60 mg/day, such as more than 2 or 4 mg/day but less than 60 or 50 mg/day. In certain embodiments, the upper level of pregabalin is less than 2.5% of the normal dose to treat epilepsy, namely less than 7.5 mg/day.

In the case of rufinamide a suitable sub-therapeutic dose is in the range of from 1 to less than 80 mg/day, such as more than 2 or 4 mg/day but less than 80 or 70 mg/day. In certain embodiments, the upper level of rufinamide is less than 2.5% of the normal dose to treat epilepsy, namely less than 10 mg/day.

Preferably, the sub-therapeutic dose is less than 20%, such as less than 10% of the minimum dose that would be administered to epileptics to achieve mood stabilization, control of seizures or control of mania, as appropriate. In certain embodiments, the sub-therapeutic dose is an ultra low dose which is less than 2.5% of the minimum dose that would be administered to epileptics and individuals with bipolar disorders to achieve mood stabilization, control of seizures or control of mania, as appropriate.

The following is a non-limiting, exemplary list of some AED with their usual minimum mood stabilisation or anti-convulsant doses to illustrate a calculation of an initial sub-therapeutic AED dose. A sub-therapeutic dose for mood stabilization in the context of the present invention is therefore less than 20% of the minimum dosages listed below for each particular agent e.g. for Ethotoin, a sub-therapeutic dose is less than 200 mg/day. The minimum dose to be administered in the context of the present invention is suitably at least 0.01, 0.05, 0.1, 0.5 or 1% of the minimum therapeutic dose for mood stabilization listed below, e.g. in the case of Ethotoin, at least 1, 5 or 10 mg/day. In the case of rufinamide, a dose within the sub-therapeutic range for antiepileptic therapy or mood stabilization is less than 20% of the minimum therapeutic dose for mood stabilization listed below, i.e. less than 80 mg/day.

| | Minimum typical dose/day |
|---|---|
| | effective for mood |
| | stabilisation or treatment of |
| Agent | epileptic symptoms or events |
| Aminoglutethimide | 125 mg |
| Barbexaclone | 200 mg in divided doses |
| Belcamide | 1 mg |
| Brivaracetam | 100 mg |
| Carbamazepine | 400 mg |
| Clobazam | 5 mg/kg daily |
| Clonazepam | 1 mg |
| Ethadione | 1000 mg |
| Ethosuximide | 1000 mg |
| Ethotoin | 1000 mg |
| Felbamate | 1200 mg |
| Fosphenytoin Sodium | 10 mg/kg |
| Gabapentin | 900 mg |
| Lacosamide | 200 mg |
| Lamotrigine | 100 mg |
| Levetiracetam | 1000 mg |
| Losigamone | 1500 mg |
| Mephenytoin | 200 mg |
| Methoin | 1000 mg |
| Methsuximide | 300 mg |
| Oxcarbazepine | 600 mg |
| Paramethadione | 300 mg |
| Perampanel | 2 mg |
| Phenacemide | 500 mg |
| Pheneturide | 600 mg |
| Phensuximide | 1000 mg |
| Phenytoin | 200 mg |
| Pregabalin | 300 mg |
| Primidone | 750 mg |
| Retigabine | 600 mg |
| Rufinamide | 400 mg |
| Sultiame | 200 mg |
| Tiagabine Hydrochloride | 30 mg |
| Topiramate | 100 mg |
| Trimethadione | 900 mg |
| Vigabatrin | 1000 mg |
| Zonisamide | 200 mg |

In some embodiments, the dosage administered of AED is sub-therapeutic for mood stabilization for the entire, or at least substantially the entire, treatment period. In other words, it is suitable that the dosage administered of mood stabiliser does not exceed the maximum stated sub-therapeutic dosages described above throughout the treatment.

Particularly suitable combinations of AEDs and the further active: (i) one or more of sodium valproate and derivatives thereof, topiramate, carbamazepine, oxcarbazepine, phenyloin, gabapentin or pregabalin; together with either (ii) one or more psychostimulants, (iii) one or more cholinesterase inhibitors for treating dementia or (iv) one or more of levodopa and dopamine agonists. For combination formulations comprising an AED and a further active, the intended daily dose of AED may range from 0.001% to less than 2.5% of the minimum dosages for treatment of epilepsy or mood disorder for each particular AED, while the normal, recommended amount of the further active is used.

Particular doses for particular combinations may be created using a matrix formed by rows of AED doses with columns of further active doses. For example, an entry of (20 mg of AED, 30 mg of further active) in a matrix denotes 20 mg of AED and 30 mg of further active compounded as, for example, a single tablet or unit dose. Such a dose may be formulated or effective as a single, daily dose, or may be repeated a number of times in a day, for example to result in a total daily dose of 80 mg of AED and 120 mg of further active.

The units of measure of each agent may be divided as convenient into steps of 0.01, 0.5, 1.0, 2.0, 5.0 mg and the like. The units are not constrained by any particular step value and all possible values between the minimum and maximum doses for each agent are contemplated. Thus, the dimensions of the matrix row relevant to any particular AED are formed by its minimum and maximum contemplated doses along with the desired step values. Similarly, the matrix column dimensions are formed by the minimum and maximum contemplated doses of further active along with the desired step values. To include two or more AEDs or further active in a combination the matrix dimensions are increased by the addition of a dimension corresponding to the further agent. Hence, a 3 dimensional matrix would list all contemplated combination of three active agents. All combination unit doses and pharmaceutical compositions so described are within the scope of the invention.

AEDs, alone or in combination with a further active, may be administered in the form of a pharmaceutical composition, which further comprises a pharmaceutically acceptable carrier, diluent and/or excipient.

In a particular aspect, the invention provides a pharmaceutical composition comprising, in combination, one or a plurality of anti-epileptic agents, and either one or a plurality of further active, or pharmaceutically acceptable salts thereof.

In another particular aspect, the invention provides a pharmaceutical kit comprising a first pharmaceutical composition comprising (i) one or a plurality of anti-epileptic agents or a pharmaceutically acceptable salt thereof, together with a pharmaceutically acceptable carrier or diluent and (ii) a second pharmaceutical composition comprising one or a plurality of further active together with a pharmaceutically acceptable carrier, diluent or excipient.

The kit according to the invention may include a starter pack adapted for titration of the composition to the desired amount for a patient; and a maintenance pack adapted to maintain the dose of the composition at the pre-determined amount.

Examples of routes of administration for which the pharmaceutical composition may be suitable include, but are not limited to, oral, intravenous (iv), intramuscular (inn), subcutaneous (sc), transdermal (including via the oral mucosa), and rectal. Compositions may also be administered directly to the nervous system including, but not limited to, intracerebral, intraventricular, intracerebroventricular, intrathecal, intracisternal, intraspinal or peri-spinal routes of administration by delivery via intracranial or intravertebral needles or catheters with or without pump devices. The further active and the anticonvulsant or anti-epileptic agent(s) may be administered according to simultaneous or alternating regimens, at the same or different times during the course of the therapy, concurrently in divided or single forms.

Pharmaceutical compositions containing one or more of the agents described herein can be prepared by intimately mixing the compound or compounds with a pharmaceutical carrier, diluent and/or excipient according to conventional pharmaceutical compounding techniques.

As used herein, “pharmaceutically acceptable carrier” includes any material which, when combined with an active ingredient of a composition, allows the ingredient to retain biological activity and without causing disruptive reactions in the subject. Examples include, but are not limited to, any of the standard pharmaceutical carriers such as a phosphate buffered saline solution, water, emulsions such as oil and water emulsion, and various types of wetting agents. Preferred diluents for aerosol or parenteral administration are phosphate buffered saline or normal (0.9%) saline. Compositions comprising such carriers are formulated by well known conventional methods (see, for example, Remington's Pharmaceutical Sciences, Chapter 43, 14th Ed., Mack Publishing Col, Easton Pa. 18042, USA).

The carrier may take a wide variety of forms depending upon the desired route of administration (e.g., oral, parenteral). Thus, for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

Transdermal preparations typically include an adhesive patch which is adapted to be temporarily adhered to the skin.

For use in medicine, the salts of the agents of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid.

Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following: acids including acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, .alpha.-oxo-glutaric acid, glycolic acid, hipuric acid, hydrobromic acid, hydrochloric acid, (+)-L-lactic acid, (.+-.)-DL-lactic acid, lactobionic acid, maleic acid, (−)-L-malic acid, malonic acid, (.+-.)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitric acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

Optionally, the oral solid dosage form includes a sustained release carrier that effects the sustained release of the AED, or both the AED and the further active when the dosage form contacts gastrointestinal fluid. The sustained release dosage form may comprise a multiplicity of substrates and carriers that include the agents. The substrates may comprise matrix spheroids or may comprise inert pharmaceutically acceptable beads that are coated with the agents. The coated beads are then preferably overcoated with a sustained release coating comprising the sustained release carrier. The matrix spheroid may include the sustained release carrier in the matrix itself, or the matrix may comprise a simple disintegrating or prompt release matrix containing the drugs, the matrix having a coating applied thereon which comprises the sustained release carrier. In yet other embodiments, the oral solid dosage form comprises a tablet core containing the agents within a normal or prompt release matrix with the tablet core being coated with a sustained release coating comprising the sustained release carrier. In yet further embodiments; the tablet contains the agents within a sustained release matrix comprising the sustained release carrier.

In yet further embodiments, the tablet contains the AED within a sustained release matrix, and the further active coated into the tablet as an immediate release layer.

In some embodiments of the invention, the pharmaceutical compositions containing the further active and AED agents set forth herein are administered orally. Such oral dosage forms may contain one or all of the agents in immediate or sustained release form. The oral dosage forms may be in the form of tablets, troches, lozenges, aqueous, solid or semi-solid solutions or mixtures, or oily suspensions or solutions, dispersible powders or granules, emulsions, multiparticulate formulations, syrups, elixirs, and the like.

In other embodiments, a pharmaceutical composition containing the AED(s) and further active can be administered in dosage form as a topical preparation, a solid state and or depot type transdermal delivery device(s), a suppository, a buccal preparation, sub-lingual preparation, or an inhalation formulation such as a controlled release particle formulation or spray, mist or other topical vehicle, intended to be inhaled or instilled into the sinuses.

The pharmaceutical compositions containing the agents set forth herein may alternatively be in the form of microparticles such as microcapsules, microspheres and the like, which may be injected or implanted into a human patient, or other implantable dosage forms known to those skilled in the art of pharmaceutical formulation.

For administration orally, the compounds may be formulated individually or in combination as sustained release preparations. If formulated individually, different release times or bioavailability may be afforded each active agent though they may ultimately be compounded or mixed together into one unit dose. Numerous examples of techniques for formulating sustained release preparations are described in the following references: U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; WO 98/47491; and U.S. Patent Application Publications 2005/0266078; 2008/0057123; 2008/0026070; 2008/00757769; and 2008/0031946, all of which are incorporated herein by reference.

As an example of how certain embodiments of the pharmaceutical compositions of this invention are prepared, one or more of the further active and one or more of the anticonvulsant or anti-epileptic agents are intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavouring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques.

For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredients necessary to deliver an effective dose as described herein.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above.

In the context of combination unit doses, a pharmaceutical composition comprising the active agents may be formulated with distinct halves or further subdivisions, each half or subdivision comprising primarily one agent. Scoring or pre-division of the halves or subdivisions thereby allow easy modulation of dose of each active agent.

The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

In an additional embodiment, one or more of the further active may be separately formulated or compounded, then coated or embedded in one or more of the anticonvulsant or anti-epileptic agents or formulations thereof. Alternatively, the anticonvulsant or anti-epileptic agents or formulations thereof may be embedded in or otherwise bound to the further active or their formulations. Thus, the two or more active agents may be compounded separately but ultimately provided together in one unit dose as a combination. Each, separately compounded agent may thus be provided in timed release, slow release, or other suitable formulation specifically advantageous to that agent, though ultimately provided as a single unit dose.

In particular embodiments, one or a plurality of AEDs alone, or AEDs in combination with a further active, may be administered in the form of a pharmaceutical composition, including but not limited to the particular pharmaceutical compositions hereinbefore described.

In view of the teachings of the invention, optimal dosages and schedules to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages. Where a subject of patient proves to be particularly sensitive to an agent or combination therapy, doses can be appropriately adjusted, or alternative choice of agent(s) made within the teaching of the invention.

One skilled in the art will recognize that a therapeutically effective dosage of the combinations of the present invention can include repeated doses within a prolonged treatment regimen that will yield clinically significant results. Advantageously, combinations of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen. The combinations may be administered through a single transdermal patch, or via subdivided transdermal patches or even separate transdermal patches, as may be desired.

Determination of effective dosages is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in vitro models. Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response).

It will be generally understood that therapeutic methods may be practiced preventatively to prophylactically treat a neurological disorder, or may be used to treat an existing, recurring or on-going neurological disorder. Prophylactic treatments may be appropriate where, for example, a subject has a genetic predisposition and/or family history of a neurological disorder.

In this regard, methods may further include, prior to administration of the anti-epileptic agent alone or in combination with the second active, determining whether said subject is, or may be, in need of prophylactic or therapeutic treatment for the neurological disorder. This step may be performed by clinical assessment, genetic testing or genetic counseling, alone or in combination.

Preferably, patients, subjects or individuals treated by the method may be adult, juvenile, adolescent, child or infant humans.

In one embodiment, the neurological disorder is associated with an impairment or deficiency in higher order executive functioning. The executive system is a theorized cognitive system in psychology that controls and manages other cognitive processes. It is also referred to as the executive function, supervisory attentional system, or cognitive control.

The concept is used by psychologists and neuroscientists to describe a loosely defined collection of brain processes which are responsible for planning, cognitive flexibility, abstract thinking, rule acquisition, initiating appropriate actions and inhibiting inappropriate actions, and selecting relevant sensory information.

Higher order executive functioning is thought to be heavily involved in handling novel situations outside the domain of some of our ‘automatic’ psychological processes that could be explained by the reproduction of learned schemas or set behaviors. Psychologists have outlined five types of situation where routine activation of behavior would not be sufficient for optimal performance:

(i) those that involve planning or decision making;

(ii) those that involve error correction or troubleshooting;

(iii) situations where responses are not well-learned or contain novel sequences of actions;

(iv) dangerous or technically difficult situations; and/or

(v) situations which require the overcoming of a strong habitual response or resisting temptation.

In another embodiment, the neurological disorder is not a developmental disorder or a disorder usually diagnosed in infancy, childhood or adolescences.

In yet another embodiment, the neurological disorder is a degenerative disorder. Examples of degenerative disorders include Mild Cognitive Impairment (MCI), Alzheimer's Disease, Amyotrophic Lateral Sclerosis, Corticobasal Degeneration, Creutzfeldt-Jakob Disease, Dementia with Lewy Bodies, Frontotemporal Dementia, Huntington's Disease, Progressive Supranuclear Palsy, Vascular Dementia, movement disorders such as Parkinson's disease, dementia associated with multiple sclerosis and motor neurone disease.

In still yet another embodiment, the neurological disorder is a psychotic disorder. Non-limiting examples are schizophrenia and psychotic disorders and/or behaviour resulting from causes including brain tumors, drug abuse with amphetamines, cocaine, cannabis, alcohol etc., brain damage (acquired or otherwise), bipolar disorder (manic depression), severe clinical depression, severe psychosocial stress, sleep deprivation, some focal epileptic disorders especially if the temporal lobe is affected, exposure to some traumatic event (e.g. violent death, road accident), abrupt or over-rapid withdrawal from certain recreational or prescribed drugs, neurological disorders, including: brain tumour, dementia with Lewy bodies, multiple sclerosis, sarcoidosis, Alzheimer's Disease and Parkinson's Disease.

In still yet another embodiment, the neurological disorder is associated with reduced adherence, or non-compliance, with a medication regime that includes the administration of a therapeutic agent other than, or in addition to, a psychostimulant. This embodiment in particular relates to long-time, multiple or complex medication regimes, such as those used in the treatment of hypertension, elevated cholesterol/lipids and diabetes (e.g. insulin). For example, compliance with long-term treatment for chronic asymptomatic conditions such as hypertension is on the order of 50%. (Loghman-Adham 2003.

In a further embodiment, the neurological disorder is an eating disorder. Non-limiting examples include Anorexia Nervosa and Bulimia Nervosa.

In other particular embodiments, treatment of dementia and sleep disorders are particularly suited to AED therapy without a further active, wherein two or more different AEDs are administered.

In certain embodiments, the present invention is particularly suited to treating a neurological disorder selected from the group consisting of: degenerative disorders and/or movement disorders such as Parkinson's disease, dementia and Mild Cognitive Impairment addiction; reduced adherence; eating disorders such as Anorexia Nervosa and Bulimia Nervosa; and personality disorders.

In other particular embodiments of the invention, the neurological disorder is selected from the group consisting of: Communication Disorders; Pervasive Development Disorders; and Anxiety Disorders.

Particular, non-limiting examples of these embodiments include neurological disorders that fall within the DSM-IV-TR classification: Communication Disorders (e.g. Expressive Language Disorder, Mixed Receptive-Expressive Language Disorder, Phonological Disorder, Stuttering, Communication Disorder NOS (=Not Otherwise Specified); Pervasive Development Disorders (Autistic Spectrum Disorders such as Autistic Disorder and Asperger's Disorder; Rett's Disorder, Childhood Disintegrative Disorder and Pervasive Developmental Disorder NOS); and Anxiety Disorders (e.g. Generalized Anxiety Disorder).

In view of the teachings of the invention, optimal dosages and schedules to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages. Where a subject of patient proves to be particularly sensitive to an agent or combination therapy, doses can be appropriately adjusted, or alternative choice of agent(s) made within the teaching of the invention.

The skilled person will appreciate that where a component is stated as being present in an amount below a defined threshold level, this means that the component is present in a measurable amount and that amount is less than the defined maximum.

### REFERENCE LIST

Adler, L. A., & Cohen, J. (2004). Diagnosis and evaluation of adults with attention deficit hyperactivity disorder. Psychiatry Clinic, 27, 187-214.
Arnott, W., Sali, L., & Copland, D. (2011). Impaired reading comprehension in schizophrenia: evidence for underlying phonological processing deficits. Psychiatry research, 187(1-2), 6-10. Elsevier Ireland Ltd. doi:10.1016/j.psychres.2010.11.025
Association, A. P. (2000). Diagnostic and Statistical Manual of Mental Disorders, Fourth Edition: DSM-IV-TR® (p. 943). American Psychiatric Pub. Retrieved from http://books.google.com/books?id=w_HajjMnjxwC&pgis=1
Bales, J., Wagner, A., & Kline, A. (2009). Persistent cognitive dysfunction after traumatic brain injury: A dopamine hypothesis. Neurosci Biobehav Rev., 33(7), 981-1003. doi:10.1016/j.neubiorev.2009.03.011. Persistent
Basso, A., & Scarpa, M. T. (1990). Traumatic aphasia in children and adults: a comparison of clinical features and evolution. Cortex; a journal devoted to the study of the nervous system and behavior, 26(4), 501-14. Retrieved from http://www.ncbi.nlm.nih.gov/pubmed/1706973
Bayly, P. V., Cohen, T. S., Leister, E. P., Ajo, D., Leuthardt, E. C., & Genin, G. M. (2005). Deformation of the human brain induced by mild acceleration. Journal of neurotrauma, 22(8), 845-56. doi:10.1089/neu.2005.22.845
Becker, T., & Kilian, R. (2006). Psychiatric services for people with severe mental illness across western Europe: what can be generalized from current knowledge about differences in provision, costs and outcomes of mental health care? Acta psychiatrica Scandinavica. Supplementum, (429), 9-16. doi:10.1111/j.1600-0447.2005.00711.x
Bell, V., Halligan, P. W., & Ellis, H. D. (2006). Explaining delusions: a cognitive perspective. Trends in cognitive sciences, 10(5), 219-26. doi:10.1016/j.tics.2006.03.004
Bentall, R. P., Fernyhough, C., Morrison, A. P., Lewis, S., & Corcoran, R. (2007). Prospects for a cognitive-developmental account of psychotic experiences. The British journal of clinical psychology/the British Psychological Society, 46(Pt 2), 155-73. doi:10.1348/014466506X123011
Betancur, C. (2011). Etiological heterogeneity in autism spectrum disorders: more than 100 genetic and genomic disorders and still counting. Brain research, 1380, 42-77. doi:10.1016/j.brainres.2010.11.078
Biederman, J., Faraone, S. V., Monuteaux, M. C., & Biederman, J., Faraone, S. V., & Monuteaux, M. (2002). Differential effect of environmental adversity by gender: Rutter's index of adversity in a sample of boys and girls with and without ADHD. American Journal of Psychiatry, 159(9), 1556-1563. American Psychiatric Assn. doi:10.1176/appi.ajp.159.9.1556
Blissitt, P. A. (2006a). Care of the critically ill patient with penetrating head injury. Critical care nursing clinics of North America, 18(3), 321-32. doi:10.1016/j.ccell.2006.05.006
Blissitt, P. A. (2006b). Care of the critically ill patient with penetrating head injury. Critical care nursing clinics of North America, 18(3), 321-32. doi:10.1016/j.ccell.2006.05.006
Borg, E., & Counter, S. A. (1989). The middle-ear muscles. Scientific American, 261(2), 74-80. Retrieved from http://www.ncbi.nlm.nih.gov/pubmed/2667133
Brady, K., Pearlstein, T., Asnis, G. M., Baker, D., Rothbaum, B., Sikes, C. R., & Farfel, G. M. (2000). Efficacy and safety of sertraline treatment of posttraumatic stress disorder: a randomized controlled trial. JAMA: the journal of the American Medical Association, 283(14), 1837-44. Retrieved from http://www.ncbi.nlm.nih.gov/pubmed/10770145
Bramlett, H. M., & Dietrich, W. D. (2007). Progressive damage after brain and spinal cord injury: pathomechanisms and treatment strategies. Progress in brain research, 161, 125-41. doi:10.1016/S0079-6123(06)61009-1
Brekke, J., Kay, D. D., Lee, K. S., & Green, M. F. (2005). Biosocial pathways to functional outcome in schizophrenia. Schizophrenia Research, 80(2-3), 213-225. Retrieved from http://www.ncbi.nlm.nih.gov/pubmed/16137859
Bremner, J. D., Mletzko, T., Welter, S., Quinn, S., Williams, C., Brummer, M., Siddiq, S., et al. (2005). Effects of phenyloin on memory, cognition and brain structure in post-traumatic stress disorder: a pilot study. Journal of psychopharmacology (Oxford, England), 19(2), 159-65. doi:10.1177/0269881105048996
Buckley, P. F., Miller, B. J., Lehrer, D. S., & Castle, D. J. (2009). Psychiatric comorbidities and schizophrenia. Schizophrenia bulletin, 35(2), 383-402. doi:10.1093/schbul/sbn135
Busch, R. M., McBride, A., Curtiss, G., & Vanderploeg, R. D. (2005). The components of executive functioning in traumatic brain injury. Journal of clinical and experimental neuropsychology, 27(8), 1022-32. doi:10.1080/13803390490919263
Cienfuegos, A., March, L., Shelley, A. M., & Javitt, D. C. (1999). Impaired categorical perception of synthetic speech sounds in schizophrenia. Biological psychiatry, 45(1), 82-8. Retrieved from http://www.ncbi.nlm.nih.gov/pubmed/9894579
Cohen, A. S., & Docherty, N. M. (2004). Affective reactivity of speech and emotional experience in patients with schizophrenia. Schizophrenia research, 69(1), 7-14. doi:10.1016/S0920-9964(03)00069-0
Committee on Child Abuse and Neglect. (2001). Shaken Baby Syndrome: Rotational Cranial Injuries Technical Report. PEDIATRICS, 108(1), 206-210. doi:10.1542/peds.108.1.206
Costa, E., & Guidotti, A. (1996). Benzodiazepines on trial: a research strategy for their rehabilitation. Trends in pharmacological sciences, 17(5), 192-200. Retrieved from http://www.ncbi.nlm.nih.gov/pubmed/8669126
Coyle, J. T., Tsai, G., & Goff, D. (2003). Converging evidence of NMDA receptor hypofunction in the pathophysiology of schizophrenia. Annals of the New York Academy of Sciences, 1003, 318-27. Retrieved from http://www.ncbi.nlm.nih.gov/pubmed/14684455
Cummings, J. L. (1993). Frontal-subcortical circuits and human behavior. Archives of neurology, 50(8), 873-80. Retrieved from http://www.ncbi.nlm.nih.gov/pubmed/8352676
Davids, E., K is, B., Specka, M., & Gastpar, M. (2006). A pilot clinical trial of oxcarbazepine in adults with attention-deficit hyperactivity disorder. Progress in neuropsychopharmacology biological psychiatry (Vol. 30, pp. 1033-1038).
Demb, J. B., Boynton, G. M., Best, M., & Heeger, D. J. (1998). Psychophysical evidence for a magnocellular pathway deficit in dyslexia. Vision research, 38(11), 1555-9. Retrieved from http://www.ncbi.nlm.nih.gov/pubmed/9747491
Erdmann, J. (2011). Broad collaborations bring new energy to autism therapeutics. Chemistry & biology, 18(2), 142-3. doi:10.1016/j.chembiol.2011.02.006
Faden, A., Demediuk, P., Panter, S., & Vink, R. (1989). The role of excitatory amino acids and NMDA receptors in traumatic brain injury. Science, 244(4906), 798-800. doi:10.1126/science.2567056
Faraone, S. V., Biederman, J., Spencer, T., Mick, E., Murray, K., Petty, C., Adamson, J. J., et al. (2006). Diagnosing adult attention deficit hyperactivity disorder: are late onset and subthreshold diagnoses valid? The American journal of psychiatry, 163(10), 1720-9; quiz 1859. doi:10.1176/appl.ajp.163.10.1720
Forde, I., Holloway, J., Healy, O., & Brosnan, J. (2011). A dyadic analysis of the effects of setting and communication partner on elicited and spontaneous communication of children with Autism Spectrum Disorder and typically developing children. Research in Autism Spectrum Disorders, 5(4), 1471-1478.
Freeman, D., Garety, P. A., Kuipers, E., Fowler, D., Bebbington, P. E., & Dunn, G. (2007). Acting on persecutory delusions: the importance of safety seeking. Behaviour research and therapy, 45(1), 89-99. doi:10.1016/j.brat.2006.01.014
Gomot, M., & Wicker, B. (2012). A challenging, unpredictable world for people with Autism Spectrum Disorder. International journal of psychophysiology: official journal of the International Organization of Psychophysiology, 83(2), 240-7. doi:10.1016/j.ijpsycho.2011.09.017
Granacher, R. P. (2007). Traumatic Brain Injury: Methods for Clinical and Forensic Neuropsychiatric Assessment (Google eBook) (p. 560). CRC Press.
Green, M F, Kern, R. S., Braff, D. L., & Mintz, J. (2000). Neurocognitive deficits and functional outcome in schizophrenia: are we measuring the “right stuff”? Schizophrenia bulletin, 26(1), 119-36. Retrieved from http://www.ncbi.nlm.nih.gov/pubmed/10755673
Green, Michael F. (2006). Cognitive impairment and functional outcome in schizophrenia and bipolar disorder. The Journal of clinical psychiatry, 67(10), e12. Retrieved from http://www.ncbi.nlm.nih.gov/pubmed/17107235
Guastella, A. J., Mitchell, P. B., & Dadds, M. R. (2008). Oxytocin increases gaze to the eye region of human faces. Biological psychiatry, 63(1), 3-5. doi:10.1016/j.biopsych.2007.06.026
Hardman, J. M., & Manoukian, A. (2002). Pathology of head trauma. Neuroimaging clinics of North America, 12(2), 175-87, vii. Retrieved from http://www.neuroimaging.theclinics.com/article/S1052-5149(02)00009-6/fulltext
Hashimoto, T., Volk, D. W., Eggan, S. M., Mirnics, K., Pierri, J. N., Sun, Z., Sampson, A. R., et al. (2003). Gene expression deficits in a subclass of GABA neurons in the prefrontal cortex of subjects with schizophrenia. The Journal of neuroscience: the official journal of the Society for Neuroscience, 23(15), 6315-26. Retrieved from http://www.ncbi.nlm.nih.gov/pubmed/12867516
Heimberg, C., Gur, R. E., Erwin, R. J., Shtasel, D. L., & Gur, R. C. (1992). Facial emotion discrimination: III. Behavioral findings in schizophrenia. Psychiatry research, 42(3), 253-65. Retrieved from http://www.ncbi.nlm.nih.gov/pubmed/1496057
Horan, W. P., & Blanchard, J. J. (2003). Emotional responses to psychosocial stress in schizophrenia: the role of individual differences in affective traits and coping. Schizophrenia research, 60(2-3), 271-83. Retrieved from http://www.ncbi.nlm.nih.gov/pubmed/12591589
Insel, T. R. (2010). Rethinking schizophrenia. Nature, 468(7321), 187-93. Nature Publishing Group. doi:10.1038/nature09552
Jackson, J. H. (1873). ON THE ANATOMICAL & PHYSIOLOGICAL LOCALISATION OF MOVEMENTS IN THE BRAIN. The Lancet, 101(2581), 232-235. doi:10.1016/50140-6736(02)63385-9
Javitt, D C, Doneshka, P., Grochowski, S., & Ritter, W. (1995). Impaired mismatch negativity generation reflects widespread dysfunction of working memory in schizophrenia. Archives of general psychiatry, 52(7), 550-8. Retrieved from http://www.ncbi.nlm.nih.gov/pubmed/7598631
Javitt, Daniel C., Shelley, A.-M., Silipo, G., & Lieberman, J. A. (2000). Deficits in Auditory and Visual Context-Dependent Processing in Schizophrenia. ARCH GEN PSYCHIATRY, 57, 1131-1137.
Jones, H. M., & Pilowsky, L. S. (2002). Dopamine and antipsychotic drug action revisited. The British journal of psychiatry: the journal of mental science, 181, 271-5. Retrieved from http://www.ncbi.nlm.nih.gov/pubmed/12356650
Kadhim, H. J., Duchateau, J., & Sébire, G. (2008). Cytokines and brain injury: invited review. Journal of intensive care medicine, 23(4), 236-49. doi:10.1177/0885066608318458
Kessler, R C, Sonnega, A., Bromet, E., Hughes, M., & Nelson, C. B. (1995). Posttraumatic stress disorder in the National Comorbidity Survey. Archives of general psychiatry, 52(12), 1048-60. Retrieved from http://www.ncbi.nlm.nih.gov/pubmed/7492257
Kessler, Ronald C, Adler, L., Barkley, R., Biederman, J., Conners, C. K., Demler, O., Faraone, S. V., et al. (2006). The prevalence and correlates of adult ADHD in the United States: results from the National Comorbidity Survey Replication. The American journal of psychiatry, 163(4), 716-23. doi:10.1176/appl.ajp.163.4.716
Kim, E. (2002). Agitation, aggression, and disinhibition syndromes after traumatic brain injury. NeuroRehabilitation, 17(4), 297-310. Retrieved from http://www.ncbi.nlm.nih.gov/pubmed/12547978
Kneisl, C. R., & Trigoboff, E. (2008). Contemporary Psychiatric-Mental Health Nursing (2nd Edition) (p. 1024). Prentice Hall. Retrieved from http://www.amazon.com/Contemporary-Psychiatric-Mental-Health-Nursing-Edition/dp/013243489X
Konradi, C., & Heckers, S. (2003). Molecular aspects of glutamate dysregulation: implications for schizophrenia and its treatment. Pharmacology & therapeutics, 97(2), 153-79. Retrieved from http://www.ncbi.nlm.nih.gov/pubmed/12559388
Kraus, M. F., Susmaras, T., Caughlin, B. P., Walker, C. J., Sweeney, J. A., & Little, D. M. (2007). White matter integrity and cognition in chronic traumatic brain injury: a diffusion tensor imaging study. Brain: a journal of neurology, 130(Pt 10), 2508-19. doi:10.1093/brain/awm216
Kuipers, E., Garety, P., Fowler, D., Freeman, D., Dunn, G., & Bebbington, P. (2006). Cognitive, emotional, and social processes in psychosis: refining cognitive behavioral therapy for persistent positive symptoms. Schizophrenia bulletin, 32 Suppl 1, S24-31. doi:10.1093/schbul/sbl014
Kumar, R., Husain, M., Gupta, R. K., Hasan, K. M., Haris, M., Agarwal, A. K., Pandey, C. M., et al. (2009). Serial changes in the white matter diffusion tensor imaging metrics in moderate traumatic brain injury and correlation with neuro-cognitive function. Journal of neurotrauma, 26(4), 481-95. doi:10.1089/neu.2008.0461
Kurtz, M. M. (2005). Neurocognitive impairment across the lifespan in schizophrenia: an update. Schizophrenia research, 74(1), 15-26. doi:10.1016/j.schres.2004.07.005
Kéri, S., Kiss, I., & Kelemen, O. (2009). Sharing secrets: oxytocin and trust in schizophrenia. Social neuroscience, 4(4), 287-93. doi: 10.1080/17470910802319710
Lahti, A. C., Weiler, M. A., Tamara Michaelidis, B. A., Parwani, A., & Tamminga, C. A. (2001). Effects of ketamine in normal and schizophrenic volunteers. Neuropsychopharmacology: official publication of the American College of Neuropsychopharmacology, 25(4), 455-67. doi:10.1016/50893-133X(01)00243-3
Lai, J., Porreca, F., Hunter, J. C., & Gold, M. S. (2004). Voltage-gated sodium channels and hyperalgesia. Annual review of pharmacology and toxicology, 44, 371-97. doi:10.1146/annurev.pharmtox.44.101802.121627
Laruelle, M., Abi-Dargham, A., van Dyck, C. H., Gil, R., D'Souza, C. D., Erdos, J., McCance, E., et al. (1996). Single photon emission computerized tomography imaging of amphetamine-induced dopamine release in drug-free schizophrenic subjects. Proceedings of the National Academy of Sciences of the United States of America, 93(17), 9235-40. Retrieved from http://www.pubmedcentral.nih.gov/articlerender.fcgi?artid=38625&tool=pmcentrez&render type=abstract Latalova, K., Prasko, J., Diveky, T., & Velartova, H. (2011). Cognitive impairment in bipolar disorder. Biomedical Papers, 155(1), 19-26. doi:10.5507/bp.155.2011.003
Lewis, D. a, Volk, D. W., & Hashimoto, T. (2004). Selective alterations in prefrontal cortical GABA neurotransmission in schizophrenia: a novel target for the treatment of working memory dysfunction. Psychopharmacology, 174(1), 143-50. doi:10.1007/s00213-003-1673-x
Lux, W. E. (2007). A neuropsychiatric perspective on traumatic brain injury. The Journal of Rehabilitation Research and Development, 44(7), 951-962. doi:10.1682/JRRD.2007.01.0009
Maas, A. I. R., Stocchetti, N., & Bullock, R. (2008). Moderate and severe traumatic brain injury in adults. Lancet neurology, 7(8), 728-41. doi:10.1016/S1474-4422(08)70164-9
Manuscript, A., Revheim, N., Butler, P. D., Schechter, I., Jalbrzikowski, M., Silipo, G., & Javitt, D. C. (2006). Reading impairment and visual processing deficits in schizophrenia. Schizophrenia research, 87(1-3), 238-45. doi:10.1016/j.schres.2006.06.022
Matson, J. L., Kozlowski, A. M., Worley, J. A., Shoemaker, M. E., Sipes, M., & Horovitz, M. (2011). What is the evidence for environmental causes of challenging behaviors in persons with intellectual disabilities and autism spectrum disorders? Research in developmental disabilities, 32(2), 693-8. doi:10.1016/j.ridd.2010.11.012
Mattson, A. J., & Levin, H. S. (1990a). Frontal lobe dysfunction following closed head injury. A review of the literature. The Journal of nervous and mental disease, 178(5), 282-91. Retrieved from http://www.ncbi.nlm.nih.gov/pubmed/2187053
Mattson, A. J., & Levin, H. S. (1990b). Frontal lobe dysfunction following closed head injury. A review of the literature. The Journal of nervous and mental disease, 178(5), 282-91. Retrieved from http://www.ncbi.nlm.nih.gov/pubmed/2187053
McCrea, M. A. (2007). Mild Traumatic Brain Injury and Postconcussion Syndrome: The New Evidence Base for Diagnosis and Treatment (Oxford Workshop Series: American Academy of Clinical Neuropsychology) (p. 224). Oxford University Press, USA. Retrieved from http://www.amazon.com/Traumatic-Brain-Injury-Postconcussion-Syndrome/dp/0195328299
McDonald, S., Flanagan, S., Rollins, J., & Kinch, J. (2003). TASIT: A new clinical tool for assessing social perception after traumatic brain injury. The Journal of head trauma rehabilitation, 18(3), 219-38. Retrieved from http://www.ncbi.nlm.nih.gov/pubmed/12802165
McNab, F., & Klingberg, T. (2008). Prefrontal cortex and basal ganglia control access to working memory. Nature neuroscience, 11(1), 103-7. doi:10.1038/nn2024
Mohr, J. P., Weiss, G. H., Caveness, W. F., Dillon, J. D., Kistler, J. P., Meirowsky, A. M., & Rish, B. L. (1980). Language and motor disorders after penetrating head injury in Viet Nam. Neurology, 30(12), 1273-9. Retrieved from http://www.ncbi.nlm.nih.gov/pubmed/7192808
Morris, R. W., Weickert, C. S., & Loughland, C. M. (2009). Emotional face processing in schizophrenia. Current opinion in psychiatry, 22(2), 140-6. doi:10.1097/YCO.0b013e328324f895
Morrison, A. L., King, T. M., Korell, M. A., Smialek, J. E., & Troncoso, J. C. (1998). Acceleration-deceleration injuries to the brain in blunt force trauma. The American journal of forensic medicine and pathology, 19(2), 109-12. Retrieved from http://www.ncbi.nlm.nih.gov/pubmed/9662103
Mula, M., & Trimble, M. R. (2009). Antiepileptic drug-induced cognitive adverse effects: potential mechanisms and contributing factors. CNS drugs, 23(2), 121-37. Retrieved from http://www.ncbi.nlm.nih.gov/pubmed/19173372
Murphy, K. R., & Barkley, R. A. (1996). Prevalence of DSM-IV symptoms of ADHD in adult licensed drivers: Implications for clinical diagnosis. Journal of Attention Disorders, 1(3), 147-161. Sage Publications. doi:10.1177/108705479600100303
Müller, R.-A. (2007). The study of autism as a distributed disorder. Mental retardation and developmental disabilities research reviews, 13(1), 85-95. doi:10.1002/mrdd.20141
NINDA Traumatic Brain Injury Information Page. (2008). National Institute of Neurological Disorders and Stroke. Retrieved from http://www.ninds.nih.gov/disorders/tbi/tbi.htm
Nahum, A. M., & Melvin, J. (2001). Accidental Injury: Biomechanics and Prevention (Google eBook) (Vol. 2001, p. 637). Springer.
Neurotrauma: New Insights Into Pathology and Treatment (Google eBook). (2007). (p. 443). Elsevier.
Noggle, C. A. (2011). The Encyclopedia of Neuropsychological Disorders (p. 804). Springer Publishing Company. Retrieved from http://books.google.com/books?id=Q30_QeaGvLYC&pgis=1
Oblak A, Gibbs T. T, & Blatt G. J. (2009). Decreased GABAA receptors and benzodiazepine binding sites in the anterior cingulate cortex in autism. Autism Research, 2(4), 205-219. doi:10.1002/aur.88. Decreased
Ommaya, A. K., & Gennarelli, T. A. (1974). Cerebral concussion and traumatic unconsciousness. Correlation of experimental and clinical observations of blunt head injuries. Brain: a journal of neurology, 97(4), 633-54. Retrieved from http://www.ncbi.nlm.nih.gov/pubmed/4215541
Picchioni, M. M., & Murray, R. M. (2007). Schizophrenia. BMJ (Clinical research ed.), 335(7610), 91-5. doi:10.1136/bmj.39227.616447.BE
Poirier, M. P. (2003). Concussions: assessment, management, and recommendations for return to activity. Clinical Pediatric Emergency Medicine, 4(3), 179-185. doi:10.1016/S1522-8401(03)00061-2
Ponsford, J., Draper, K., & Schönberger, M. (2008). Functional outcome 10 years after traumatic brain injury: its relationship with demographic, injury severity, and cognitive and emotional status. Journal of the International Neuropsychological Society: JINS, 14(2), 233-42. doi:10.1017/S1355617708080272
Porges, S. W. (1998). Love: an emergent property of the mammalian autonomic nervous system. Psychoneuroendocrinology, 23(8), 837-61. Retrieved from http://www.ncbi.nlm.nih.gov/pubmed/9924740
Porges, S. W. (2001). The polyvagal theory: phylogenetic substrates of a social nervous system. International journal of psychophysiology: official journal of the International Organization of Psychophysiology, 42(2), 123-46. Retrieved from http://www.ncbi.nlm.nih.gov/pubmed/11587772
Porges, S. W. (2003a). The Polyvagal Theory: phylogenetic contributions to social behavior. Physiology & behavior, 79(3), 503-13. Retrieved from http://www.ncbi.nlm.nih.gov/pubmed/12954445
Porges, S. W. (2003b). Social engagement and attachment: a phylogenetic perspective. Annals of the New York Academy of Sciences, 1008, 31-47. Retrieved from http://www.ncbi.nlm.nih.gov/pubmed/14998870
Porges, S. W. (2007). The polyvagal perspective. Biological psychology, 74(2), 116-43. doi:10.1016/j.biopsycho.2006.06.009
Porges, S. W. (2009). The polyvagal theory: new insights into adaptive reactions of the autonomic nervous system. Cleveland Clinic journal of medicine, 76 Suppl 2(April), S86-90. doi:10.3949/ccjm.76.s2.17
Povlishock, J. T. (2008). The classification of traumatic brain injury (TBI) for targeted therapies. Journal of neurotrauma, 25(7), 717-8. doi:10.1089/neu.2008.9964
Professor, A. C. P. S. M. M. Frcp. F. & E. (2002). Symptoms in the Mind: An Introduction to Descriptive Psychopathology (p. 430). Saunders Ltd. Retrieved from http://www.amazon.co.uk/Symptoms-Mind-Introduction-Descriptive-Psychopathology/dp/0702026271
Reichenberg, A. (2005, January). Cognitive impairment as a risk factor for psychosis. Dialogues in clinical neuroscience. Retrieved from http://www.ncbi.nlm.nih.gov/pubmed/21946140
Ridder, W. H., Borsting, E., Cooper, M., McNeel, B., & Huang, E. (1997). Not all dyslexics are created equal. Optometry and vision science: official publication of the American Academy of Optometry, 74(2), 99-104. Retrieved from http://www.ncbi.nlm.nih.gov/pubmed/9097326
Romani, A., Conte, S., Callieco, R., Bergamaschi, R., Versino, M., Lanzi, G., Zambrino, C. A., et al. (2001). Visual evoked potential abnormalities in dyslexic children. Functional neurology, 16(3), 219-29. Retrieved from http://www.ncbi.nlm.nih.gov/pubmed/11769867
Rubenstein, J. L. R., & Merzenich, M. M. (2003). Model of autism: increased ratio of excitation/inhibition in key neural systems. Genes, brain, and behavior, 2(5), 255-67. Retrieved from http://www.ncbi.nlm.nih.gov/pubmed/14606691
Russell, T. A., Green, M. J., Simpson, I., & Coltheart, M. (2008). Remediation of facial emotion perception in schizophrenia: concomitant changes in visual attention. Schizophrenia research, 103(1-3), 248-56. doi:10.1016/j.schres.2008.04.033
Silver, H., Goodman, C., Knoll, G., & Isakov, V. (2004). Brief emotion training improves recognition of facial emotions in chronic schizophrenia. A pilot study. Psychiatry research, 128(2), 147-54. doi:10.1016/j.psychres.2004.06.002
Silverstein, S. M., Schenkel, L. S., Valone, C., & Nuernberger, S. W. (1998). Cognitive deficits and psychiatric rehabilitation outcomes in schizophrenia. The Psychiatric quarterly, 69(3), 169-91. Retrieved from http://www.ncbi.nlm.nih.gov/pubmed/9682284
Smith, B., Fowler, D. G., Freeman, D., Bebbington, P., Bashforth, H., Garety, P., Dunn, G., et al. (2006). Emotion and psychosis: links between depression, self-esteem, negative schematic beliefs and delusions and hallucinations. Schizophrenia research, 86(1-3), 181-8. doi:10.1016/j.schres.2006.06.018
Smith, D. H., Meaney, D. F., & Shull, W. H. (1989). Diffuse axonal injury in head trauma. The Journal of head trauma rehabilitation, 18(4), 307-16. Retrieved from http://www.ncbi.nlm.nih.gov/pubmed/16222127
Smith, T., Weston, C., & Lieberman, J. (2010). Schizophrenia (maintenance treatment). American family physician, 82(4), 338-9. Retrieved from http://www.ncbi.nlm.nih.gov/pubmed/20704164
Solomon, M., Ozonoff, S. J., Ursu, S., Ravizza, S., Cummings, N., Ly, S., & Carter, C. S. (2009). The neural substrates of cognitive control deficits in autism spectrum disorders. Neuropsychologia, 47(12), 2515-26. doi:10.1016/j.neuropsychologia.2009.04.019
Tager-Flusberg, H. (2010). The origins of social impairments in autism spectrum disorder: studies of infants at risk. Neural networks: the official journal of the International Neural Network Society, 23(8-9), 1072-6. doi:10.1016/j.neunet.2010.07.008
Talcott J B, Hansen P C, Willis-Owen C, McKinnell I W, Richardson A J, S. J. (1998). Visual magnocellular impairment in adult developmental dyslexics. Neuro-ophthalmology, 20, 187-201.
Tallal, P., Merzenich, M., Miller, S., & Jenkins, W. (1998). Language learning impairment: integrating research and remediation. Scandinavian journal of psychology, 39(3), 197-9. Retrieved from http://www.ncbi.nlm.nih.gov/pubmed/9800537
Taylor, J. J. (1958). Selected Writings of John Hughlings Jackson. (J. J. Taylor, Ed.) (pp. 45-118). London: Staples Press.
Textbook of Traumatic Brain Injury. (2004). (p. 792). American Psychiatric Publishing, Inc. Retrieved from http://www.amazon.com/Textbook-Traumatic-Injury-Jonathan-Silver/dp/1585621056
The Boundaries of Consciousness: Neurobiology And Neuropathology (Google eBook). (2006). (p. 585). Elsevier. Retrieved from http://books.google.com/books?id=YHGacGKyVbYC&pgis=1
The Practice of Forensic Neuropsychology: Meeting Challenges in the Courtroom (Critical Issues in Neuropsychology). (1996). (p. 240). Springer.
Tucker, P., Zaninelli, R., Yehuda, R., Ruggiero, L., Dillingham, K., & Pitts, C. D. (2001). Paroxetine in the treatment of chronic posttraumatic stress disorder: results of a placebo-controlled, flexible-dosage trial. The Journal of clinical psychiatry, 62(11), 860-8. Retrieved from http://www.ncbi.nlm.nih.gov/pubmed/11775045
Tunnicliff, G. (1996). Basis of the antiseizure action of phenyloin. General pharmacology, 27(7), 1091-7. Retrieved from http://www.ncbi.nlm.nih.gov/pubmed/8981053
Tuominen, H. J., Tiihonen, J., & Wahlbeck, K. (2005). Glutamatergic drugs for schizophrenia: a systematic review and meta-analysis. Schizophrenia research, 72(2-3), 225-34. doi:10.1016/j.schres.2004.05.005
Vicini, S., Alho, H., Costa, E., Mienville, J. M., Santi, M. R., & Vaccarino, F. M. (1986). Modulation of gamma-aminobutyric acid-mediated inhibitory synaptic currents in dissociated cortical cell cultures. Proceedings of the National Academy of Sciences of the United States of America, 83(23), 9269-73. Retrieved from http://www.pubmedcentral.nih.gov/articlerender.fcgi?artid=387117&tool=pmcentrez&rende rtype=abstract
Weiss, M., Safren, S. a, Solanto, M. V., Hechtman, L., Rostain, A. L., Ramsay, J. R., & Murray, C. (2008). Research forum on psychological treatment of adults with ADHD. Journal of attention disorders, 11(6), 642-51. doi:10.1177/1087054708315063
Whyte, J. (2010). Pharmacological treatment of cognitive and behavioural sequele of traumatic brain injury: practicing in the absence of strong evidence. EUR J PHYS REHABIL MED, 46(4), 557-562.
Willcutt, E. G., & Pennington, B. F. (2000). Comorbidity of reading disability and attention-deficit/hyperactivity disorder: differences by gender and subtype. Journal of Learning Disabilities, 33(2), 179-191. doi:10.1177/002221940003300206
Williams, C., & Wood, R. L. (2010). Alexithymia and emotional empathy following traumatic brain injury. Journal of clinical and experimental neuropsychology, 32(3), 259-67. doi:10.1080/13803390902976940
Wingo, A. P., Harvey, P. D., & Baldessarini, R. J. (2009). Neurocognitive impairment in bipolar disorder patients: functional implications. Bipolar disorders, 11(2), 113-25. doi:10.111141399-5618.2009.00665.x
Zhang, L. I., Bao, S., & Merzenich, M. M. (2001). Persistent and specific influences of early acoustic environments on primary auditory cortex. Nature neuroscience, 4(11), 1123-30. doi:10.1038/nn745
van Os, J., & Kapur, S. (2009). Schizophrenia. Lancet, 374(9690), 635-45. doi:10.1016/50140-6736(09)60995-8
Adler, L. A., & Cohen, J. (2004). Diagnosis and evaluation of adults with attention deficit hyperactivity disorder. Psychiatry Clinic, 27, 187-214.
Arnott, W., Sali, L., & Copland, D. (2011). Impaired reading comprehension in schizophrenia: evidence for underlying phonological processing deficits. Psychiatry research, 187(1-2), 6-10. Elsevier Ireland Ltd. doi:10.1016/j.psychres.2010.11.025
Association, A. P. (2000). Diagnostic and Statistical Manual of Mental Disorders, Fourth Edition: DSM-IV-TR® (p. 943). American Psychiatric Pub. Retrieved from http://books.google.com/books?id=w_HajjMnjxwC&pgis=1
Bales, J., Wagner, A., & Kline, A. (2009). Persistent cognitive dysfunction after traumatic brain injury: A dopamine hypothesis. Neurosci Biobehav Rev., 33(7), 981-1003. doi:10.1016/j.neubiorev.2009.03.011. Persistent
Basso, A., & Scarpa, M. T. (1990). Traumatic aphasia in children and adults: a comparison of clinical features and evolution. Cortex; a journal devoted to the study of the nervous system and behavior, 26(4), 501-14. Retrieved from http://www.ncbi.nlm.nih.gov/pubmed/1706973
Bayly, P. V., Cohen, T. S., Leister, E. P., Ajo, D., Leuthardt, E. C., & Genin, G. M. (2005). Deformation of the human brain induced by mild acceleration. Journal of neurotrauma, 22(8), 845-56. doi:10.1089/neu.2005.22.845
Becker, T., & Kilian, R. (2006). Psychiatric services for people with severe mental illness across western Europe: what can be generalized from current knowledge about differences in provision, costs and outcomes of mental health care? Acta psychiatrica Scandinavica. Supplementum, (429), 9-16. doi:10.1111/j.1600-0447.2005.00711.x
Bell, V., Halligan, P. W., & Ellis, H. D. (2006). Explaining delusions: a cognitive perspective. Trends in cognitive sciences, 10(5), 219-26. doi:10.1016/j.tics.2006.03.004
Bentall, R. P., Fernyhough, C., Morrison, A. P., Lewis, S., & Corcoran, R. (2007). Prospects for a cognitive-developmental account of psychotic experiences. The British journal of clinical psychology/the British Psychological Society, 46(Pt 2), 155-73. doi:10.1348/014466506X123011
Betancur, C. (2011). Etiological heterogeneity in autism spectrum disorders: more than 100 genetic and genomic disorders and still counting. Brain research, 1380, 42-77. doi:10.1016/j.brainres.2010.11.078
Biederman, J., Faraone, S. V., Monuteaux, M. C., & Biederman, J., Faraone, S. V., & Monuteaux, M. (2002). Differential effect of environmental adversity by gender: Rutter's index of adversity in a sample of boys and girls with and without ADHD. American Journal of Psychiatry, 159(9), 1556-1563. American Psychiatric Assn. doi:10.1176/appi.ajp.159.9.1556
Blissitt, P. A. (2006a). Care of the critically ill patient with penetrating head injury. Critical care nursing clinics of North America, 18(3), 321-32. doi:10.1016/j.ccell.2006.05.006
Blissitt, P. A. (2006b). Care of the critically ill patient with penetrating head injury. Critical care nursing clinics of North America, 18(3), 321-32. doi:10.1016/j.ccell.2006.05.006
Borg, E., & Counter, S. A. (1989). The middle-ear muscles. Scientific American, 261(2), 74-80. Retrieved from http://www.ncbi.nlm.nih.gov/pubmed/2667133
Brady, K., Pearlstein, T., Asnis, G. M., Baker, D., Rothbaum, B., Sikes, C. R., & Farfel, G. M. (2000). Efficacy and safety of sertraline treatment of posttraumatic stress disorder: a randomized controlled trial. JAMA: the journal of the American Medical Association, 283(14), 1837-44. Retrieved from http://www.ncbi.nlm.nih.gov/pubmed/10770145
Bramlett, H. M., & Dietrich, W. D. (2007). Progressive damage after brain and spinal cord injury: pathomechanisms and treatment strategies. Progress in brain research, 161, 125-41. doi:10.1016/S0079-6123(06)61009-1
Brekke, J., Kay, D. D., Lee, K. S., & Green, M. F. (2005). Biosocial pathways to functional outcome in schizophrenia. Schizophrenia Research, 80(2-3), 213-225. Retrieved from http://www.ncbi.nlm.nih.gov/pubmed/16137859
Bremner, J. D., Mletzko, T., Welter, S., Quinn, S., Williams, C., Brummer, M., Siddiq, S., et al. (2005). Effects of phenyloin on memory, cognition and brain structure in post-traumatic stress disorder: a pilot study. Journal of psychopharmacology (Oxford, England), 19(2), 159-65. doi:10.1177/0269881105048996
Buckley, P. F., Miller, B. J., Lehrer, D. S., & Castle, D. J. (2009). Psychiatric comorbidities and schizophrenia. Schizophrenia bulletin, 35(2), 383-402. doi:10.1093/schbul/sbn135
Busch, R. M., McBride, A., Curtiss, G., & Vanderploeg, R. D. (2005). The components of executive functioning in traumatic brain injury. Journal of clinical and experimental neuropsychology, 27(8), 1022-32. doi:10.1080/13803390490919263
Cienfuegos, A., March, L., Shelley, A. M., & Javitt, D. C. (1999). Impaired categorical perception of synthetic speech sounds in schizophrenia. Biological psychiatry, 45(1), 82-8. Retrieved from http://www.ncbi.nlm.nih.gov/pubmed/9894579
Cohen, A. S., & Docherty, N. M. (2004). Affective reactivity of speech and emotional experience in patients with schizophrenia. Schizophrenia research, 69(1), 7-14. doi:10.1016/50920-9964(03)00069-0
Committee on Child Abuse and Neglect. (2001). Shaken Baby Syndrome: Rotational Cranial Injuries Technical Report. PEDIATRICS, 108(1), 206-210. doi:10.1542/peds.108.1.206
Costa, E., & Guidotti, A. (1996). Benzodiazepines on trial: a research strategy for their rehabilitation. Trends in pharmacological sciences, 17(5), 192-200. Retrieved from http://www.ncbi.nlm.nih.gov/pubmed/8669126
Coyle, J. T., Tsai, G., & Goff, D. (2003). Converging evidence of NMDA receptor hypofunction in the pathophysiology of schizophrenia. Annals of the New York Academy of Sciences, 1003, 318-27. Retrieved from http://www.ncbi.nlm.nih.gov/pubmed/14684455
Cummings, J. L. (1993). Frontal-subcortical circuits and human behavior. Archives of neurology, 50(8), 873-80. Retrieved from http://www.ncbi.nlm.nih.gov/pubmed/8352676
Davids, E., K is, B., Specka, M., & Gastpar, M. (2006). A pilot clinical trial of oxcarbazepine in adults with attention-deficit hyperactivity disorder. Progress in neuropsychopharmacology biological psychiatry (Vol. 30, pp. 1033-1038).
Demb, J. B., Boynton, G. M., Best, M., & Heeger, D. J. (1998). Psychophysical evidence for a magnocellular pathway deficit in dyslexia. Vision research, 38(11), 1555-9. Retrieved from http://www.ncbi.nlm.nih.gov/pubmed/9747491
Erdmann, J. (2011). Broad collaborations bring new energy to autism therapeutics. Chemistry & biology, 18(2), 142-3. doi:10.1016/j.chembiol.2011.02.006
Faden, A., Demediuk, P., Panter, S., & Vink, R. (1989). The role of excitatory amino acids and NMDA receptors in traumatic brain injury. Science, 244(4906), 798-800. doi:10.1126/science.2567056
Faraone, S. V., Biederman, J., Spencer, T., Mick, E., Murray, K., Petty, C., Adamson, J. J., et al. (2006). Diagnosing adult attention deficit hyperactivity disorder: are late onset and subthreshold diagnoses valid? The American journal of psychiatry, 163(10), 1720-9; quiz 1859. doi:10.1176/appl.ajp.163.10.1720
Forde, I., Holloway, J., Healy, O., & Brosnan, J. (2011). A dyadic analysis of the effects of setting and communication partner on elicited and spontaneous communication of children with Autism Spectrum Disorder and typically developing children. Research in Autism Spectrum Disorders, 5(4), 1471-1478.
Freeman, D., Garety, P. A., Kuipers, E., Fowler, D., Bebbington, P. E., & Dunn, G. (2007). Acting on persecutory delusions: the importance of safety seeking. Behaviour research and therapy, 45(1), 89-99. doi:10.1016/j.brat.2006.01.014
Gomot, M., & Wicker, B. (2012). A challenging, unpredictable world for people with Autism Spectrum Disorder. International journal of psychophysiology: official journal of the International Organization of Psychophysiology, 83(2), 240-7. doi:10.1016/j.ijpsycho.2011.09.017
Granacher, R. P. (2007). Traumatic Brain Injury: Methods for Clinical and Forensic Neuropsychiatric Assessment (Google eBook) (p. 560). CRC Press.
Green, M F, Kern, R. S., Braff, D. L., & Mintz, J. (2000). Neurocognitive deficits and functional outcome in schizophrenia: are we measuring the “right stuff”? Schizophrenia bulletin, 26(1), 119-36. Retrieved from http://www.ncbi.nlm.nih.gov/pubmed/10755673
Green, Michael F. (2006). Cognitive impairment and functional outcome in schizophrenia and bipolar disorder. The Journal of clinical psychiatry, 67(10), e12. Retrieved from http://www.ncbi.nlm.nih.gov/pubmed/17107235
Guastella, A. J., Mitchell, P. B., & Dadds, M. R. (2008). Oxytocin increases gaze to the eye region of human faces. Biological psychiatry, 63(1), 3-5. doi:10.1016/j.biopsych.2007.06.026
Hardman, J. M., & Manoukian, A. (2002). Pathology of head trauma. Neuroimaging clinics of North America, 12(2), 175-87, vii. Retrieved from http://www.neuroimaging.theclinics.com/article/S1052-5149(02)00009-6/fulltext
Hashimoto, T., Volk, D. W., Eggan, S. M., Mirnics, K., Pierri, J. N., Sun, Z., Sampson, A. R., et al. (2003). Gene expression deficits in a subclass of GABA neurons in the prefrontal cortex of subjects with schizophrenia. The Journal of neuroscience: the official journal of the Society for Neuroscience, 23(15), 6315-26. Retrieved from http://www.ncbi.nlm.nih.gov/pubmed/12867516
Heimberg, C., Gur, R. E., Erwin, R. J., Shtasel, D. L., & Gur, R. C. (1992). Facial emotion discrimination: III. Behavioral findings in schizophrenia. Psychiatry research, 42(3), 253-65. Retrieved from http://www.ncbi.nlm.nih.gov/pubmed/1496057
Horan, W. P., & Blanchard, J. J. (2003). Emotional responses to psychosocial stress in schizophrenia: the role of individual differences in affective traits and coping. Schizophrenia research, 60(2-3), 271-83. Retrieved from http://www.ncbi.nlm.nih.gov/pubmed/12591589
Insel, T. R. (2010). Rethinking schizophrenia. Nature, 468(7321), 187-93. Nature Publishing Group. doi:10.1038/nature09552
Jackson, J. H. (1873). ON THE ANATOMICAL & PHYSIOLOGICAL LOCALISATION OF MOVEMENTS IN THE BRAIN. The Lancet, 101(2581), 232-235. doi:10.1016/50140-6736(02)63385-9
Javitt, D C, Doneshka, P., Grochowski, S., & Ritter, W. (1995). Impaired mismatch negativity generation reflects widespread dysfunction of working memory in schizophrenia. Archives of general psychiatry, 52(7), 550-8. Retrieved from http://www.ncbi.nlm.nih.gov/pubmed/7598631
Javitt, Daniel C., Shelley, A.-M., Silipo, G., & Lieberman, J. A. (2000). Deficits in Auditory and Visual Context-Dependent Processing in Schizophrenia. ARCH GEN PSYCHIATRY, 57, 1131-1137.
Jones, H. M., & Pilowsky, L. S. (2002). Dopamine and antipsychotic drug action revisited. The British journal of psychiatry: the journal of mental science, 181, 271-5. Retrieved from http://www.ncbi.nlm.nih.gov/pubmed/12356650
Kadhim, H. J., Duchateau, J., & Sébire, G. (2008). Cytokines and brain injury: invited review. Journal of intensive care medicine, 23(4), 236-49. doi:10.1177/0885066608318458
Kessler, R C, Sonnega, A., Bromet, E., Hughes, M., & Nelson, C. B. (1995). Posttraumatic stress disorder in the National Comorbidity Survey. Archives of general psychiatry, 52(12), 1048-60. Retrieved from http://www.ncbi.nlm.nih.gov/pubmed/7492257
Kessler, Ronald C, Adler, L., Barkley, R., Biederman, J., Conners, C. K., Demler, O., Faraone, S. V., et al. (2006). The prevalence and correlates of adult ADHD in the United States: results from the National Comorbidity Survey Replication. The American journal of psychiatry, 163(4), 716-23. doi:10.1176/appl.ajp.163.4.716
Kim, E. (2002). Agitation, aggression, and disinhibition syndromes after traumatic brain injury. NeuroRehabilitation, 17(4), 297-310. Retrieved from http://www.ncbi.nlm.nih.gov/pubmed/12547978
Kneisl, C. R., & Trigoboff, E. (2008). Contemporary Psychiatric-Mental Health Nursing (2nd Edition) (p. 1024). Prentice Hall. Retrieved from http://www.amazon.com/Contemporary-Psychiatric-Mental-Health-Nursing-Edition/dp/013243489X
Konradi, C., & Heckers, S. (2003). Molecular aspects of glutamate dysregulation: implications for schizophrenia and its treatment. Pharmacology & therapeutics, 97(2), 153-79. Retrieved from http://www.ncbi.nlm.nih.gov/pubmed/12559388
Kraus, M. F., Susmaras, T., Caughlin, B. P., Walker, C. J., Sweeney, J. A., & Little, D. M. (2007). White matter integrity and cognition in chronic traumatic brain injury: a diffusion tensor imaging study. Brain: a journal of neurology, 130(Pt 10), 2508-19. doi:10.1093/brain/awm216
Kuipers, E., Garety, P., Fowler, D., Freeman, D., Dunn, G., & Bebbington, P. (2006). Cognitive, emotional, and social processes in psychosis: refining cognitive behavioral therapy for persistent positive symptoms. Schizophrenia bulletin, 32 Suppl 1, S24-31. doi:10.1093/schbul/sbl014
Kumar, R., Husain, M., Gupta, R. K., Hasan, K. M., Haris, M., Agarwal, A. K., Pandey, C. M., et al. (2009). Serial changes in the white matter diffusion tensor imaging metrics in moderate traumatic brain injury and correlation with neuro-cognitive function. Journal of neurotrauma, 26(4), 481-95. doi:10.1089/neu.2008.0461
Kurtz, M. M. (2005). Neurocognitive impairment across the lifespan in schizophrenia: an update. Schizophrenia research, 74(1), 15-26. doi:10.1016/j.schres.2004.07.005
Kéri, S., Kiss, I., & Kelemen, O. (2009). Sharing secrets: oxytocin and trust in schizophrenia. Social neuroscience, 4(4), 287-93. doi:10.1080/17470910802319710
Lahti, A. C., Weiler, M. A., Tamara Michaelidis, B. A., Parwani, A., & Tamminga, C. A. (2001). Effects of ketamine in normal and schizophrenic volunteers. Neuropsychopharmacology: official publication of the American College of Neuropsychopharmacology, 25(4), 455-67. doi:10.1016/S0893-133X(01)00243-3
Lai, J., Porreca, F., Hunter, J. C., & Gold, M. S. (2004). Voltage-gated sodium channels and hyperalgesia. Annual review of pharmacology and toxicology, 44, 371-97. doi:10.1146/annurev.pharmtox.44.101802.121627
Laruelle, M., Abi-Dargham, A., van Dyck, C. H., Gil, R., D'Souza, C. D., Erdos, J., McCance, E., et al. (1996). Single photon emission computerized tomography imaging of amphetamine-induced dopamine release in drug-free schizophrenic subjects. Proceedings of the National Academy of Sciences of the United States of America, 93(17), 9235-40. Retrieved from http://www.pubmedcentral.nih.gov/articlerender.fcgi?artid=38625&tool=pmcentrez&render type=abstract
Latalova, K., Prasko, J., Diveky, T., & Velartova, H. (2011). Cognitive impairment in bipolar disorder. Biomedical Papers, 155(1), 19-26. doi:10.5507/bp.155.2011.003
Lewis, D. a, Volk, D. W., & Hashimoto, T. (2004). Selective alterations in prefrontal cortical GABA neurotransmission in schizophrenia: a novel target for the treatment of working memory dysfunction. Psychopharmacology, 174(1), 143-50. doi:10.1007/s00213-003-1673-x
Lux, W. E. (2007). A neuropsychiatric perspective on traumatic brain injury. The Journal of Rehabilitation Research and Development, 44(7), 951-962. doi:10.1682/JRRD.2007.01.0009
Maas, A. I. R., Stocchetti, N., & Bullock, R. (2008). Moderate and severe traumatic brain injury in adults. Lancet neurology, 7(8), 728-41. doi:10.1016/S1474-4422(08)70164-9
Manuscript, A., Revheim, N., Butler, P. D., Schechter, I., Jalbrzikowski, M., Silipo, G., & Javitt, D. C. (2006). Reading impairment and visual processing deficits in schizophrenia. Schizophrenia research, 87(1-3), 238-45. doi:10.1016/j.schres.2006.06.022
Matson, J. L., Kozlowski, A. M., Worley, J. A., Shoemaker, M. E., Sipes, M., & Horovitz, M. (2011). What is the evidence for environmental causes of challenging behaviors in persons with intellectual disabilities and autism spectrum disorders? Research in developmental disabilities, 32(2), 693-8. doi:10.1016/j.ridd.2010.11.012
Mattson, A. J., & Levin, H. S. (1990a). Frontal lobe dysfunction following closed head injury. A review of the literature. The Journal of nervous and mental disease, 178(5), 282-91. Retrieved from http://www.ncbi.nlm.nih.gov/pubmed/2187053
Mattson, A. J., & Levin, H. S. (1990b). Frontal lobe dysfunction following closed head injury. A review of the literature. The Journal of nervous and mental disease, 178(5), 282-91. Retrieved from http://www.ncbi.nlm.nih.gov/pubmed/2187053
McCrea, M. A. (2007). Mild Traumatic Brain Injury and Postconcussion Syndrome: The New Evidence Base for Diagnosis and Treatment (Oxford Workshop Series: American Academy of Clinical Neuropsychology) (p. 224). Oxford University Press, USA. Retrieved from http://www.amazon.com/Traumatic-Brain-Injury-Postconcussion-Syndrome/dp/0195328299
McDonald, S., Flanagan, S., Rollins, J., & Kinch, J. (2003). TASIT: A new clinical tool for assessing social perception after traumatic brain injury. The Journal of head trauma rehabilitation, 18(3), 219-38. Retrieved from http://www.ncbi.nlm.nih.gov/pubmed/12802165
McNab, F., & Klingberg, T. (2008). Prefrontal cortex and basal ganglia control access to working memory. Nature neuroscience, 11(1), 103-7. doi:10.1038/nn2024
Mohr, J. P., Weiss, G. H., Caveness, W. F., Dillon, J. D., Kistler, J. P., Meirowsky, A. M., & Rish, B. L. (1980). Language and motor disorders after penetrating head injury in Viet Nam. Neurology, 30(12), 1273-9. Retrieved from http://www.ncbi.nlm.nih.gov/pubmed/7192808
Morris, R. W., Weickert, C. S., & Loughland, C. M. (2009). Emotional face processing in schizophrenia. Current opinion in psychiatry, 22(2), 140-6. doi:10.1097/YCO.0b013e328324f895
Morrison, A. L., King, T. M., Korell, M. A., Smialek, J. E., & Troncoso, J. C. (1998). Acceleration-deceleration injuries to the brain in blunt force trauma. The American journal of forensic medicine and pathology, 19(2), 109-12. Retrieved from http://www.ncbi.nlm.nih.gov/pubmed/9662103
Mula, M., & Trimble, M. R. (2009). Antiepileptic drug-induced cognitive adverse effects: potential mechanisms and contributing factors. CNS drugs, 23(2), 121-37. Retrieved from http://www.ncbi.nlm.nih.gov/pubmed/19173372
Murphy, K. R., & Barkley, R. A. (1996). Prevalence of DSM-IV symptoms of ADHD in adult licensed drivers: Implications for clinical diagnosis. Journal of Attention Disorders, 1(3), 147-161. Sage Publications. doi:10.1177/108705479600100303
Müller, R.-A. (2007). The study of autism as a distributed disorder. Mental retardation and developmental disabilities research reviews, 13(1), 85-95. doi:10.1002/mrdd.20141
NINDA Traumatic Brain Injury Information Page. (2008). National Institute of Neurological Disorders and Stroke. Retrieved from http://www.ninds.nih.gov/disorders/tbi/tbi.htm
Nahum, A. M., & Melvin, J. (2001). Accidental Injury: Biomechanics and Prevention (Google eBook) (Vol. 2001, p. 637). Springer.
Neurotrauma: New Insights Into Pathology and Treatment (Google eBook). (2007). (p. 443). Elsevier.
Noggle, C. A. (2011). The Encyclopedia of Neuropsychological Disorders (p. 804). Springer Publishing Company. Retrieved from http://books.google.com/books?id=Q30_QeaGvLYC&pgis=1
Oblak A, Gibbs T. T, & Blatt G. J. (2009). Decreased GABAA receptors and benzodiazepine binding sites in the anterior cingulate cortex in autism. Autism Research, 2(4), 205-219. doi:10.1002/aur.88. Decreased
Ommaya, A. K., & Gennarelli, T. A. (1974). Cerebral concussion and traumatic unconsciousness. Correlation of experimental and clinical observations of blunt head injuries. Brain: a journal of neurology, 97(4), 633-54. Retrieved from http://www.ncbi.nlm.nih.gov/pubmed/4215541
Picchioni, M. M., & Murray, R. M. (2007). Schizophrenia. BMJ (Clinical research ed.), 335(7610), 91-5. doi:10.1136/bmj.39227.616447.BE
Poirier, M. P. (2003). Concussions: assessment, management, and recommendations for return to activity. Clinical Pediatric Emergency Medicine, 4(3), 179-185. doi:10.1016/S1522-8401(03)00061-2
Ponsford, J., Draper, K., & Schönberger, M. (2008). Functional outcome 10 years after traumatic brain injury: its relationship with demographic, injury severity, and cognitive and emotional status. Journal of the International Neuropsychological Society: JINS, 14(2), 233-42. doi:10.1017/S1355617708080272
Porges, S. W. (1998). Love: an emergent property of the mammalian autonomic nervous system. Psychoneuroendocrinology, 23(8), 837-61. Retrieved from http://www.ncbi.nlm.nih.gov/pubmed/9924740
Porges, S. W. (2001). The polyvagal theory: phylogenetic substrates of a social nervous system. International journal of psychophysiology: official journal of the International Organization of Psychophysiology, 42(2), 123-46. Retrieved from http://www.ncbi.nlm.nih.gov/pubmed/11587772
Porges, S. W. (2003a). The Polyvagal Theory: phylogenetic contributions to social behavior. Physiology & behavior, 79(3), 503-13. Retrieved from http://www.ncbi.nlm.nih.gov/pubmed/12954445
Porges, S. W. (2003b). Social engagement and attachment: a phylogenetic perspective. Annals of the New York Academy of Sciences, 1008, 31-47. Retrieved from http://www.ncbi.nlm.nih.gov/pubmed/14998870
Porges, S. W. (2007). The polyvagal perspective. Biological psychology, 74(2), 116-43. doi:10.1016/j.biopsycho.2006.06.009
Porges, S. W. (2009). The polyvagal theory: new insights into adaptive reactions of the autonomic nervous system. Cleveland Clinic journal of medicine, 76 Suppl 2(April), S86-90. doi:10.3949/ccjm.76.s2.17
Povlishock, J. T. (2008). The classification of traumatic brain injury (TBI) for targeted therapies. Journal of neurotrauma, 25(7), 717-8. doi:10.1089/neu.2008.9964
Professor, A. C. P. S. M. M. Frcp. F. & E. (2002). Symptoms in the Mind: An Introduction to Descriptive Psychopathology (p. 430). Saunders Ltd. Retrieved from http://www.amazon.co.uk/Symptoms-Mind-Introduction-Descriptive-Psychopathology/dp/0702026271
Reichenberg, A. (2005, January). Cognitive impairment as a risk factor for psychosis. Dialogues in clinical neuroscience. Retrieved from http://www.ncbi.nlm.nih.gov/pubmed/21946140
Ridder, W. H., Borsting, E., Cooper, M., McNeel, B., & Huang, E. (1997). Not all dyslexics are created equal. Optometry and vision science: official publication of the American Academy of Optometry, 74(2), 99-104. Retrieved from http://www.ncbi.nlm.nih.gov/pubmed/9097326
Romani, A., Conte, S., Callieco, R., Bergamaschi, R., Versino, M., Lanzi, G., Zambrino, C. A., et al. (2001). Visual evoked potential abnormalities in dyslexic children. Functional neurology, 16(3), 219-29. Retrieved from http://www.ncbi.nlm.nih.gov/pubmed/11769867
Rubenstein, J. L. R., & Merzenich, M. M. (2003). Model of autism: increased ratio of excitation/inhibition in key neural systems. Genes, brain, and behavior, 2(5), 255-67. Retrieved from http://www.ncbi.nlm.nih.gov/pubmed/14606691
Russell, T. A., Green, M. J., Simpson, I., & Coltheart, M. (2008). Remediation of facial emotion perception in schizophrenia: concomitant changes in visual attention. Schizophrenia research, 103(1-3), 248-56. doi:10.1016/j.schres.2008.04.033
Silver, H., Goodman, C., Knoll, G., & Isakov, V. (2004). Brief emotion training improves recognition of facial emotions in chronic schizophrenia. A pilot study. Psychiatry research, 128(2), 147-54. doi:10.1016/j.psychres.2004.06.002
Silverstein, S. M., Schenkel, L. S., Valone, C., & Nuernberger, S. W. (1998). Cognitive deficits and psychiatric rehabilitation outcomes in schizophrenia. The Psychiatric quarterly, 69(3), 169-91. Retrieved from http://www.ncbi.nlm.nih.gov/pubmed/9682284
Smith, B., Fowler, D. G., Freeman, D., Bebbington, P., Bashforth, H., Garety, P., Dunn, G., et al. (2006). Emotion and psychosis: links between depression, self-esteem, negative schematic beliefs and delusions and hallucinations. Schizophrenia research, 86(1-3), 181-8. doi:10.1016/j.schres.2006.06.018
Smith, D. H., Meaney, D. F., & Shull, W. H. (1989). Diffuse axonal injury in head trauma. The Journal of head trauma rehabilitation, 18(4), 307-16. Retrieved from http://www.ncbi.nlm.nih.gov/pubmed/16222127
Smith, T., Weston, C., & Lieberman, J. (2010). Schizophrenia (maintenance treatment). American family physician, 82(4), 338-9. Retrieved from http://www.ncbi.nlm.nih.gov/pubmed/20704164
Solomon, M., Ozonoff, S. J., Ursu, S., Ravizza, S., Cummings, N., Ly, S., & Carter, C. S. (2009). The neural substrates of cognitive control deficits in autism spectrum disorders. Neuropsychologia, 47(12), 2515-26. doi:10.1016/j.neuropsychologia.2009.04.019
Tager-Flusberg, H. (2010). The origins of social impairments in autism spectrum disorder: studies of infants at risk. Neural networks: the official journal of the International Neural Network Society, 23(8-9), 1072-6. doi:10.1016/j.neunet.2010.07.008
Talcott J B, Hansen P C, Willis-Owen C, McKinnell I W, Richardson A J, S. J. (1998). Visual magnocellular impairment in adult developmental dyslexics. Neuro-ophthalmology, 20, 187-201.
Tallal, P., Merzenich, M., Miller, S., & Jenkins, W. (1998). Language learning impairment: integrating research and remediation. Scandinavian journal of psychology, 39(3), 197-9. Retrieved from http://www.ncbi.nlm.nih.gov/pubmed/9800537
Taylor, J. J. (1958). Selected Writings of John Hughlings Jackson. (J. J. Taylor, Ed.) (pp. 45-118). London: Staples Press.
Textbook of Traumatic Brain Injury. (2004). (p. 792). American Psychiatric Publishing, Inc. Retrieved from http://www.amazon.com/Textbook-Traumatic-Injury-Jonathan-Silver/dp/1585621056
The Boundaries of Consciousness: Neurobiology And Neuropathology (Google eBook). (2006). (p. 585). Elsevier. Retrieved from http://books.google.com/books?id=YHGacGKyVbYC&pgis=1
The Practice of Forensic Neuropsychology: Meeting Challenges in the Courtroom (Critical Issues in Neuropsychology). (1996). (p. 240). Springer.
Tucker, P., Zaninelli, R., Yehuda, R., Ruggiero, L., Dillingham, K., & Pitts, C. D. (2001). Paroxetine in the treatment of chronic posttraumatic stress disorder: results of a placebo-controlled, flexible-dosage trial. The Journal of clinical psychiatry, 62(11), 860-8. Retrieved from http://www.ncbi.nlm.nih.gov/pubmed/11775045
Tunnicliff, G. (1996). Basis of the antiseizure action of phenyloin. General pharmacology, 27(7), 1091-7. Retrieved from http://www.ncbi.nlm.nih.gov/pubmed/8981053
Tuominen, H. J., Tiihonen, J., & Wahlbeck, K. (2005). Glutamatergic drugs for schizophrenia: a systematic review and meta-analysis. Schizophrenia research, 72(2-3), 225-34. doi:10.1016/j.schres.2004.05.005
Vicini, S., Alho, H., Costa, E., Mienville, J. M., Santi, M. R., & Vaccarino, F. M. (1986). Modulation of gamma-aminobutyric acid-mediated inhibitory synaptic currents in dissociated cortical cell cultures. Proceedings of the National Academy of Sciences of the United States of America, 83(23), 9269-73. Retrieved from http://www.pubmedcentral.nih.gov/articlerender.fcgi?artid=387117&tool=pmcentrez&rende rtype=abstract
Weiss, M., Safren, S. a, Solanto, M. V., Hechtman, L., Rostain, A. L., Ramsay, J. R., & Murray, C. (2008). Research forum on psychological treatment of adults with ADHD. Journal of attention disorders, 11(6), 642-51. doi:10.1177/1087054708315063
Whyte, J. (2010). Pharmacological treatment of cognitive and behavioural sequele of traumatic brain injury: practicing in the absence of strong evidence. EUR J PHYS REHABIL MED, 46(4), 557-562.
Willcutt, E. G., & Pennington, B. F. (2000). Comorbidity of reading disability and attention-deficit/hyperactivity disorder: differences by gender and subtype. Journal of Learning Disabilities, 33(2), 179-191. doi:10.1177/002221940003300206
Williams, C., & Wood, R. L. (2010). Alexithymia and emotional empathy following traumatic brain injury. Journal of clinical and experimental neuropsychology, 32(3), 259-67. doi:10.1080/13803390902976940
Wingo, A. P., Harvey, P. D., & Baldessarini, R. J. (2009). Neurocognitive impairment in bipolar disorder patients: functional implications. Bipolar disorders, 11(2), 113-25. doi:10.111141399-5618.2009.00665.x
Zhang, L. I., Bao, S., & Merzenich, M. M. (2001). Persistent and specific influences of early acoustic environments on primary auditory cortex. Nature neuroscience, 4(11), 1123-30. doi:10.1038/nn745
van Os, J., & Kapur, S. (2009). Schizophrenia. Lancet, 374(9690), 635-45. doi:10.1016/50140-6736(09)60995-8
Adler, L. A., & Cohen, J. (2004). Diagnosis and evaluation of adults with attention deficit hyperactivity disorder. Psychiatry Clinic, 27, 187-214.
Arnott, W., Sali, L., & Copland, D. (2011). Impaired reading comprehension in schizophrenia: evidence for underlying phonological processing deficits. Psychiatry research, 187(1-2), 6-10. Elsevier Ireland Ltd. doi:10.1016/j.psychres.2010.11.025
Association, A. P. (2000). Diagnostic and Statistical Manual of Mental Disorders, Fourth Edition: DSM-IV-TR® (p. 943). American Psychiatric Pub. Retrieved from http://books.google.com/books?id=w_HajjMnjxwC&pgis=1
Bales, J., Wagner, A., & Kline, A. (2009). Persistent cognitive dysfunction after traumatic brain injury: A dopamine hypothesis. Neurosci Biobehav Rev., 33(7), 981-1003. doi:10.1016/j.neubiorev.2009.03.011. Persistent
Basso, A., & Scarpa, M. T. (1990). Traumatic aphasia in children and adults: a comparison of clinical features and evolution. Cortex; a journal devoted to the study of the nervous system and behavior, 26(4), 501-14. Retrieved from http://www.ncbi.nlm.nih.gov/pubmed/1706973
Bayly, P. V., Cohen, T. S., Leister, E. P., Ajo, D., Leuthardt, E. C., & Genin, G. M. (2005). Deformation of the human brain induced by mild acceleration. Journal of neurotrauma, 22(8), 845-56. doi:10.1089/neu.2005.22.845
Becker, T., & Kilian, R. (2006). Psychiatric services for people with severe mental illness across western Europe: what can be generalized from current knowledge about differences in provision, costs and outcomes of mental health care? Acta psychiatrica Scandinavica. Supplementum, (429), 9-16. doi:10.1111/j.1600-0447.2005.00711.x
Bell, V., Halligan, P. W., & Ellis, H. D. (2006). Explaining delusions: a cognitive perspective. Trends in cognitive sciences, 10(5), 219-26. doi:10.1016/j.tics.2006.03.004
Bentall, R. P., Fernyhough, C., Morrison, A. P., Lewis, S., & Corcoran, R. (2007). Prospects for a cognitive-developmental account of psychotic experiences. The British journal of clinical psychology/the British Psychological Society, 46(Pt 2), 155-73. doi:10.1348/014466506X123011
Betancur, C. (2011). Etiological heterogeneity in autism spectrum disorders: more than 100 genetic and genomic disorders and still counting. Brain research, 1380, 42-77. doi:10.1016/j.brainres.2010.11.078
Biederman, J., Faraone, S. V., Monuteaux, M. C., & Biederman, J., Faraone, S. V., & Monuteaux, M. (2002). Differential effect of environmental adversity by gender: Rutter's index of adversity in a sample of boys and girls with and without ADHD. American Journal of Psychiatry, 159(9), 1556-1563. American Psychiatric Assn. doi:10.1176/appi.ajp.159.9.1556
Blissitt, P. A. (2006a). Care of the critically ill patient with penetrating head injury. Critical care nursing clinics of North America, 18(3), 321-32. doi:10.1016/j.ccell.2006.05.006
Blissitt, P. A. (2006b). Care of the critically ill patient with penetrating head injury. Critical care nursing clinics of North America, 18(3), 321-32. doi:10.1016/j.ccell.2006.05.006
Borg, E., & Counter, S. A. (1989). The middle-ear muscles. Scientific American, 261(2), 74-80. Retrieved from http://www.ncbi.nlm.nih.gov/pubmed/2667133
Brady, K., Pearlstein, T., Asnis, G. M., Baker, D., Rothbaum, B., Sikes, C. R., & Farfel, G. M. (2000). Efficacy and safety of sertraline treatment of posttraumatic stress disorder: a randomized controlled trial. JAMA: the journal of the American Medical Association, 283(14), 1837-44. Retrieved from http://www.ncbi.nlm.nih.gov/pubmed/10770145
Bramlett, H. M., & Dietrich, W. D. (2007). Progressive damage after brain and spinal cord injury: pathomechanisms and treatment strategies. Progress in brain research, 161, 125-41. doi:10.1016/S0079-6123(06)61009-1
Brekke, J., Kay, D. D., Lee, K. S., & Green, M. F. (2005). Biosocial pathways to functional outcome in schizophrenia. Schizophrenia Research, 80(2-3), 213-225. Retrieved from http://www.ncbi.nlm.nih.gov/pubmed/16137859
Bremner, J. D., Mletzko, T., Welter, S., Quinn, S., Williams, C., Brummer, M., Siddiq, S., et al. (2005). Effects of phenyloin on memory, cognition and brain structure in post-traumatic stress disorder: a pilot study. Journal of psychopharmacology (Oxford, England), 19(2), 159-65. doi: 10.1177/0269881105048996
Buckley, P. F., Miller, B. J., Lehrer, D. S., & Castle, D. J. (2009). Psychiatric comorbidities and schizophrenia. Schizophrenia bulletin, 35(2), 383-402. doi:10.1093/schbul/sbn135
Busch, R. M., McBride, A., Curtiss, G., & Vanderploeg, R. D. (2005). The components of executive functioning in traumatic brain injury. Journal of clinical and experimental neuropsychology, 27(8), 1022-32. doi:10.1080/13803390490919263
Cienfuegos, A., March, L., Shelley, A. M., & Javitt, D. C. (1999). Impaired categorical perception of synthetic speech sounds in schizophrenia. Biological psychiatry, 45(1), 82-8. Retrieved from http://www.ncbi.nlm.nih.gov/pubmed/9894579
Cohen, A. S., & Docherty, N. M. (2004). Affective reactivity of speech and emotional experience in patients with schizophrenia. Schizophrenia research, 69(1), 7-14. doi:10.1016/S0920-9964(03)00069-0
Committee on Child Abuse and Neglect. (2001). Shaken Baby Syndrome: Rotational Cranial Injuries Technical Report. PEDIATRICS, 108(1), 206-210. doi:10.1542/peds.108.1.206
Costa, E., & Guidotti, A. (1996). Benzodiazepines on trial: a research strategy for their rehabilitation. Trends in pharmacological sciences, 17(5), 192-200. Retrieved from http://www.ncbi.nlm.nih.gov/pubmed/8669126
Coyle, J. T., Tsai, G., & Goff, D. (2003). Converging evidence of NMDA receptor hypofunction in the pathophysiology of schizophrenia. Annals of the New York Academy of Sciences, 1003, 318-27. Retrieved from http://www.ncbi.nlm.nih.gov/pubmed/14684455
Cummings, J. L. (1993). Frontal-subcortical circuits and human behavior. Archives of neurology, 50(8), 873-80. Retrieved from http://www.ncbi.nlm.nih.gov/pubmed/8352676
Davids, E., K is, B., Specka, M., & Gastpar, M. (2006). A pilot clinical trial of oxcarbazepine in adults with attention-deficit hyperactivity disorder. Progress in neuropsychopharmacology biological psychiatry (Vol. 30, pp. 1033-1038).
Demb, J. B., Boynton, G. M., Best, M., & Heeger, D. J. (1998). Psychophysical evidence for a magnocellular pathway deficit in dyslexia. Vision research, 38(11), 1555-9. Retrieved from http://www.ncbi.nlm.nih.gov/pubmed/9747491
Erdmann, J. (2011). Broad collaborations bring new energy to autism therapeutics. Chemistry & biology, 18(2), 142-3. doi:10.1016/j.chembiol.2011.02.006
Faden, A., Demediuk, P., Panter, S., & Vink, R. (1989). The role of excitatory amino acids and NMDA receptors in traumatic brain injury. Science, 244(4906), 798-800. doi:10.1126/science.2567056
Faraone, S. V., Biederman, J., Spencer, T., Mick, E., Murray, K., Petty, C., Adamson, J. J., et al. (2006). Diagnosing adult attention deficit hyperactivity disorder: are late onset and subthreshold diagnoses valid? The American journal of psychiatry, 163(10), 1720-9; quiz 1859. doi:10.1176/appl.ajp.163.10.1720
Forde, I., Holloway, J., Healy, O., & Brosnan, J. (2011). A dyadic analysis of the effects of setting and communication partner on elicited and spontaneous communication of children with Autism Spectrum Disorder and typically developing children. Research in Autism Spectrum Disorders, 5(4), 1471-1478.
Freeman, D., Garety, P. A., Kuipers, E., Fowler, D., Bebbington, P. E., & Dunn, G. (2007). Acting on persecutory delusions: the importance of safety seeking. Behaviour research and therapy, 45(1), 89-99. doi:10.1016/j.brat.2006.01.014
Gomot, M., & Wicker, B. (2012). A challenging, unpredictable world for people with Autism Spectrum Disorder. International journal of psychophysiology: official journal of the International Organization of Psychophysiology, 83(2), 240-7. doi:10.1016/j.ijpsycho.2011.09.017
Granacher, R. P. (2007). Traumatic Brain Injury: Methods for Clinical and Forensic Neuropsychiatric Assessment (Google eBook) (p. 560). CRC Press.
Green, M F, Kern, R. S., Braff, D. L., & Mintz, J. (2000). Neurocognitive deficits and functional outcome in schizophrenia: are we measuring the “right stuff”? Schizophrenia bulletin, 26(1), 119-36. Retrieved from http://www.ncbi.nlm.nih.gov/pubmed/10755673
Green, Michael F. (2006). Cognitive impairment and functional outcome in schizophrenia and bipolar disorder. The Journal of clinical psychiatry, 67(10), e12. Retrieved from http://www.ncbi.nlm.nih.gov/pubmed/17107235
Guastella, A. J., Mitchell, P. B., & Dadds, M. R. (2008). Oxytocin increases gaze to the eye region of human faces. Biological psychiatry, 63(1), 3-5. doi:10.1016/j.biopsych.2007.06.026
Hardman, J. M., & Manoukian, A. (2002). Pathology of head trauma. Neuroimaging clinics of North America, 12(2), 175-87, vii. Retrieved from http://www.neuroimaging.theclinics.com/article/S1052-5149(02)00009-6/fulltext
Hashimoto, T., Volk, D. W., Eggan, S. M., Mirnics, K., Pierri, J. N., Sun, Z., Sampson, A. R., et al. (2003). Gene expression deficits in a subclass of GABA neurons in the prefrontal cortex of subjects with schizophrenia. The Journal of neuroscience: the official journal of the Society for Neuroscience, 23(15), 6315-26. Retrieved from http://www.ncbi.nlm.nih.gov/pubmed/12867516
Heimberg, C., Gur, R. E., Erwin, R. J., Shtasel, D. L., & Gur, R. C. (1992). Facial emotion discrimination: III. Behavioral findings in schizophrenia. Psychiatry research, 42(3), 253-65. Retrieved from http://www.ncbi.nlm.nih.gov/pubmed/1496057
Horan, W. P., & Blanchard, J. J. (2003). Emotional responses to psychosocial stress in schizophrenia: the role of individual differences in affective traits and coping. Schizophrenia research, 60(2-3), 271-83. Retrieved from http://www.ncbi.nlm.nih.gov/pubmed/12591589
Insel, T. R. (2010). Rethinking schizophrenia. Nature, 468(7321), 187-93. Nature Publishing Group. doi:10.1038/nature09552
Jackson, J. H. (1873). ON THE ANATOMICAL & PHYSIOLOGICAL LOCALISATION OF MOVEMENTS IN THE BRAIN. The Lancet, 101(2581), 232-235. doi:10.1016/50140-6736(02)63385-9
Javitt, D C, Doneshka, P., Grochowski, S., & Ritter, W. (1995). Impaired mismatch negativity generation reflects widespread dysfunction of working memory in schizophrenia. Archives of general psychiatry, 52(7), 550-8. Retrieved from http://www.ncbi.nlm.nih.gov/pubmed/7598631
Javitt, Daniel C., Shelley, A.-M., Silipo, G., & Lieberman, J. A. (2000). Deficits in Auditory and Visual Context-Dependent Processing in Schizophrenia. ARCH GEN PSYCHIATRY, 57, 1131-1137.
Jones, H. M., & Pilowsky, L. S. (2002). Dopamine and antipsychotic drug action revisited. The British journal of psychiatry: the journal of mental science, 181, 271-5. Retrieved from http://www.ncbi.nlm.nih.gov/pubmed/12356650
Kadhim, H. J., Duchateau, J., & Sebire, G. (2008). Cytokines and brain injury: invited review. Journal of intensive care medicine, 23(4), 236-49. doi:10.1177/0885066608318458
Kessler, R C, Sonnega, A., Bromet, E., Hughes, M., & Nelson, C. B. (1995). Posttraumatic stress disorder in the National Comorbidity Survey. Archives of general psychiatry, 52(12), 1048-60. Retrieved from http://www.ncbi.nlm.nih.gov/pubmed/7492257
Kessler, Ronald C, Adler, L., Barkley, R., Biederman, J., Conners, C. K., Demler, O., Faraone, S. V., et al. (2006). The prevalence and correlates of adult ADHD in the United States: results from the National Comorbidity Survey Replication. The American journal of psychiatry, 163(4), 716-23. doi:10.1176/appl.ajp.163.4.716
Kim, E. (2002). Agitation, aggression, and disinhibition syndromes after traumatic brain injury. NeuroRehabilitation, 17(4), 297-310. Retrieved from http://www.ncbi.nlm.nih.gov/pubmed/12547978
Kneisl, C. R., & Trigoboff, E. (2008). Contemporary Psychiatric-Mental Health Nursing (2nd Edition) (p. 1024). Prentice Hall. Retrieved from http://www.amazon.com/Contemporary-Psychiatric-Mental-Health-Nursing-Edition/dp/013243489X
Konradi, C., & Heckers, S. (2003). Molecular aspects of glutamate dysregulation: implications for schizophrenia and its treatment. Pharmacology & therapeutics, 97(2), 153-79. Retrieved from http://www.ncbi.nlm.nih.gov/pubmed/12559388
Kraus, M. F., Susmaras, T., Caughlin, B. P., Walker, C. J., Sweeney, J. A., & Little, D. M. (2007). White matter integrity and cognition in chronic traumatic brain injury: a diffusion tensor imaging study. Brain: a journal of neurology, 130(Pt 10), 2508-19. doi:10.1093/brain/awm216
Kuipers, E., Garety, P., Fowler, D., Freeman, D., Dunn, G., & Bebbington, P. (2006). Cognitive, emotional, and social processes in psychosis: refining cognitive behavioral therapy for persistent positive symptoms. Schizophrenia bulletin, 32 Suppl 1, S24-31. doi:10.1093/schbul/sbl014
Kumar, R., Husain, M., Gupta, R. K., Hasan, K. M., Haris, M., Agarwal, A. K., Pandey, C. M., et al. (2009). Serial changes in the white matter diffusion tensor imaging metrics in moderate traumatic brain injury and correlation with neuro-cognitive function. Journal of neurotrauma, 26(4), 481-95. doi:10.1089/neu.2008.0461
Kurtz, M. M. (2005). Neurocognitive impairment across the lifespan in schizophrenia: an update. Schizophrenia research, 74(1), 15-26. doi:10.1016/j.schres.2004.07.005
Kéri, S., Kiss, I., & Kelemen, O. (2009). Sharing secrets: oxytocin and trust in schizophrenia. Social neuroscience, 4(4), 287-93. doi: 10.1080/17470910802319710
Lahti, A. C., Weiler, M. A., Tamara Michaelidis, B. A., Parwani, A., & Tamminga, C. A. (2001). Effects of ketamine in normal and schizophrenic volunteers. Neuropsychopharmacology: official publication of the American College of Neuropsychopharmacology, 25(4), 455-67. doi:10.1016/50893-133X(01)00243-3
Lai, J., Porreca, F., Hunter, J. C., & Gold, M. S. (2004). Voltage-gated sodium channels and hyperalgesia. Annual review of pharmacology and toxicology, 44, 371-97. doi:10.1146/annurev.pharmtox.44.101802.121627
Laruelle, M., Abi-Dargham, A., van Dyck, C. H., Gil, R., D'Souza, C. D., Erdos, J., McCance, E., et al. (1996). Single photon emission computerized tomography imaging of amphetamine-induced dopamine release in drug-free schizophrenic subjects. Proceedings of the National Academy of Sciences of the United States of America, 93(17), 9235-40. Retrieved from http://www.pubmedcentral.nih.gov/articlerender.fcgi?artid=38625&tool=pmcentrez&render type=abstract
Latalova, K., Prasko, J., Diveky, T., & Velartova, H. (2011). Cognitive impairment in bipolar disorder. Biomedical Papers, 155(1), 19-26. doi:10.5507/bp.155.2011.003
Lewis, D. a, Volk, D. W., & Hashimoto, T. (2004). Selective alterations in prefrontal cortical GABA neurotransmission in schizophrenia: a novel target for the treatment of working memory dysfunction. Psychopharmacology, 174(1), 143-50. doi:10.1007/s00213-003-1673-x
Lux, W. E. (2007). A neuropsychiatric perspective on traumatic brain injury. The Journal of Rehabilitation Research and Development, 44(7), 951-962. doi:10.1682/JRRD.2007.01.0009
Maas, A. I. R., Stocchetti, N., & Bullock, R. (2008). Moderate and severe traumatic brain injury in adults. Lancet neurology, 7(8), 728-41. doi:10.1016/S1474-4422(08)70164-9
Manuscript, A., Revheim, N., Butler, P. D., Schechter, I., Jalbrzikowski, M., Silipo, G., & Javitt, D. C. (2006). Reading impairment and visual processing deficits in schizophrenia. Schizophrenia research, 87(1-3), 238-45. doi:10.1016/j.schres.2006.06.022
Matson, J. L., Kozlowski, A. M., Worley, J. A., Shoemaker, M. E., Sipes, M., & Horovitz, M. (2011). What is the evidence for environmental causes of challenging behaviors in persons with intellectual disabilities and autism spectrum disorders? Research in developmental disabilities, 32(2), 693-8. doi:10.1016/j.ridd.2010.11.012
Mattson, A. J., & Levin, H. S. (1990a). Frontal lobe dysfunction following closed head injury. A review of the literature. The Journal of nervous and mental disease, 178(5), 282-91. Retrieved from http://www.ncbi.nlm.nih.gov/pubmed/2187053
Mattson, A. J., & Levin, H. S. (1990b). Frontal lobe dysfunction following closed head injury. A review of the literature. The Journal of nervous and mental disease, 178(5), 282-91. Retrieved from http://www.ncbi.nlm.nih.gov/pubmed/2187053
McCrea, M. A. (2007). Mild Traumatic Brain Injury and Postconcussion Syndrome: The New Evidence Base for Diagnosis and Treatment (Oxford Workshop Series: American Academy of Clinical Neuropsychology) (p. 224). Oxford University Press, USA. Retrieved from http://www.amazon.com/Traumatic-Brain-Injury-Postconcussion-Syndrome/dp/0195328299
McDonald, S., Flanagan, S., Rollins, J., & Kinch, J. (2003). TASIT: A new clinical tool for assessing social perception after traumatic brain injury. The Journal of head trauma rehabilitation, 18(3), 219-38. Retrieved from http://www.ncbi.nlm.nih.gov/pubmed/12802165
McNab, F., & Klingberg, T. (2008). Prefrontal cortex and basal ganglia control access to working memory. Nature neuroscience, 11(1), 103-7. doi:10.1038/nn2024
Mohr, J. P., Weiss, G. H., Caveness, W. F., Dillon, J. D., Kistler, J. P., Meirowsky, A. M., & Rish, B. L. (1980). Language and motor disorders after penetrating head injury in Viet Nam. Neurology, 30(12), 1273-9. Retrieved from http://www.ncbi.nlm.nih.gov/pubmed/7192808
Morris, R. W., Weickert, C. S., & Loughland, C. M. (2009). Emotional face processing in schizophrenia. Current opinion in psychiatry, 22(2), 140-6. doi:10.1097/YCO.0b013e328324f895
Morrison, A. L., King, T. M., Korell, M. A., Smialek, J. E., & Troncoso, J. C. (1998). Acceleration-deceleration injuries to the brain in blunt force trauma. The American journal of forensic medicine and pathology, 19(2), 109-12. Retrieved from http://www.ncbi.nlm.nih.gov/pubmed/9662103
Mula, M., & Trimble, M. R. (2009). Antiepileptic drug-induced cognitive adverse effects: potential mechanisms and contributing factors. CNS drugs, 23(2), 121-37. Retrieved from http://www.ncbi.nlm.nih.gov/pubmed/19173372
Murphy, K. R., & Barkley, R. A. (1996). Prevalence of DSM-IV symptoms of ADHD in adult licensed drivers: Implications for clinical diagnosis. Journal of Attention Disorders, 1(3), 147-161. Sage Publications. doi:10.1177/108705479600100303
Müller, R.-A. (2007). The study of autism as a distributed disorder. Mental retardation and developmental disabilities research reviews, 13(1), 85-95. doi:10.1002/mrdd.20141
NINDA Traumatic Brain Injury Information Page. (2008). National Institute of Neurological Disorders and Stroke. Retrieved from http://www.ninds.nih.gov/disorders/tbi/tbi.htm
Nahum, A. M., & Melvin, J. (2001). Accidental Injury: Biomechanics and Prevention (Google eBook) (Vol. 2001, p. 637). Springer.
Neurotrauma: New Insights Into Pathology and Treatment (Google eBook). (2007). (p. 443). Elsevier.
Noggle, C. A. (2011). The Encyclopedia of Neuropsychological Disorders (p. 804). Springer Publishing Company. Retrieved from http://books.google.com/books?id=Q30_QeaGvLYC&pgis=1
Oblak A, Gibbs T. T, & Blatt G. J. (2009). Decreased GABAA receptors and benzodiazepine binding sites in the anterior cingulate cortex in autism. Autism Research, 2(4), 205-219. doi:10.1002/aur.88. Decreased
Ommaya, A. K., & Gennarelli, T. A. (1974). Cerebral concussion and traumatic unconsciousness. Correlation of experimental and clinical observations of blunt head injuries. Brain: a journal of neurology, 97(4), 633-54. Retrieved from http://www.ncbi.nlm.nih.gov/pubmed/4215541
Picchioni, M. M., & Murray, R. M. (2007). Schizophrenia. BMJ (Clinical research ed.), 335(7610), 91-5. doi:10.1136/bmj.39227.616447.BE
Poirier, M. P. (2003). Concussions: assessment, management, and recommendations for return to activity. Clinical Pediatric Emergency Medicine, 4(3), 179-185. doi:10.1016/S1522-8401(03)00061-2
Ponsford, J., Draper, K., & Schönberger, M. (2008). Functional outcome 10 years after traumatic brain injury: its relationship with demographic, injury severity, and cognitive and emotional status. Journal of the International Neuropsychological Society: JINS, 14(2), 233-42. doi:10.1017/S1355617708080272
Porges, S. W. (1998). Love: an emergent property of the mammalian autonomic nervous system. Psychoneuroendocrinology, 23(8), 837-61. Retrieved from http://www.ncbi.nlm.nih.gov/pubmed/9924740
Porges, S. W. (2001). The polyvagal theory: phylogenetic substrates of a social nervous system. International journal of psychophysiology: official journal of the International Organization of Psychophysiology, 42(2), 123-46. Retrieved from http://www.ncbi.nlm.nih.gov/pubmed/11587772
Porges, S. W. (2003a). The Polyvagal Theory: phylogenetic contributions to social behavior. Physiology & behavior, 79(3), 503-13. Retrieved from http://www.ncbi.nlm.nih.gov/pubmed/12954445
Porges, S. W. (2003b). Social engagement and attachment: a phylogenetic perspective. Annals of the New York Academy of Sciences, 1008, 31-47. Retrieved from http://www.ncbi.nlm.nih.gov/pubmed/14998870
Porges, S. W. (2007). The polyvagal perspective. Biological psychology, 74(2), 116-43. doi:10.1016/j.biopsycho.2006.06.009
Porges, S. W. (2009). The polyvagal theory: new insights into adaptive reactions of the autonomic nervous system. Cleveland Clinic journal of medicine, 76 Suppl 2(April), S86-90. doi:10.3949/ccjm.76.s2.17
Povlishock, J. T. (2008). The classification of traumatic brain injury (TBI) for targeted therapies. Journal of neurotrauma, 25(7), 717-8. doi:10.1089/neu.2008.9964
Professor, A. C. P. S. M. M. Frcp. F. & E. (2002). Symptoms in the Mind: An Introduction to Descriptive Psychopathology (p. 430). Saunders Ltd. Retrieved from http://www.amazon.co.uk/Symptoms-Mind-Introduction-Descriptive-Psychopathology/dp/0702026271
Reichenberg, A. (2005, January). Cognitive impairment as a risk factor for psychosis. Dialogues in clinical neuroscience. Retrieved from http://www.ncbi.nlm.nih.gov/pubmed/21946140
Ridder, W. H., Borsting, E., Cooper, M., McNeel, B., & Huang, E. (1997). Not all dyslexics are created equal. Optometry and vision science: official publication of the American Academy of Optometry, 74(2), 99-104. Retrieved from http://www.ncbi.nlm.nih.gov/pubmed/9097326
Romani, A., Conte, S., Callieco, R., Bergamaschi, R., Versino, M., Lanzi, G., Zambrino, C. A., et al. (2001). Visual evoked potential abnormalities in dyslexic children. Functional neurology, 16(3), 219-29. Retrieved from http://www.ncbi.nlm.nih.gov/pubmed/11769867
Rubenstein, J. L. R., & Merzenich, M. M. (2003). Model of autism: increased ratio of excitation/inhibition in key neural systems. Genes, brain, and behavior, 2(5), 255-67. Retrieved from http://www.ncbi.nlm.nih.gov/pubmed/14606691
Russell, T. A., Green, M. J., Simpson, I., & Coltheart, M. (2008). Remediation of facial emotion perception in schizophrenia: concomitant changes in visual attention. Schizophrenia research, 103(1-3), 248-56. doi:10.1016/j.schres.2008.04.033
Silver, H., Goodman, C., Knoll, G., & Isakov, V. (2004). Brief emotion training improves recognition of facial emotions in chronic schizophrenia. A pilot study. Psychiatry research, 128(2), 147-54. doi:10.1016/j.psychres.2004.06.002
Silverstein, S. M., Schenkel, L. S., Valone, C., & Nuernberger, S. W. (1998). Cognitive deficits and psychiatric rehabilitation outcomes in schizophrenia. The Psychiatric quarterly, 69(3), 169-91. Retrieved from http://www.ncbi.nlm.nih.gov/pubmed/9682284
Smith, B., Fowler, D. G., Freeman, D., Bebbington, P., Bashforth, H., Garety, P., Dunn, G., et al. (2006). Emotion and psychosis: links between depression, self-esteem, negative schematic beliefs and delusions and hallucinations. Schizophrenia research, 86(1-3), 181-8. doi:10.1016/j.schres.2006.06.018
Smith, D. H., Meaney, D. F., & Shull, W. H. (1989). Diffuse axonal injury in head trauma. The Journal of head trauma rehabilitation, 18(4), 307-16. Retrieved from http://www.ncbi.nlm.nih.gov/pubmed/16222127
Smith, T., Weston, C., & Lieberman, J. (2010). Schizophrenia (maintenance treatment). American family physician, 82(4), 338-9. Retrieved from http://www.ncbi.nlm.nih.gov/pubmed/20704164
Solomon, M., Ozonoff, S. J., Ursu, S., Ravizza, S., Cummings, N., Ly, S., & Carter, C. S. (2009). The neural substrates of cognitive control deficits in autism spectrum disorders. Neuropsychologia, 47(12), 2515-26. doi:10.1016/j.neuropsychologia.2009.04.019
Tager-Flusberg, H. (2010). The origins of social impairments in autism spectrum disorder: studies of infants at risk. Neural networks: the official journal of the International Neural Network Society, 23(8-9), 1072-6. doi:10.1016/j.neunet.2010.07.008
Talcott J B, Hansen P C, Willis-Owen C, McKinnell I W, Richardson A J, S. J. (1998). Visual magnocellular impairment in adult developmental dyslexics. Neuro-ophthalmology, 20, 187-201.
Tallal, P., Merzenich, M., Miller, S., & Jenkins, W. (1998). Language learning impairment: integrating research and remediation. Scandinavian journal of psychology, 39(3), 197-9. Retrieved from http://www.ncbi.nlm.nih.gov/pubmed/9800537
Taylor, J. J. (1958). Selected Writings of John Hughlings Jackson. (J. J. Taylor, Ed.) (pp. 45-118). London: Staples Press.
Textbook of Traumatic Brain Injury. (2004). (p. 792). American Psychiatric Publishing, Inc. Retrieved from http://www.amazon.com/Textbook-Traumatic-Injury-Jonathan-Silver/dp/1585621056
The Boundaries of Consciousness: Neurobiology And Neuropathology (Google eBook). (2006). (p. 585). Elsevier. Retrieved from http://books.google.com/books?id=YHGacGKyVbYC&pgis=1
The Practice of Forensic Neuropsychology: Meeting Challenges in the Courtroom (Critical Issues in Neuropsychology). (1996). (p. 240). Springer.
Tucker, P., Zaninelli, R., Yehuda, R., Ruggiero, L., Dillingham, K., & Pitts, C. D. (2001). Paroxetine in the treatment of chronic posttraumatic stress disorder: results of a placebo-controlled, flexible-dosage trial. The Journal of clinical psychiatry, 62(11), 860-8. Retrieved from http://www.ncbi.nlm.nih.gov/pubmed/11775045
Tunnicliff, G. (1996). Basis of the antiseizure action of phenyloin. General pharmacology, 27(7), 1091-7. Retrieved from http://www.ncbi.nlm.nih.gov/pubmed/8981053
Tuominen, H. J., Tiihonen, J., & Wahlbeck, K. (2005). Glutamatergic drugs for schizophrenia: a systematic review and meta-analysis. Schizophrenia research, 72(2-3), 225-34. doi:10.1016/j.schres.2004.05.005
Vicini, S., Alho, H., Costa, E., Mienville, J. M., Santi, M. R., & Vaccarino, F. M. (1986). Modulation of gamma-aminobutyric acid-mediated inhibitory synaptic currents in dissociated cortical cell cultures. Proceedings of the National Academy of Sciences of the United States of America, 83(23), 9269-73. Retrieved from http://www.pubmedcentral.nih.gov/articlerender.fcgi?artid=387117&tool=pmcentrez&rende rtype=abstract
Weiss, M., Safren, S. a, Solanto, M. V., Hechtman, L., Rostain, A. L., Ramsay, J. R., & Murray, C. (2008). Research forum on psychological treatment of adults with ADHD. Journal of attention disorders, 11(6), 642-51. doi: 10.1177/1087054708315063
Whyte, J. (2010). Pharmacological treatment of cognitive and behavioural sequele of traumatic brain injury: practicing in the absence of strong evidence. EUR J PHYS REHABIL MED, 46(4), 557-562.
Willcutt, E. G., & Pennington, B. F. (2000). Comorbidity of reading disability and attention-deficit/hyperactivity disorder: differences by gender and subtype. Journal of Learning Disabilities, 33(2), 179-191. doi:10.1177/002221940003300206
Williams, C., & Wood, R. L. (2010). Alexithymia and emotional empathy following traumatic brain injury. Journal of clinical and experimental neuropsychology, 32(3), 259-67. doi:10.1080/13803390902976940
Wingo, A. P., Harvey, P. D., & Baldessarini, R. J. (2009). Neurocognitive impairment in bipolar disorder patients: functional implications. Bipolar disorders, 11(2), 113-25. doi:10.111141399-5618.2009.00665.x
Zhang, L. I., Bao, S., & Merzenich, M. M. (2001). Persistent and specific influences of early acoustic environments on primary auditory cortex. Nature neuroscience, 4(11), 1123-30. doi:10.1038/nn745
van Os, J., & Kapur, S. (2009). Schizophrenia. Lancet, 374(9690), 635-45. doi:10.1016/50140-6736(09)60995-8
Burmeister, R., Hannay, H. J., Copeland, K., Fletcher, J. M., Boudousquie, A & Dennis, M. (2005). Attention problems and executive functions in children with spina bifida and hydrocephalus. Child Neuropsychology, 11, 265-283.
Fletcher, J. M., & Brei, T. J. (2010). Introduction: Spina Bifida-A multidisciplinary perspective. Developmental Disabilities Research Reviews, 16, 1-5.
Holmbeck, G. N., Essner, B., Kelly, L., Friedman, D., DeLucia, C., Zebracki, K & Jandasek, B. (2010). Trajectories of psychosocial adjustment in adolescents with spina bifida: a six year, four-wave longitudinal follow-up. Journal of Consulting and Clinical Psychology, 78, 511-525.
Holmbeck, G. N., Westhoven, V. C., Phillips, W. S., Bowers, R., Gruse, C., Nikolopoulos, T., Wienke Totura, C. M., & Davison, K. (2003). A multimethod, a multi-informant, and multidimensional perspective on psychosocial adjustment in preadolescents with spina bifida. Journal of Consulting and Clinical Psychology, 71, 782-796.
Oakeshott, P., Hunt, G. M., Poulton, A., & Reid, F. (2009). Expectation of life and unexpected death in open spina bifida: a 40-year complete, non-selective, longitudinal cohort study. Developmental Medicine & Child Neurology, 52, 749-753.
Rose, B. M., & Holmbeck, G, N. (2007). Attention and executive functions in adolescents with spina bifida. Journal of Pediatric Psychology, 32, 983-994.
Demyltenaere, K., Bruffaerts, R., Lee, S., Posada-Villa, J., Kovess, V., Angermeyer, M. C., Von Korff, M. (2007). Mental disorders among persons with chronic back or neck pain: Results from the world mental health surveys. Pain, 129, 332-342.
Lai, J., Porreca, F., Hunter, J, C., & Gold, M. S. (2004). Voltage gated sodium channels and hyperalgesia. Annual Review of Pharmacology and Toxicology, 44, 371-397.
Rogers, B, A. Diagnosis and management of degenerative neck pain—clinical practice. (2010). British Journal of Hospital Medicine, 71, 137-142.
Townsend, C. O., Sletten, C. D., Bruce, B. K., Rome, J. D., Luedtke, C. A., & Hodgson, J. E. (2005). Physical and emotional functioning of adult patients with chronic abdominal pain: Comparison with patients with chronic back pain. The Journal of Pain, 6, 75-83.
Von Korff, M., Crane, P., Lane, M., Miglioretti, D. L., Simon, G., Saunders, K., Stang, P., Brandenburg, & Kessler, R. (2005). Chronic spinal pain and physical-mental comorbidity in the United States: results from the national comorbidity survey replication. Pain, 113, 331-339.
Von Korff, M., & Dunn, K. M. (2008). Chronic pain reconsidered. Pain, 138, 267-276.
Young Casey, C., Greenberg, M. A., Nicassio, P.M., Harpin, R. N., & Hubbard, D. (2008). Transition from acute to chronic pain and disability: A model including cognitive, affective and trauma factors. Pain, 134, 69-79.

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

So that the invention may be readily understood and put into practical effects, reference is made to the following non-limiting examples:

### EXAMPLE 1

### Mild Cognitive Impairment (MCI) and Dementia

Patient 1A was an elderly woman with history of impairment of cognitive function. Presumptive diagnosis of possible dementia of Alzheimers type. Commenced on 25 mg of Phenyloin with some improvement but benefit was lost on continuation of this dose. Withdrawal of medication resulted in transient improvement followed by a return to pre-phenyloin functioning. Assumption made that the regular dose of 25 mg was too high. Phenyloin dose was gradually reduced until sustained improvement obtained at a daily dose of 3 mg in divided doses orally with significant improvement. This benefit has been sustained for over 24 months.

Patient 1B was an elderly retired male with a 7 year history of Alzheimer's type dementia treated with the cholinesterase inhibitor Donepezil. He experienced a significant improvement in his subjective cognitive functioning and as measured with the ADAS-cog (Alzheimer's Disease Assessment Scale-cognitive subscale). His cognitive function has gradually deteriorated since this time. On commencement of a test dose of the phenyloin 1 mg sublingual dose he demonstrated a significant improvement in his ability to read a standard text within 10 min of the dose. His reading was more fluent being better able to follow the meaning of the text. There was also improvement in his Stroop Test particularly in part three, the more cognitively demanding where his accuracy improved from 50 to 90%. The following day after this single dose he was more able to initiate activities which he had not undertaken for several months beforehand and did not need the frequent rests during the day which he had been taking.

It is postulated that the use of a very low dose anti-epileptic drug has enabled an improvement in cognitive processing and higher executive functioning. The usually well developed and sophisticated ability to maintain social relationships is one of the last systems to reach maturity in adolescence. It would seem therefore reasonable to expect this system to be the most sensitive to any cognitive decline. If treatment were available that could reverse or stabilise the decline, it would have a profound impact and benefit both for the individual's mental health and independence, as well as a delay in the need for more intensive and costly residential care. The benefits seen in this clinical situation would not be expected from the normal and excepted use of an anti-epileptic drug, which normally acts as a general cerebral depressant or mood stabiliser and at a dose that would normally be expected to result in exacerbation of any cognitive impairment, in contrast to the compositions and methods of the invention.

It has also been noted in other types of dementia for example in multiple sclerosis there is a slowing of processing even before the dementia is evident. This can be measured relatively early in the illness and the individual may be otherwise asymptomatic. It is hypothesised that as the inventor has noted improvement in the processing of individuals with MCI and dementia and with a low dose of the AED's together with or without a psychostimulant may improve the cognitive impairment associated with multiple sclerosis and may also enhance overall processing leading to improved psycho social function. We have also noted that the clinical tool used in some research studies to analyse processing speed in multiple sclerosis is the The Paced Auditory Serial Addition Test (PASAT) is a measure of cognitive function that specifically assesses auditory information processing speed and flexibility, we have used this tool experimentally in individuals with ADHD and cognitive impairment and demonstrated improvement following treatment with low-dose AED's.

### EXAMPLE 2

### Reading Disorder

Patient 2A, a middle-aged adult who commenced a trial of ultra low dose phenyloin, 1 mg daily had noted that her visual acuity had appeared to improve. She noticed greater contrast when reading. This had reduced her dependency upon spectacles which she had relied on for many years. As in previous examples these benefits were not sustained at a higher dose.

Patient 2B, had a history of learning and social difficulties. Following a 1 mg sub lingual dose of phenyloin, the patient described improved ability to read with pseudo word component of WAIT (Wechsler Individual Achievement test). Patient described an unexpected improvement in the clarity and contrast of the words and letters, further commenting on the improvement in her ability to both see and comprehend the words simultaneously. Patient also described a reduction in the instability or movement of the words on the page which had always been experienced when reading. Also further commented on the subjective improvement in vision, with an apparent improved depth perception. These beneficial effects were lost when the dose of the phenyloin was increased to 50 mg.

Patient 2C commenced on a trial of Ultra Low Dosephenyloin, 1 mg daily. Patient noted that her visual acuity had appeared to improve. Patient noticed greater contrast in the text when reading and this had reduced her dependency upon spectacles which she had relied on for many years. As in previous examples these benefits were not sustained at a higher dose. Patient 2D had a history of learning difficulties since childhood. The patient has been able to sustain employment with considerable effort. Reading had always been effortful and largely unrewarding. Patient had described difficulties tracking along the text being frequently distracted by the lines above and below becoming lost on the page as well as having little recall or comprehension of what he had read. On commencing a 0.5 mg dose of phenyloin, the patient described a significant improvement in his ability to read with less effort, able to process and understand the text and noted a significant improvement in his ability to track the written text.

The clinical improvement observed in both reading and verbal communication have both relied on enhanced effortless processing. This in turn enhances the understanding of the content of either forms of communication. In social interaction if engagement does not occur the process becomes exhausting and unrewarding, the same is true with writing or reading. The latter engagement can be conceptualised as being able to establish the picture in one's mind of the story. If this does not occur there is little benefit and thus a lack of motivation to read fiction, with the reader quickly becoming fatigued, disengaged and disinterested. These two processes can be seen in association; parallel improvements in both literacy and communication have been noted in the clinic

### EXAMPLE 3

### Attention Deficit Hyperactivity Disorder

Patient 3A: diagnosed with ADHD and generalised anxiety disorder. Treated for five years on a combination of a tricyclic antidepressant and dexamphetamine. Persistent symptoms of ADHD, in particular difficulties with organisation, selective attention, sustained attention and ability to sustain social interaction without effort. Unsuccessful attempts at withdrawal of stimulant and antidepressant with worsening of symptoms.

Trial of phenyloin 30 mg capsule temporarily associated with improvement in clarity of thoughts and eye contact during conversation both when listening and speaking. Phenyloin withdrawn, this was associated with an initial improvement then later worsening to the pre-phenyloin functioning. Medication reintroduced at a lower dose again with initial benefit that associated with rapid loss of efficacy. The improvement on ceasing the phenyloin lasted up to 7 days before the subsequent deterioration was noted. Ultra low doses of phenyloin were found to be most efficacious with a dose of 0.1 mg daily. At this dose the phenyloin was associated with the following improvements;
greater attention to details
sustained attention
less effortful listening during conversation
improved self organisation and sequencing of tasks
less distracted and forgetful
subjective improvement in clarity of thought
enhanced ability to ignore negative and intrusive thoughts
less effortful reading
improved social engagement and enjoyment of conversation

### EXAMPLE 4

### Acquired Brain Injury

### Two Cases Both in Middle-Aged Men:

1. Patient 4A had an 18 month history of closed head injury following a fall.
2. Patient 4B had a 10 year history of closed head injury following a motor vehicle accident

### Patient 4A:

The patient had suffered no loss of consciousness at the time of injury. Following the injury, he experienced significant cognitive deficits and personality change. His communication had deteriorated and he had become socially withdrawn. He was increasing reliant on his wife to provide all support.

He developed repetitive motor tics and vocalisations. The vocalizations he would experience as an increasing internal desire to do these which he could not control. Only when he was very distracted could he resist and he became worse when he was fatigued. The motor tics consisted of repetitive tapping with his thumb against his fingers. These symptoms commenced soon after his accident. He was treated with a combination of pharmacotherapy without significant benefit; he remained impaired and unable to cope in social situations. His coordination and ability to sequence thoughts was impaired which contributed to his inability undertake simple activities or repairs at home. This was in complete contrast to his premorbid functioning. He was a gifted athlete and worked as a skilled manual worker.

He was initiated on a test dose of very low phenyloin using a fragment of a commercially available tablet approximately equivalent to less than 3 mg. This was dissolved in the mouth and absorbed through the buccal mucosa. He described a dramatic and unexpected improvement in his ability to focus, his repetitive chanting and hand movements ceased. He was more able to structure his conversation which he was able to undertake with improved ability to sustain eye contact which he had not done since the accident had occurred. The dose was increased and repeated the following day on this occasion unlike on the first is there was no dramatic improvement. One the third day the same dose was repeated with a noticeable deterioration in his ability to focus and organize his thinking. Following this the phenyloin was ceased. Over the following next five days there was a gradual improvement in his functioning to a level that he experienced after the first test dose followed by a deterioration to his pre-phenyloin functioning. The hypothesis was generated that the initial dose was in the correct therapeutic range. However, following the second and third dose the efficacy of the medication was lost. It was possible following the reduction in the available phenyloin there was a transient improvement which correlated to this lower but more efficacious therapeutic range. Finally the medication levels dropped sufficiently for there to be no residual therapeutic benefit.

Since this initial trial of the sublingual dose it has been repeated on many occasions the changes have been consistent with the above hypothesis. Repeated audio and video assessments of social interaction and of reading aloud standardised texts has provided additional clinical data to confirm these findings. The assessments have also included testing using measures of rapid automised naming using the developmental Eye Movement Test and the Stroop test. These have also been consistent with the improved cognitive functioning. Both a neuropsychological and an occupational assessment have been consistent with the improvements on Ultra low phenyloin described above.

### Patient 4B:

Premorbidly a successful businessman, following the motor vehicle accident he became estranged from his family, he isolated himself and after many years obtained part-time unskilled employment. He was initially commenced on a low dose of dexamphetamine with limited improvement in his concentration and attention. On a test dose of a sublingual dose 1 mg he noted a reduction in the effort required for communication. He also spontaneously commented about the apparent enhanced clarity of vision. He described the subjective visual experience of being better able to focus on the examiners face during the assessment instead of being consistently distracted by the background of the room. There was no previous history of ADHD either in childhood or in adult life.

Commenced on a trial of phenyloin initial dose of 3 mg he described an unexpected and dramatic improvement in the ability to organise and undertake tasks. He was able the first time since the accident to focus on and follow conversation. Following the accident he was unable to read this was in contrast to his premorbid functioning where from childhood he had a read frequently and widely. Previously was unable to retain information he was reading sufficient for understanding. Following commencement of the phenyloin he began reading for the first time in 10 years both fiction and non-fiction texts.He was also able to recall details of previous conversations and information which had taken place prior to the accident. For the intervening years he had no recollection of these details and surprise both himself and his family about the depth and accuracy of his recall. This would appear to suggest an improvement or reduction in his retrograde memory loss. He was able to attend and participate in social gatherings. This was effortless and enjoyable and was able to sustain attention equivalent to that of his peers. Improvement is sufficient to begin to return to a similar level of functioning that he was able to prior to the accident.

It is clinically observed that the improvements observed in reading in the cases of acquired brain injury are possibly more significant than in other conditions where there is a developmental disorder which may have contributed to the reading difficulty. We hypothesise that in the acquired brain injury the individual had an essentially normal reading system prior to the injury. Whereas in a developmental disorder the origin of the reading difficulties may be more complex and would have been present during the acquisition of reading, therefore the deficit may have been compounded by the lack of exposure to effortless reading.

In both cases the improvements have been sustained for over 12 months. The severe fatigue which used to be associated with any cognitive demand has decreased, but still can occur after demanding and sustained concentration. On occasions symptoms of irritability and sense of being overwhelmed still occur. Although, these symptoms can be partly reversed by the administration of a further dose of ULP which can provide temporary relief.

It is clinically observed that the improvements observed in reading in the cases of acquired brain injury are possibly more significant than in other conditions where there is a developmental disorder which may have contributed to the reading difficulty. We hypothesise that in the acquired brain injury the individual had an essentially normal reading system prior to the injury. Whereas in a developmental disorder the origin of the reading difficulties may be more complex and would have been present during the acquisition of reading, therefore the deficit may have been compounded by the lack of exposure to effortless reading. However, these are early observations and will require further investigation.

### EXAMPLE 5

### Visual fatigue

### Patient 5a

Adult male with a history of ADHD stabilised on a combination of a psychostimulant and sodium valproate. Phenyloin 4 mg was added to his treatment regime with a further improvement in his overall functioning. Generally felt more mentally alert and able to focus on tasks with less effort. Patient had previously been aware of deterioration in his vision towards the end of each day and he reported that his optometrist had confirmed that the symptoms were consistent with visual fatigue and recommended the use of spectacles. He unexpectedly reported that on the commencement of the phenyloin his visual fatigue no longer occurred and he was no longer reliant on his glasses which he had been previously.

### Patient 5b

Adult diagnosed with ADHD and stable on treatment with psychostimulants. Noted a significant improvement in function when therapy augmented with a very low dose of phenyloin, 2 mg daily. Previously always relied on glasses to read because of an astigmatism. He was surprised by an apparent improvement in his visual acuity when he could unexpectedly complete a reading test without wearing his corrective glasses. He was unable to describe similar recent episodes consistent with an improvement in his visual acuity as this had not occurred previously.

### Patient 5c

Adult previously diagnosed with ADHD and no longer taking stimulant medication. Commenced on 1 mg phenyloin and noticed an immediate and unexpected improvement in visual acuity whilst reading.

### Patient 5d

Adult male, noted that his ability to undertake three-dimensional delicate work requiring both visual acuity and depth perception improved on the commencement of ultra low dose phenyloin 2 mg. Although this dose beneficial 1 mg associated with the most consistent improvement.

### Patient 5e

An adult females noted that on commencement of low dose phenyloin an improvement in her ability to accurately identify distances when playing lawn bowls.

### Patient 5f

An adult noted that on commencement of low dose phenyloin improvement in visual judgement was also noted in a young man playing soccer with an enhanced ability to judge distances and coordinate the kicking of a soccer ball. This was also associated with a reduction in the distraction he usually experienced while playing soccer. He attributed this to being overwhelmed by the other players on the field which reduced his ability to think clearly enough to play himself. Whilst training alone he was able to develop reasonable ball skills. Unfortunately these were lost when playing in a team situation. This was not dissimilar to the social anxiety he experienced, commonly describing the sense of being watched by so many people on the soccer field. On taking the Ultra low dose phenyloin he was more able to effortlessly focus on his own game and be aware of the others, both the opposition and his own team.

### EXAMPLE 6

### Autism and other Pervasive Development Disorders

Patient 6A: Adult, diagnosed in childhood with autism and ADHD. A long history of behavioural disturbance with frequent episodes of violence despite treatment with high-dose antipsychotic medication. A single test dose of the sublingual phenyloin 1 mg was administered with an immediate improvement noted in his ability to socially interact and sustain limited conversation. Following this, a regular dose of phenyloin was commenced at 4 mg sustained release daily. This improvement was consistently noted both in the home environment and at the day respite centre where he attended. The high-dose antipsychotic medication has been reduced without any associated loss of recent benefit. Following a withdrawal of the phenyloin there was a return of the behavioural disturbance which abated on recommencement

Patient 6B was an adult, diagnosed in childhood with autism and ADHD. A history of behavioural disturbance and educational impairment. Initiated on 1 mg phenyloin daily. Gradually increased to 3 mg in divided doses daily. The commencement of ULDP was associated with sustained improvement in communication and family relationships together with a reduction in behavioural disturbance. These Improvements were lost when either the phenyloin was at a higher dose or withdrawn.

Patient 6C was an adult, diagnosed in childhood with autism and ADHD with a history of behavioural disturbance and educational impairment. The patient was initiated on 1 mg phenyloin daily which was gradually increased to 3 mg in divided doses daily. The commencement of the ultra low dose phenyloin was associated with sustained improvement in communication and family relationships together with a reduction in behavioural disturbance. These improvements were lost when either a higher dose of phenyloin was administered or when medication was withdrawn.

Patient 6D: Adult, diagnosed in childhood with autism, ADHD and antisocial personality traits. Significant improvement in behaviour, verbal and non-verbal communication with the introduction of 2 mg phenyloin daily. Patient C's conversation became more appropriate and consistent together with reported improvements in significant relationships. No additional benefit on increased dose of 4 mg.

Patient 6E was an adult, diagnosed with communication disorder and major depression. A significant improvement in communication was observed on initiation of 50 mg sodium valproate. The initial benefits were lost when medication was ceased. Following reintroduction with a dose of 25 mg daily, a sustained improvement in the ability to communicate with less effort and the absence of negative internal dialogue and relentless preplanning of conversations were observed.

Patient 6F was an adult with ASD. A significant improvement in behaviour, verbal and non-verbal communication was observed with the introduction of 50 mg sodium valproate daily. The patient's conversation became more appropriate and consistent and improvements in significant relationships were reported. A reduction in the reliance on alcohol to alleviate anxiety in social situations was observed. Within three days, an increasing cognitive slowing, general malaise and tiredness were observed. Sodium valproate was withdrawn and improvement was noted. Sodium valproate dose of 25 mg daily was successfully reintroduced.

Patient 6G was as adult, diagnosed in childhood with ASD. Patient exhibited chronic impairment with motor tics, repetitive behaviour and social withdrawal. Previous improvement in communication and other symptoms of ASD on introduction of sodium valproate 50 mg daily were noted, although the patient developed side-effects including low mood and irritability. Sodium valproate was withdrawn. Reintroduction of sodium valproate 25 mg on alternate days was associated with a sustained improvement in the communication, eye contact during dialogue, reduction in motor tics and greater social engagement and independently motivated activity.

In addition to these individual examples we have observed on multiple occasions similar improvements in social cognition, behaviour and empathy on the low dose of phenyloin. This has also been associated with improvements in reading and verbal understanding. These benefits have been sustained in some situations for three years. Although difficult to be objective, there seems to be good evidence of ongoing improvement. In other words these benefits do not represent a stationary reflection of an enhanced ability to learn. Improvements in coordination, in fine and gross motor control have been reported and improvement in gait which frequently is a significant issue for individuals with autistic disorders.

A much higher dose of phenyloin has been used with good effect in patients we have treated with bipolar spectrum disorders. Although uncertain of the precise mechanism of action, it would appear that individuals with autistic spectrum disorders benefit from the Ultra Low Dose Phenyloin in contrast to those with a more affective illnesses. If the dose of phenyloin is increased above the Ultra low dose level in those with bipolar spectrum disorders, a loss of efficacy is described together with a sensitivity of his which was not seen in those with bipolar spectrum disorders. The increased sensitivity in autistic spectrum disorders to psychotropic agents has been observed previously with selective serotonin reuptake inhibitors.

It is important to consider the diagnosis of ASD is a spectrum disorder rather than a categorical condition. Therefore varying degrees of social dysfunction can cause significant psychosocial impairment. This has been considered in the diagnosis of Social Communication Disorder. The spectrum impairments are not dissimilar to that identified with ASD's but are of a lesser severity. Using this description we have noted significant improvements in many patients demonstrating these symptoms with the treatment with ultra low dose phenyloin.

A new diagnostic category Social communication disorder, is likely to be included in the Diagnostic and Statistical Manual of the American Psychiatric Association version 5 (DSM V). We have noted in many cases improvements in the symptoms which are likely to be included in this diagnosis:
A. Persistent difficulties in pragmatics or the social uses of verbal and nonverbal communication in naturalistic contexts, which affects the development of social reciprocity and social relationships that cannot be explained by low abilities in the domains of word structure and grammar or general cognitive ability.
   Many patients demonstrating difficulties in the normal use of verbal and non-verbal communication leading to clumsy and awkward interaction. These difficulties are improved by the use of low dose phenyloin and other low dose antiepileptic medications
B. Persistent difficulties in the acquisition and use of spoken language, written language, and other modalities of language (e.g., sign language) for narrative, expository and conversational discourse. Symptoms may affect comprehension, production, and awareness at a discourse level individually or in any combination that are likely to endure into adolescence and adulthood, although the symptoms, domains, and modalities involved may shift with age.
   Spoken and written language is often very effortful leading to fatigue and disengagement, these impairments are specifically improved by the use of low dose phenyloin and other low dose antiepileptic medications.
C. Rule out Autism Spectrum Disorder. Autism spectrum disorder by definition encompasses pragmatic communication problems, but also includes restricted, repetitive patterns of behavior, interests or activities as part of the autism spectrum. Therefore, Autism Spectrum Disorder needs to be ruled out for Social Communication Disorder to be diagnosed. Social Communication Disorder can occur as a primary impairment or co-exist with disorders other than Autism Spectrum Disorder (e.g., Speech Disorders Learning Disorder, Intellectual Disorders).
D. Symptoms must be present in early childhood (but may not become fully manifest until speech, language, or communication demands exceed limited capacities).
   These difficulties are long-term frequently dating back to childhood, there is on occasions evidence of any environmental compensation although this frequently leads to a rigid coping strategies which can be seen as maladaptive in other situations.
E. The low social communication abilities result in functional limitations in effective communication, social participation, academic achievement, or occupational performance, alone or in any combination.

These poor social communication difficulties are spectrum conditions but are associated often with severe and disabling impairments and frequently are associated with psychiatric morbidities. The deficits which are characterised by these symptoms are often pervasive and disabling. Improvement in these areas of poor communication often results in a significant recovery in social academic and occupational performance They may be associated with other comorbidities result in considerable disability. With successful treatment with the low dose antiepileptic medications these impairments often quickly resolve leading to improvements in overall psychosocial functioning.

### EXAMPLE 7

### Schizophrenia

Patient 7A: Adult, initially diagnosed with schizophrenia in adolescence. Long history of impairment with predominant paranoid delusions controlled with antipsychotic medication although still experiencing significant relapses despite consistent adherence to the pharmacotherapy regime. Significant social impairment, unable to sustain employment and requires support. Prominent negative symptoms and social deficits, unable to sustain eye contact when speaking. Speech slow, effortful and fatiguing. Patient commenced on 2 mg phenyloin daily; family and patient reported significant improvement in ability to communicate with less effort. Prosody and rate of speech both improved.

Patient 7B: Adult initially diagnosed with schizophrenia during adolescence, following many years of prominent positive symptoms with severe behavioural disturbances and violence requiring periods of hospitalisation. Patient has never been able to sustain employment and requires significant social support to maintain functioning. Prominent thought disorder and general social disengagement. On initiation of 1 mg of sublingual phenyloin significant improvement in speech and ability to read aloud a standard text was noted. Following this, the patient is more able to initiate activities such as spontaneously assisting repairs on motor vehicle. Able to sustain and enjoy social interaction in a sheltered workshop which had never occurred previously. Positive effects abated when phenyloin withdrawn.

### EXAMPLE 8

### Bipolar Case

Patient 8A: Adult with a diagnosis of bipolar mood disorder with limited benefit on combination of antidepressant therapy and lithium carbonate, unable to work as a consequence of illness. Trial dose of phenyloin compounded 2 mg capsule resulted in a significant improvement in function this was confirmed by friends and family. This was described as the single most beneficial treatment in many years of pharmcotherapy.

The following were noted,
enhanced organisation and planning
reduced effort required for social interaction
enhanced clarity of thought
a sense of being psychologically more normal Improvements have enabled a return to part-time work.

### EXAMPLE 9

### PTSD

Patient 9A: Two year history of PTSD & Major Depression, unable to work for nine months. Little consistent improvement despite 15 months poly-psycho-pharmacotherapy including antidepressants and antipsychotic medication. Eight months specialist psychiatric treatment including specific PTSD therapy program. Recently assessed as Global Assessment of Functioning (GAF) of 30. No history of premorbid ASD or ADHD, possible mild reading difficulties. Commenced on ultra low dose phenyloin (ULDP) 2 mg daily and within 24 hours reported the following improvements:
Clarity of thoughts
A return of his ability to sustain eye contact during conversation which had been absent since the onset of his symptoms of PTSD
Improved concentration
Able to control intrusive thoughts
Reduced cognitive fatigue
Reduced hypervigilance and startle reflex
More patient, confident and consistent in interpersonal interactions
These changes confirmed by partner

During treatment dose reduced to 1 mg and increased to 4 mg both resulting in a reduction in efficacy. Improvements have been maintained over three months now actively initiating a return to work program.

Patient 9B: 40 year history of severe PTSD associated with severe re-experiencing phenomena, absence of any family relationships and social withdrawal, anger outbursts, chronic sleep disturbance exaggerated startle reflex and hypervigilance. Comorbid conditions including chronic major depression, alcohol dependence. Attended regular psychiatric treatment for in excess of five years. Treated with a combination of regular outpatient psychotherapy and pharmacotherapy including antidepressants and antipsychotic medications. Remained significantly impaired and the symptoms in excess of those required to diagnose PTSD according to DSM IV. Weight gain associated with antipsychotic medication. Trial of phenyloin 2 mg compounded capsule twice daily, within one week the following improvements were reported:
Enhanced communication
Thoughts were better paced making speech less effortful
Distressing and intrusive thoughts less intense and easier to dismiss
Thoughts were clearer
Improved sleep with less awakenings and distressing dreams.
Speaking described as less rushed with improved capacity and reduction in the amount of stuttering.
Reduced exaggerated startle reflex and hypervigilance.

Overall described a 50% improvement on commencement of the low dose phenyloin

Patient 9C: 40 year history of PTSD associated with chronic impairment in previous alcohol dependence. Previous psychiatric admissions as a consequence of condition. Long-term antidepressant therapy together with intermittent use of antipsychotic medication. Trial of 3 mg phenyloin associated with initial improved ability to dismiss and ignore distressing, intrusive thoughts to mood stability and concentration. However, became irritable and experienced word finding difficulties. Over nine months dose gradually reduced including periods of complete withdrawal which were associated with a return to his pre-phenyloin functioning including a worsening of his symptoms of PTSD. The most stable and efficacious dose was identified as phenyloin 0.5 mg daily.

Stable improvements included;
reduction of intrusive and negative thoughts
Enhanced interpersonal communication
Reduced effort of reading
Enhanced quality of sleep

### EXAMPLE 10

### Tardive Dyskinesia

Patient 10A was an elderly man with a history of post-dramatic stress disorder treated with long-term antipsychotic medication. He had over 12 months developed abnormal movements consistent with symptoms of tardive dyskinsia. He was given a test dose of 1 mg phenyloin and these abnormal facial movements ceased. After a later withdraw! of the phenyloin he was rechallenged with the same dose of phenyloin a similar benefit was observed and maintained on a regular dose of the ULDP.

### EXAMPLE 11

### Spina Bifida

Patient 11A presented with spina bifida, ADHD, learning difficulties and social anxiety treated for many years on low dose methylphenidate. Throughout schooling the patient required educational support and demonstrated impairment in executive functioning and emotional dysregulation. These difficulties contributed to poor social skills and emotional lability especially when attempting to communicate. On commencement of ultra low sublingual dose phenyloin 1 mg there was a significant improvement in:
Social interaction as observed during the consultation and reported in other situations.
Sustain conversation with less effort and greater engagement
Ability to actively contribute noted by extended family members.
Ability to read aloud improved with enhanced prosody, pacing, speed and accuracy.
Fine motor control and handwriting both described and reported by tutors

These improvements were sustained for over 12 months and were confirmed by tutors. A loss of efficacy was observed after the dose was increased to 2 mg. The optimal response was associated with 1.5 mg phenyloin daily in two divided doses. The improvements continued to improve over 12 months on a stable dose of the phenyloin and appeared to reflect an improved retention of new learning and then build on this experience. This is possibly reflecting the normalising of the learning process by the ultra low dose phenyloin.

### EXAMPLE 12

### Chronic Pain

Patient 8A presented with a 20 year history of chronic neck pain following a traumatic injury. Multiple surgical procedures and nerve blocks had provided only temporary relief. Narcotic analgesics had not produced sustained improvement. The patient described intense pain characterised by sharp stabbing pain, becoming worse on movement together with chronic and unremitting headaches. Within 3 to 4 min of administration of a sublingual dose of 1 mg phenyloin, the patient experienced a significant reduction in the intensity of the pain from 10/10(being the most severe) to 5/10(manageable). The relief lasted for approximately 10 min, during which time the patient also unexpectedly described an enhancement in his vision, which was described as being both brighter and clearer. Subsequently, there was a return of the more severe pain but not to the pre-dose level. A further sublingual dose of phenyloin 1 mg was taken 15 mins after the first dose. A similar reduction in the severity of the pain was noted.

The improvement was characterised as being similar to the relief he had experienced when undergoing a nerve block. Following the sublingual doses of phenyloin the patient, described an absence of the more chronic pain as well as greater mobility in his movement and a reduction in the severity of the intense stabbing pain. Whilst the pain was still evident he was more able to ignore and distract himself from the noxious experience. The control of the chronic pain was maintained after the initial improvement on taking a daily dose of phenyloin 3 mg modified release capsule. A further acute improvement in the freedom of movement without pain was noted on taking an additional dose of sublingual dose of phenyloin 1 mg.

### EXAMPLE 13

### Tinnitus

Tinnitus is the perception of sound within the human ear in the absence of corresponding external sound. Tinnitus is common; about 20% of people between 55 and 65 years old report symptoms on a general health questionnaire, and 11.8% on more detailed tinnitus-specific questionnaires)

The ability to control intrusive and unpleasant stimuli should be as automatic as possible enabling active attention to be focused on new and potentially more important information. This habituation of repetitive noise from either internal, such as tinnitus or external background noise enables this process of discrimination to occur. If there are impairments in the processing of information this can lead to an inability to selectively control these stimuli. Thus the perception of tinnitus can be overwhelming and distracting. We have noted benefits in individuals with tinnitus on commencement of low dose antiepileptic medications. This is also frequently associated with improved social cognition. We hypothesise that the ability to discriminate and control the relevant stimuli is associated with enhanced neuronal function which may occur from the improved neuronal modulation.

1. An anti-epileptic agent for use in the treatment of a neurological disorder other than epilepsy characterised in that the anti-epileptic agent is the sole active agent and that the daily dose of the anti-epileptic is less than 20% of the minimum daily dose which is effective for mood stabilisation or treatment of epileptic symptoms.

2. An anti-epileptic agent according to claim 1, wherein the daily dose is less than 2.5% of the minimum daily dose of the anti-epileptic agent which is effective for mood stabilisation or treatment of epileptic symptoms.

3. A pharmaceutical composition comprising a sub-therapeutic dose of an anti-epileptic agent as the sole active agent within the composition, together with a pharmaceutically acceptable carrier, diluent and/or excipient, wherein the sub-therapeutic dose is less than 20% of the minimum daily dose of the anti-epileptic agent which is effective for mood stabilisation or treatment of epileptic symptoms.

4. A pharmaceutical composition according to claim 3, wherein the composition is for use in the treatment of a neurological disorder other than epilepsy.

5. A pharmaceutical composition according to claim 3, wherein the composition is adapted for transdermal administration.

6. A pharmaceutical composition according to claim 5, wherein the transdermal administration is via the oral mucosa and the composition is in the form of powders, a capsule, a tablet, a lozenge, a troche or a pastille.

7. A pharmaceutical composition according to claim 3, wherein the composition comprises a formulation which provides a controlled release or a sustained release of at least one active present in the composition.

8. A method of treating a neurological disorder other than epilepsy in a subject in need thereof, including the step of administering to the subject an anti-epileptic agent as the sole active agent, wherein the daily dose of the anti-epileptic agent is less than 20% of the minimum daily dose which is effective for mood stabilisation or treatment of epileptic symptoms.

9. An agent according to claim 1, wherein the antiepileptic agent is selected from brivaracetam, carbamazepine, clobazam, clonazepam, ethosuximide, felbamate, gabapentin, lacosamide, lamotrigine, levetiracetam, oxcarbazepine, phenobarbital, phenyloin, pregabalin, primidone, retigabine, rufinamide, safinamide, seletracetam, talampanel, tiagabine, topiramate, valproate, vigabatrin, zonisamide, benzodiazepines, barbiturates and sedative hypnotics.

10. A pharmaceutical composition according to claim 3, further comprising:
an active selected from a stimulant, an anti-Parkinson's agent, an analgesic and an acetylcholinesterase inhibitor.

11. A pharmaceutical composition according to claim 10, wherein:
the sub-therapeutic dose is less than 2.5% of the minimum daily dose which is effective for mood stabilisation or treatment of epileptic symptoms.

12. A pharmaceutical composition according to claim 10, wherein the composition is for use in the treatment of a neurological disorder other than epilepsy.

13. A pharmaceutical composition according to claim 10, wherein the composition is adapted for transdermal administration.

14. A pharmaceutical composition according to claim 13, wherein the composition is adapted for administration via the oral mucosa and is in the form of powders, a capsule, a tablet, a lozenge, a troche or a pastille.

15. A pharmaceutical composition according to claim 10, wherein the composition comprises a formulation which provides a controlled release or a sustained release of at least one active present in the composition.

16. A method of treating a neurological disorder other than epilepsy in a subject in need thereof, including the step of administering to the subject a combination of (a) an anti-epileptic agent, and (b) an active selected from a stimulant, an anti-Parkinson's agent, an analgesic and an acetylcholinesterase inhibitor, wherein the daily dose of the anti-epileptic agent is less than 2.5% of the minimum daily dose which is effective for mood stabilisation or the treatment of epileptic symptoms.

17. A pharmaceutical composition according to claim 10, wherein the antiepileptic agent is selected from brivaracetam, carbamazepine, clobazam, clonazepam, ethosuximide, felbamate, gabapentin, lacosamide, lamotrigine, levetiracetam, oxcarbazepine, phenobarbital, phenyloin, pregabalin, primidone, retigabine, rufinamide, safinamide, seletracetam, talampanel, tiagabine, topiramate, valproate, vigabatrin, zonisamide, benzodiazepines, barbiturates and sedative hypnotics.

18. A pharmaceutical composition according to claim 10, wherein the stimulant is selected from Adrafinil, Amantadine, Armodafinil, Carphedon, Modafinil, 4-Fluoroamphetamine, 4-Fluoromethamphetamine, 4-Methylmethcathinone, 4-MTA, α-PPP, Amphechloral, Amphetamine, Dextroamphetamine, Adderall, Amphetaminil, Benzphetamine, Bupropion, Cathinone, Chlorphentermine, Clobenzorex, Clortermine, Cypena mine, Diethyl propion, Dimethoxya mph eta mine, Dimethyla mph eta mine, Dimethylcathinone, Diphenyl prolinol, Ephedrine, Epinephrine, Ethcathinone, Ethylamphetamine, Fencamfamine, Fenethylline, Fenfluramine, Fenproporex, Feprosidnine, Furfenorex, Levomethamphetamine, Lisdexamfetamine, L-lysine-damphetamine, MDMA, Mefenorex, Methamphetamine, Methcathinone, Methoxyphedrine, Methylone, Octopamine, Parahydroxyamphetamine, PMA, PMEA, PMMA, PPAP, Phendimetrazine, Phenmetrazine, Phentermine, Phenylephrine, Phenylpropanolamine, Prolintane, Propylamphetamine, Pseudoephedrine, Selegiline, Synephrine, Tenamphetamine, Xylopropamine; piperazines, BZP, MeOPP, MBZP, mCPP, 2C-B-BZP, Tropanes, Brasofensine, CFT, Cocaethylene, Cocaine, Dimethocaine, Lometopane, PIT, PTI, RTI-121, Tesofensine, Troparil, WF-23, WF-33, Cholinergics, Arecoline, Cotinine, Convulsants, Bicuculline, Gabazine, Pentetrazol, Picrotoxin, Strychnine, Thujone; Phenylaminooxazoles, 4-Methyl-aminorex, Aminorex, Clominorex, Fenozolone, Fluminorex, Pemoline, Thozalinone, Amineptine, Bemegride, BPAP, Clenbuterol, Clofenciclan, Cyclopentamine, Cyprodenate, Desoxypipradrol, Ethylphenidate, Ethamivan, Gilutensin, GYKI-52895, Hexacyclonate, lndanorex, lndatraline, lsometheptene, Mazindol, MDPV, Mesocarb, methylphenidate, Dexmethylphenidate, Naphthylisopropylamine, Nikethamide, Nocaine, Nomifensine, Phacetoperane, Phthalimidopropiophenone, Pipradrol, Prolintane, Propylhexedrine, Pyrovalerone, Tuamine, Vanoxerine, Yohimbine, Zylofuramine, Deanol, Diethylaminoethanol, Dimefline Hydrochloride, Etilamfetamine Hydrochloride, Fencamfamin Hydrochloride, Fenetylline Hydrochloride, Fenfluramine Hydrochloride, Fenproporex Hydrochloride, Lobeline Hydrochloride, Pentetrazol, and Propylhexedrine.

19. A pharmaceutical composition according to claim 10, wherein the anti-Parkinson's agent is selected from apomorphine, benserazide, benzatropine, bromocriptine, cabergoline, carbidopa, clozapine, domperidone, entacapone, levodopa, lisuride, orphenadrine, pergolide, piribedil, pramipexole, procyclidine, quetiapine, rasagiline, rivastigmine, ropinirole, rotigotine, selegiline, tolcapone, trihexyphenidyl, a dopamine agonist, a dopamine decarboxylase inhibitor, a catechol O methyl transferase (COMT) enzyme inhibitor, a monoamine oxidase-B inhibitor and an N-methyl-D-aspartate blocker.

20. A pharmaceutical composition according to claim 10, wherein the acetylcholinesterase inhibitor is selected from tacrine, donepezil, galantamine and rivastigmine.

21. The composition according to claim 3, wherein the antiepileptic agent is selected from brivaracetam, carbamazepine, clobazam, clonazepam, ethosuximide, felbamate, gabapentin, lacosamide, lamotrigine, levetiracetam, oxcarbazepine, phenobarbital, phenyloin, pregabalin, primidone, retigabine, rufinamide, safinamide, seletracetam, talampanel, tiagabine, topiramate, valproate, vigabatrin, zonisamide, benzodiazepines, barbiturates and sedative hypnotics.

22. The composition according to claim 8, wherein the antiepileptic agent is selected from brivaracetam, carbamazepine, clobazam, clonazepam, ethosuximide, felbamate, gabapentin, lacosamide, lamotrigine, levetiracetam, oxcarbazepine, phenobarbital, phenyloin, pregabalin, primidone, retigabine, rufinamide, safinamide, seletracetam, talampanel, tiagabine, topiramate, valproate, vigabatrin, zonisamide, benzodiazepines, barbiturates and sedative hypnotics.

23. The method according to claim 16, wherein the antiepileptic agent is selected from brivaracetam, carbamazepine, clobazam, clonazepam, ethosuximide, felbamate, gabapentin, lacosamide, lamotrigine, levetiracetam, oxcarbazepine, phenobarbital, phenyloin, pregabalin, primidone, retigabine, rufinamide, safinamide, seletracetam, talampanel, tiagabine, topiramate, valproate, vigabatrin, zonisamide, benzodiazepines, barbiturates and sedative hypnotics.

24. The method according to claim 16, wherein the stimulant is selected from Adrafinil, Amantadine, Armodafinil, Carphedon, Modafinil, 4-Fluoroamphetamine, 4-Fluoromethamphetamine, 4-Methylmethcathinone, 4-MTA, α-PPP, Amphechloral, Amphetamine, Dextroamphetamine, Adderall, Amphetaminil, Benzphetamine, Bupropion, Cathinone, Chlorphentermine, Clobenzorex, Clortermine, Cypena mine, Diethyl propion, Dimethoxya mph eta mine, Dimethyla mph eta mine, Dimethylcathinone, Diphenyl prolinol, Ephedrine, Epinephrine, Ethcathinone, Ethylamphetamine, Fencamfamine, Fenethylline, Fenfluramine, Fenproporex, Feprosidnine, Furfenorex, Levomethamphetamine, Lisdexamfetamine, L-lysine-damphetamine, MDMA, Mefenorex, Methamphetamine, Methcathinone, Methoxyphedrine, Methylone, Octopamine, Parahydroxyamphetamine, PMA, PMEA, PMMA, PPAP, Phendimetrazine, Phenmetrazine, Phentermine, Phenylephrine, Phenylpropanolamine, Prolintane, Propylamphetamine, Pseudoephedrine, Selegiline, Synephrine, Tenamphetamine, Xylopropamine; piperazines, BZP, MeOPP, MBZP, mCPP, 2C-B-BZP, Tropanes, Brasofensine, CFT, Cocaethylene, Cocaine, Dimethocaine, Lometopane, PIT, PTI, RTI-121, Tesofensine, Troparil, WF-23, WF-33, Cholinergics, Arecoline, Cotinine, Convulsants, Bicuculline, Gabazine, Pentetrazol, Picrotoxin, Strychnine, Thujone; Phenylaminooxazoles, 4-Methyl-aminorex, Aminorex, Clominorex, Fenozolone, Fluminorex, Pemoline, Thozalinone, Amineptine, Bemegride, BPAP, Clenbuterol, Clofenciclan, Cyclopentamine, Cyprodenate, Desoxypipradrol, Ethylphenidate, Ethamivan, Gilutensin, GYKI-52895, Hexacyclonate, lndanorex, lndatraline, lsometheptene, Mazindol, MDPV, Mesocarb, methylphenidate, Dexmethylphenidate, Naphthylisopropylamine, Nikethamide, Nocaine, Nomifensine, Phacetoperane, Phthalimidopropiophenone, Pipradrol, Prolintane, Propylhexedrine, Pyrovalerone, Tuamine, Vanoxerine, Yohimbine, Zylofuramine, Deanol, Diethylaminoethanol, Dimefline Hydrochloride, Etilamfetamine Hydrochloride, Fencamfamin Hydrochloride, Fenetylline Hydrochloride, Fenfluramine Hydrochloride, Fenproporex Hydrochloride, Lobeline Hydrochloride, Pentetrazol, and Propylhexedrine.

25. The method according to claim 16, wherein the anti-Parkinson's agent is selected from apomorphine, benserazide, benzatropine, bromocriptine, cabergoline, carbidopa, clozapine, domperidone, entacapone, levodopa, lisuride, orphenadrine, pergolide, piribedil, pramipexole, procyclidine, quetiapine, rasagiline, rivastigmine, ropinirole, rotigotine, selegiline, tolcapone, trihexyphenidyl, a dopamine agonist, a dopamine decarboxylase inhibitor, a catechol O methyl transferase (COMT) enzyme inhibitor, a monoamine oxidase-B inhibitor and an N-methyl-D-aspartate blocker.

26. The method according to claim 16, wherein the acetylcholinesterase inhibitor is selected from tacrine, donepezil, galantamine and rivastigmine.
